(19) United States
(12) Patent Application Publication
(10) Pub. No.: US 2025/0035640 A1
(43) Pub. Date: Jan. 30, 2025

(54) METHODS AND COMPOSITIONS FOR EDMAN-LIKE REACTIONS

(71) Applicant: BOARD OF REGENTS, THE UNIVERSITY OF TEXAS SYSTEM, Austin, TX (US)

(72) Inventors: Eric V. ANSLYN, Austin, TX (US); Harnimarta DEOL, Punjab (IN)

(21) Appl. No.: 18/682,238

(22) PCT Filed: Aug. 10, 2022
(86) PCT No.: PCT/US2022/074750

§ 371 Date: Feb. 8, 2024

Related U.S. Application Data
(60) Provisional application No. 63/232,139, filed on Aug. 11, 2021.

(51) Int. Cl. G01N 33/68; G01N 33/52; G01N 33/58

(52) U.S. Cl. CPC G01N 33/6824; G01N 33/52; G01N 33/582; G01N 2458/00

(57) ABSTRACT

Disclosed herein are reagents, compositions, methods, and systems for controlled terminal amino acid removal from peptides. Further disclosed are methods for identifying amino acids and sequences of peptides using the reagents, compositions, methods, and systems disclosed herein.

### CROSS-REFERENCE

This application claims the benefit of U.S. Provisional Application No. 63/232,139 filed Aug. 11, 2021, which application is incorporated herein by reference.

### BACKGROUND

The classic Edman degradation reaction using phenyl isothiocyanate in successive cycles to liberate individual amino acids in detectable phenylthiohydantoin forms via cleavage from a peptide has been used for over 50 years as a mainstay of protein sequencing. However, the traditional reagents (particularly harsh acid catalysis) used for this reaction make it difficult to adapt to new formats or chain with other reactions requiring more mild conditions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

There is need for compositions and methods that allow Edman-like sequencing of peptides under a wider range of conditions, particularly without harsh acid catalysis. Accordingly, provided herein are methods and compositions for Edman-like reactions for N-terminal sequencing operating under a wider range of reaction conditions than the traditional Edman reaction.

Various aspects of the present disclosure provide a method for analyzing a biomolecule comprising: (a) providing the biomolecule comprising a detectable label coupled to an amino acid of the biomolecule; (b) detecting a signal from the detectable label coupled to the amino acid of the biomolecule; (c) coupling an N-terminal coupling reagent to an N-terminal amino acid of the biomolecule to form a modified biomolecule, wherein the modified biomolecule comprises a hydroxamic acid or a hydrazide; and (d) subjecting the modified biomolecule to conditions sufficient to remove the N-terminal amino acid from the biomolecule.

In some embodiments, the detectable label is a dye. In some embodiments, the dye is a cyanine dye, diazo dye, organoboron dye, or a combination thereof. In some embodiments, the dye is a boron-dipyrromethane (BODIPY) dye. In some embodiments, the detectable label is a fluorescent label. In some embodiments, the biomolecule is a polypeptide. In some embodiments, the biomolecule is a protein.

In some embodiments, the detectable label generates at least one signal or at least one signal change. In some embodiments, the at least one signal or the at least one signal change is an optical signal. In some embodiments, the at least one signal or the at least one signal change comprises a plurality of signals of different intensities. In some embodiments, the at least one signal or the at least one signal change comprises a plurality of signals of different frequencies or signals of different frequency ranges. In some embodiments, the at least one signal or signal change is detected with an optical detector having single-molecule sensitivity.

In some embodiments, the detecting comprises fluorimetry. In some embodiments, the detecting comprises imaging. In some embodiments, the detecting identifies a sequence of the biomolecule. In some embodiments, the detectable label is coupled to an internal amino acid of the biomolecule. In some embodiments, the internal amino acid to which the detectable label couples is selected from the group consisting of cysteine, lysine, tyrosine, histidine, glutamate, aspartate, tryptophan, arginine, serine, threonine, and methionine. In some embodiments, the detectable label is an amino acid specific label. In some embodiments, the detectable label comprises a methionine specific label, an arginine specific label, a histidine specific label, a tyrosine specific label, a carboxylic acid R-group specific label, a lysine specific label, a cysteine specific label, a tryptophan specific label, or any combination thereof.

In some embodiments, the detectable label comprises at least two types of detectable labels, each of which couples to a different type of amino acid selected from the group consisting of cysteine, lysine, tyrosine, histidine, glutamate, aspartate, tryptophan, arginine, serine, threonine, and methionine. In some embodiments, the detectable label comprises at least three types of detectable labels, each of which couples to a different type of amino acid selected from the group consisting of cysteine, lysine, tyrosine, histidine, glutamate, aspartate, tryptophan, arginine, serine, threonine, and methionine. In some embodiments, the detectable label comprises at least four types of detectable labels, each of which couples to a different type of amino acid selected from the group consisting of cysteine, lysine, tyrosine, histidine, glutamate, aspartate, tryptophan, arginine, serine, threonine, and methionine. In some embodiments, the detectable label comprises at least five types of detectable labels, each of which couples to a different type of amino acid selected from the group consisting of cysteine, lysine, tyrosine, histidine, glutamate, aspartate, tryptophan, arginine, serine, threonine, and methionine.

In some embodiments, the amino acid specific label comprises a non-natural amino acid specific label. In some embodiments, the non-natural amino acid specific label is a phosphoserine specific label, phosphothreonine specific label, pyroglutamic acid specific label, hydroxyproline specific label, azidolysine specific label, or dehydroalanine specific label. In some embodiments, the amino acid to which the detectable label couples is a post-translationally modified amino acid. In some embodiments, the post-translationally modified amino acid is citrullinated, methylated, sulfurylated, phorphorylated, succinylated, glycosylated, palmitoylated, prenylated, acylated, amidated, hydroxylated, iodinated, chlorinated, fluorinated, nitrosylated, glutathionylated, malonated, biotinylated, oxidized, reduced, or any combination thereof.

In some embodiments, the modified biomolecule comprises a substituted hydroxamic acid. In some embodiments, the modified biomolecule comprises an unsubstituted hydroxamic acid. In some embodiments, removing the N-terminal amino acid from the biomolecule generates a 1,2,4-oxadiazinane-3,6-dione byproduct. In some embodiments, removing the N-terminal amino acid from the biomolecule generates a 5-substituted 1,2,4-oxadiazinane-3,6-dione byproduct. In some embodiments, the modified biomolecule comprises a hydrazide. In some embodiments, removing the N-terminal amino acid from the biomolecule generates a 1,2,4-triazine-3,6-dione byproduct. In some embodiments, removing the N-terminal amino acid from the biomolecule generates a 5-substituted 1,2,4-triazine-3,6-dione byproduct.

In some embodiments, the method comprises sequencing by degradation. In some embodiments, the N-terminal coupling reagent comprises a carbamate group. In some embodiments, the conditions to remove the N-terminal amino acid from the biomolecule comprises contacting the modified biomolecule with a base. In some embodiments, the base is a Ba(OH)₂. In some embodiments, the base is NaOH.

In some embodiments, the method further comprises immobilizing the biomolecule to a support. In some embodiments, the immobilizing comprises coupling a C-terminus of the biomolecule to the support. In some embodiments, the immobilizing comprises coupling a cysteine thiol of the biomolecule to the support. In some embodiments, the immobilizing comprises non-covalently coupling the biomolecule to a protein coupled to the support. In some embodiments, the protein comprises an antibody, a T-cell receptor, a pore protein, a catalytically inactive protease, or any combination thereof.

In some embodiments, the method further comprises repeating (a)-(d). In some embodiments, the method further comprises identifying an unlabeled amino acid of the biomolecule. In some embodiments, the at least one amino acid removed from the modified biomolecule comprises the N-terminal amino acid.

In some embodiments, the N-terminal coupling agent is a compound of Formula (II), or a salt, a solvate, or a derivative thereof:

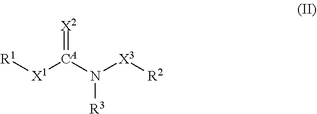

wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen,
wherein the reagent modifies the N-terminal amino acid of the peptide.

In some embodiments, R¹ is an electron withdrawing group. In some embodiments, R¹ is an electron donating group. In some embodiments, R¹ is amino, alkoxy, aryl, or heteroaryl, each of which is independently unsubstituted or substituted; or hydroxy. In some embodiments, R¹ is substituted phenyl. In some embodiments, R¹ is nitrophenyl.

In some embodiments, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group. In some embodiments, R² comprises a silyl group. In some embodiments, the silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM). In some embodiments, R² is tert-butyldimethylsilyl. In some embodiments, R² is trimethylsilyl.

In some embodiments, X¹ is O. In some embodiments, X² is O. In some embodiments, X³ is O.

In some embodiments, each R³ and R⁴ is independently C₁-C₉ alkyl, C₁-C₉ alkenyl, or C₁-C₉ alkynyl, each of which is independently unsubstituted or substituted. In some embodiments, R³ is hydrogen. In some embodiments, R⁴ is hydrogen.

In some embodiments, the reagent has the structure:

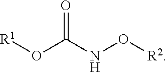

Various aspects of the present disclosure provide a composition comprising: (a) a peptide comprising an N-terminal amino acid; and (b) a reagent comprising a structure of Formula (II), or a salt, a solvate, or a derivative thereof:

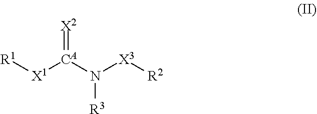

wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen,
wherein the reagent modifies the N-terminal amino acid of the peptide.

In some embodiments, R¹ is an electron withdrawing group. In some embodiments, R¹ is an electron donating group. In some embodiments, R¹ is amino, alkoxy, aryl, or heteroaryl, each of which is independently unsubstituted or substituted; or hydroxy. In some embodiments, R¹ is substituted phenyl. In some embodiments, R¹ is nitrophenyl.

In some embodiments, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group. In some embodiments, R² comprises a silyl group. In some embodiments, the silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM). In some embodiments, R² is tert-butyldimethylsilyl. In some embodiments, R² is trimethylsilyl.

In some embodiments, X¹ is O. In some embodiments, X² is O. In some embodiments, X³ is O.

In some embodiments, each R³ and R⁴ is independently C₁-C₉ alkyl, C₁-C₉ alkenyl, or C₁-C₉ alkynyl, each of which is independently unsubstituted or substituted. In some embodiments, R³ is hydrogen. In some embodiments, R⁴ is hydrogen. In some embodiments, the reagent has the structure:

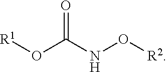

In some embodiments, the method further comprises an organic solvent. In some embodiments, the organic solvent is dimethylsulfoxide (DMSO). In some embodiments, the organic solvent is dimethylformamide (DMF). In some embodiments, R² is configured for cleavage by a base. In some embodiments, the base is a halide. In some embodiments, the halide is fluoride. In some embodiments, the reagent is configured to cleave the N-terminal amino acid from the peptide.

Various aspects of the present disclosure provide a method comprising: (a) providing a polypeptide immobilized to a support, wherein the polypeptide comprises at least one labeled internal amino acid; (b) detecting at least one signal or signal change from the polypeptide immobilized to the support to identify at least a portion of a sequence of the polypeptide; and (c) subjecting the polypeptide to conditions sufficient to remove at least one amino acid from the polypeptide to form a cleaved polypeptide via a hydroxamic acid or a hydrazide intermediate.

Various aspects of the present disclosure provide a composition for modifying an amine of an N-terminal amino acid of a peptide said composition comprising a reagent comprising a structure of Formula (I), or a salt, a solvate, or a derivative thereof:

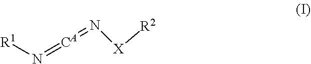

wherein:
R¹ is an electron donating group or an electron withdrawing group;
R² is a protecting group configured for cleavage from said reagent;
X is O, S, Se, or NR⁴; and
R⁴ is hydrogen, optionally substituted alkyl, optionally substituted alkenyl, or optionally substituted alkynyl. In embodiments, said reagent is configured to couple to an internal atom of said peptide upon or subsequent to said cleavage.

In some embodiments, X is O or S. In some embodiments, X is S. In some embodiments, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, a sulfonamide group, or any combination thereof. In some embodiments, R² comprises a silyl group. In some embodiments, said silyl group comprises trimethylsilyl (TMS), triethylsilyl (TES), tert-butyldimethylsilyl (TBDMS), tert-Butyldiphenylsilyl (TBDPS), triisopropylsilyl (TIPS), triisopropylsilyloxymethyl (TOM), or any combination thereof. In some embodiments, said silyl group comprises trimethylsilyl (TMS). In some embodiments, R⁴ comprises optionally substituted C₁-C₉ alkyl, optionally substituted C₁-C₉ alkenyl, or optionally substituted C₁-C₉ alkynyl.

In some embodiments, R³ is hydrogen. In some embodiments, C^{A} of said reagent is configured to couple said N-terminal amine of said peptide prior to R² cleavage. In some embodiments, C^{A} of said reagent is configured to not couple to said N-terminal amine of said peptide subsequent to R² cleavage. In some embodiments, R² is configured for cleavage by a base. In some embodiments, said base is a halide. In some embodiments, said halide is fluoride. In some embodiments, said cleavage requires temperatures above 25° C. In some embodiments, said cleavage may be performed in neutral or alkaline organic media. In some embodiments, said cleavage may be performed in alkaline organic media.

In some embodiments, subsequent to said cleavage, said reagent comprises an oxime or an oximate. In some embodiments, said oxime or said oximate is configured to couple to said internal amino acid of said peptide. In some embodiments, said reagent is configured to cleave said N-terminal amino acid from said peptide upon or subsequent to said coupling to said internal atom of said peptide. In some embodiments, said cleaving said N-terminal amino acid from said peptide generates a 3-imino1,2,4-oxadiazinanone. In some embodiments, said internal atom is a carbonyl carbon of said peptide. In some embodiments, said internal atom is a carbonyl carbon of the N-terminal amino acid of said peptide.

In some embodiments, R¹ comprises an optionally substituted alkyl group, an optionally substituted aryl group, or an optionally substituted heteroaryl group. In some embodiments, R¹ comprises a detectable moiety. In some embodiments, said detectable moiety comprises a fluorescent dye, an electrochemically detectable moiety, or a mass tag.

In some embodiments, said composition comprises an organic solution comprising said reagent comprising a structure of Formula (I), or said salt, said solvate, or said derivative thereof. In some embodiments, said reagent comprises a dimethylsulfoxide (DMSO) solubility of about 10 mg/mL to about 1 μg/mL. In some embodiments, said reagent comprises a half-life of about 1 day to 10 years when stored in dry form, in dry conditions, at 25° C., and in the absence of light. In some embodiments, said reagent comprises a half-life of about 1 day to 10 years when stored in dimethylsulfoxide (DMSO) at 25° C. in the absence of light.

Various aspects of the present disclosure provide a composition for modifying an amine of an N-terminal amino acid of a peptide, said composition comprising a reagent comprising a structure of Formula (II), or a salt, a solvate, or a derivative thereof:

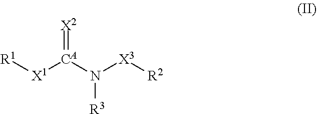

wherein:
R¹ is an electron donating group or an electron withdrawing group;
R² is a leaving group configured for cleavage from said reagent;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴; and
each instance of R³ and R⁴ is independently selected from the group consisting of hydrogen, optionally substituted alkyl, optionally substituted alkenyl, and optionally substituted alkynyl. In some embodiments, said reagent is configured to couple to an internal atom of said peptide upon or subsequent to said cleavage.

In some embodiments, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, a sulfonamide group, or any combination thereof. In some embodiments, R² comprises a silyl group. In some embodiments, said silyl group comprises trimethylsilyl (TMS), triethylsilyl (TES), tert-butyldimethylsilyl (TBDMS), tert-Butyldiphenylsilyl (TBDPS), triisopropylsilyl (TIPS), triisopropylsilyloxymethyl (TOM), or any combination thereof. In some embodiments, said silyl group comprises trimethylsilyl (TMS).

In some embodiments, X¹ and X² are each independently selected from O and NR⁴. In some embodiments, X¹ is NR⁴ and X² is O. In some embodiments, X³ is O. In some embodiments, each instance of R³ is hydrogen. In some embodiments, C^{A} of said reagent is configured to couple to said N-terminal amine of said peptide prior to R² cleavage. In some embodiments, C^{A} of said reagent is configured to not couple to said N-terminal amine of said peptide subsequent to R² cleavage. In some embodiments, said coupling to said N-terminal amine of said peptide results in loss of R¹—X¹ from said reagent. In some embodiments, nucleophilic substitution at C^{A} favors a loss of R¹—X¹ over a loss of (NR³)—O—R².

In some embodiments, R² is configured for cleavage by a base. In some embodiments, said base is a halide. In some embodiments, said halide is fluoride. In some embodiments, said cleavage requires temperatures above 25° C.

In some embodiments, R³ or R⁴ comprises an optionally substituted C₁-C₉ alkyl, an optionally substituted C₁-C₉ alkenyl, or an optionally substituted C₁-C₉ alkynyl. In some embodiments, said cleavage may be performed in neutral or alkaline organic solution. In some embodiments, said cleavage may be performed in alkaline organic solution. In some embodiments, subsequent to said cleavage, said reagent comprises an oxime or an oximate. In some embodiments, said oxime or said oximate is configured to couple to said internal amino acid of said peptide.

In some embodiments, said reagent is configured to cleave said N-terminal amino acid from said peptide upon or subsequent to said coupling to said internal atom of said peptide. In some embodiments, said internal atom is a carbonyl carbon of said peptide. In some embodiments, R³ comprises a detectable moiety. In some embodiments, said detectable moiety comprises a fluorescent dye, an electrochemically detectable moiety, or a mass tag.

In some embodiments, said composition comprises a dimethylsulfoxide (DMSO) solution comprising said reagent comprising a structure of Formula (II), or said salt, said solvate, or said derivative thereof. In some embodiments, said reagent comprises a DMSO solubility of about 10 mg/mL to about 1 μg/mL. In some embodiments, said reagent comprises a half-life of about 1 day to 10 years when stored in dry form, in dry conditions, at 25° C., and in the absence of light. In some embodiments, said reagent comprises a half-life of about 1 day to 10 years when stored in dimethylsulfoxide (DMSO), at 25° C., and in the absence of light.

Various aspects of the present disclosure provide a method for analyzing a peptide comprising an N-terminal amino acid, said method comprising: (a) coupling a detectable label to an amino acid of said peptide, wherein said detectable label comprises a specificity for the side chain of said amino acid; (b) detecting a signal from said detectable label coupled to said peptide; (c) coupling an N-terminal coupling reagent to said N-terminal amino acid of said peptide; and (d) cleaving said N-terminal coupling reagent, thereby activating said N-terminal coupling agent to remove at least one amino acid from said peptide.

In some embodiments, comprising immobilizing said peptide to a support. In some embodiments, said immobilizing is after (a) and before (b). In some embodiments, said immobilizing comprises coupling a C-terminus of said peptide to said support. In some embodiments, said immobilizing comprises coupling a cysteine thiol of said peptide to said support. In some embodiments, immobilizing comprises non-covalently coupling said peptide to a protein coupled to said support. In some embodiments, said protein comprises an antibody, a T-cell receptor, a pore protein, a catalytically inactive protease, or any combination thereof.

In some embodiments, said amino acid to which said detectable label couples is selected from the group consisting of cysteine, lysine, tyrosine, histidine, glutamate, aspartate, tryptophan, arginine, serine, threonine, and methionine. In some embodiments, said detectable label comprises at least two types of detectable labels, each of which couples to a different type of amino acid selected from the group consisting of cysteine, lysine, tyrosine, histidine, glutamate, aspartate, tryptophan, arginine, serine, threonine, and methionine. In some embodiments, said detectable label comprises at least three types of detectable labels, each of which couples to a different type of amino acid selected from the group consisting of cysteine, lysine, tyrosine, histidine, glutamate, aspartate, tryptophan, arginine, serine, threonine, and methionine. In some embodiments, said detectable label comprises at least four types of detectable labels, each of which couples to a different type of amino acid selected from the group consisting of cysteine, lysine, tyrosine, histidine, glutamate, aspartate, tryptophan, arginine, serine, threonine, and methionine. In some embodiments, said detectable label comprises at least five types of detectable labels, each of which couples to a different type of amino acid selected from the group consisting of cysteine, lysine, tyrosine, histidine, glutamate, aspartate, tryptophan, arginine, serine, threonine, and methionine. In some embodiments, said amino acid to which said detectable label couples is a post-translationally modified amino acid. In some embodiments, said post-translationally modified amino acid is citrullinated, methylated, sulfurylated, phorphorylated, succinylated, glycosylated, palmitoylated, prenylated, acylated, amidated, hydroxylated, iodinated, chlorinated, fluorinated, nitrosylated, glutathionylated, malonated, biotinylated, oxidized, reduced, or any combination thereof. In some embodiments, said signal comprises a fluorescence signal.

In some embodiments, said N-terminal coupling reagent comprises a carbodiimide or a urea moiety. In some embodiments, said cleaving said N-terminal coupling reagent generates an oxime or an oximate. In some embodiments, said removing said at least one amino acid comprises coupling between said oxime or said oximate and a carbonyl of said peptide.

In some embodiments, said cleaving said N-terminal coupling reagent comprises an acidic pH. In some embodiments, said cleaving said N-terminal coupling reagent comprises a neutral or alkaline pH. In some embodiments, said cleaving said N-terminal coupling reagent comprises conditions in which a dye selected from the group consisting of an organoboron dye, a diazo dye, a cyanine dye, or any combination thereof. In some embodiments, said cleaving comprises a base. In some embodiments, said base is a halide. In some embodiments, said base is fluoride.

In some embodiments, said coupling said N-terminal coupling reagent to said N-terminal amino acid of said peptide and said cleaving said N-terminal coupling reagent are performed in less than 30 minutes. In some embodiments, said coupling said N-terminal coupling reagent to said N-terminal amino acid of said peptide and said cleaving said N-terminal coupling reagent are performed in less than 10 minutes.

In some embodiments, the method further comprises repeating (b)-(d) at least once. In some embodiments, the method further comprises identifying an unlabeled amino acid of said peptide. In some embodiments, the method further comprises identifying an amino acid sequence of said peptide. In some embodiments, the method further comprises detecting a signal from said N-terminal coupling reagent subsequent to said coupling said N-terminal coupling reagent to said N-terminal amino acid of said peptide.

In some embodiments, said at least one amino acid removed from said peptide comprises said N-terminal amino acid. In some embodiments, prior to said coupling said N-terminal coupling reagent to said N-terminal amino acid of said peptide said method comprises activating said N-terminal coupling reagent for said coupling to said N-terminal amino acid of said peptide. In some embodiments, said activating comprises alkylating said N-terminal coupling reagent.

Another aspect of the present disclosure provides a non-transitory computer readable medium comprising machine executable code that, upon execution by one or more computer processors, implements an of the methods above or elsewhere herein.

Another aspect of the present disclosure provides a system comprising one or more computer processors and/or computer memory coupled thereto. The computer memory comprises machine executable code that, upon execution by one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein.

FIG. 2A provides a reaction scheme for N-terminal amino acid removal with a reagent comprising a structure of Formula (I). FIG. 2B provides an example of N-terminal amino acid removal with a carbodiimide reagent comprising a structure of Formula (I).

FIG. 3A provides a reaction scheme for N-terminal amino acid removal with a reagent comprising a structure of Formula (II). FIG. 3B provides an example of N-terminal amino acid removal with a carbamate reagent comprising a structure of Formula (II).

FIG. 4A-4B show that sequencing of a functionalized peptide with a phenylalanine at the N-terminus produced the desired degraded peptide product after treatment with Tris buffer (pH 9.5) at 50° C. for 2 h.

FIG. 5 shows LC-MS analysis of a peptide comprising an N-terminal tyrosine upon treatment with 2% NaOH at 50° C. for 5 h.

FIG. 6A shows an LC-MS trace of a functionalized peptide (YGFWVY; SEQ ID NO: 1) with a tyrosine residue at the N-terminal before cleavage with Ba(OH)₂. FIG. 6B shows LC-MS data of the degraded peptide after treatment with 1% Ba(OH)₂ at 37° C. for 5 hr, including: 1) complex converted into side product; 2) degraded peptide; and 3) cyclized product.

FIG. 7A shows an LC-MS trace of a functionalized peptide (YGFWVY; SEQ ID NO: 1) before cleavage with Ba(OH)₂. FIG. 7B shows LC-MS data of the degraded peptide after treatment with 1% Ba(OH)₂ at 37° C. for 5 hr, including: 1) complex converted into side product; 2) degraded peptide; and 3) cyclized product.

FIG. 8A shows an LC-MS trace of a functionalized peptide (WYFWYV; SEQ ID NO: 2) with a tryptophan residue at the N-terminal before cleavage with Ba(OH)₂. FIG. 8B shows LC-MS data of the degraded peptide after treatment with 1% Ba(OH)₂ at 37° C. for 5 hr, including: 1) complex converted into side product; 2) degraded peptide; 3) cyclized product; and 4) unreacted product.

FIG. 9A shows an LC-MS trace of a functionalized peptide (KYFW; SEQ ID NO: 9) with a lysine residue at the N-terminal before cleavage with Ba(OH)₂. FIG. 9B shows LC-MS data of the degraded peptide after treatment with 1% Ba(OH)₂ at 37° C. for 5 hr, including: 1) complex converted into side product; 2) degraded peptide; and 3) an impurity.

FIG. 10A shows an LC-MS trace of a functionalized peptide (CYFWVY; SEQ ID NO: 7) with a cysteine residue at the N-terminal before cleavage with Ba(OH)₂. FIG. 10B shows LC-MS data of the degraded peptide after treatment with 1% Ba(OH)₂ at 37° C. for 5 hr, including: 1) complex converted into side product; 2) degraded peptide; and 3) unreacted product.

FIG. 11A shows an LC-MS trace of a functionalized peptide (HYFWVY; SEQ ID NO: 6) with a histidine residue at the N-terminal before cleavage with Ba(OH)₂. FIG. 11B shows LC-MS data of the degraded peptide after treatment with 1% Ba(OH)₂ at 37° C. for 5 hr, including: 1) complex converted into side product; and 2) degraded peptide.

FIG. 12A shows an LC-MS trace of a functionalized peptide (EYFWVY; SEQ ID NO: 4) with a glutamic acid residue at the N-terminal before cleavage with Ba(OH)₂. FIG. 12B shows LC-MS data of the degraded peptide after treatment with 1% Ba(OH)₂ at 37° C. for 5 hr, including: 1) complex converted into side product; 2) degraded peptide; and 3) unreacted peptide.

FIG. 13A shows an LC-MS trace of a functionalized peptide (DYFWVY; SEQ ID NO: 5) with a aspartic acid residue at the N-terminal before cleavage with Ba(OH)₂. FIG. 13B shows LC-MS data of the degraded peptide after treatment with 1% Ba(OH)₂ at 37° C. for 5 hr, including: 1) complex converted into side product; and 2) degraded peptide.

FIG. 14A shows an LC-MS trace of a functionalized peptide (PFFAYV; SEQ ID NO: 10) with a proline residue at the N-terminal before cleavage with Ba(OH)₂. FIG. 14B shows LC-MS data of the degraded peptide after treatment with 1% Ba(OH)₂ at 37° C. for 5 hr, including: 1) complex converted into side product; 2) degraded peptide; 3) original peptide; and 4) TBAF.

FIG. 15A shows an LC-MS trace of a functionalized peptide (SYFWVY; SEQ ID NO: 8) with a serine residue at the N-terminal before cleavage with Ba(OH)₂. FIG. 15B shows LC-MS data of the degraded peptide after treatment with 1% Ba(OH)₂ at 37° C. for 5 hr, including: 1) complex converted into side product; 2) degraded peptide; 3) TBAF.

FIG. 16A shows an LC-MS trace prior to treatment with Ba(OH)₂. FIG. 16B shows an LC-MS trace after treatment with Ba(OH)₂.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “leaving group” generally refers to an atom or group that is cleaved under the conditions of a substitution reaction. Examples of leaving groups include, but are not limited to, halogen, alkane or arylene sulfonyloxy such as methanesulfonyloxy, ethanesulfonyloxy, thiomethyl, benzenesulfonyloxy, tosyloxy and thienyloxy; dihalogenophosphinoyloxy optionally substituted with benzyloxy, isopropyloxy, acyloxy and the like. In some embodiments, the leaving group may be HC(O)—COOH or RC(O)—COOH, where R is C₁-C₆ C₆ alkyl or substituted C₁-C₆ alkyl.

As used herein the term “protecting group”, generally refers to a labile chemical moiety which protects reactive groups including without limitation, hydroxyl, amino and thiol groups, against undesired reactions. Protecting groups are typically used selectively and/or orthogonally to protect sites during reactions at other reactive sites and can then be removed to leave the unprotected group as is or available for further reactions. Protecting groups as known in the art are described generally in Greene's Protective Groups in Organic Synthesis, 4th edition, John Wiley & Sons, New York, 2007. Protecting groups include, e.g. silyl groups such as trimethylsilyl (TMS), triethylsilyl (TES), tert-butyldimethylsilyl (TBDMS), tert-Butyldiphenylsilyl (TBDPS), triisopropylsilyl (TIPS), triisopropylsilyloxymethyl (TOM), or any combination thereof. Protecting groups also include, e.g., an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, a sulfonamide group, or any combination thereof.

As used herein, the term “electron withdrawing group” generally refers to a group that withdraws electron density, such as, for example, from the pi-system of the indeno-fused naphthopyran core structure, or through the sigma-system of a haloalkyl compound. In some cases, an “electron withdrawing group”, as used herein, can be defined as a group having a positive Hammett Op value, when the group is attached to a carbon participating in an aromatic pi-system, such as the aromatic pi-system of the indeno-fused naphthopyran core. The term “Hammett Op value” generally refers to a measurement of the electronic influence, as either an electron-donating or electron-withdrawing influence, of a substituent attached to a carbon participating in an aromatic pi system that is transmitted through the polarizable pi electron system, such as, for example, an aromatic pi electron system. The Hammett Op value is a relative measurement comparing the electronic influence of the substituent in the para position of a phenyl ring to the electronic influence of a hydrogen substituted at the para position. In many cases, for aromatic substituents, a negative Hammett Op value indicates that a group or substituent donates electron density to another portion of a molecule, while (e.g., acts as an electron-donating group) a positive Hammett Op value indicates that a group or substituent withdraws electron density from another portion of a molecule (e.g., acts as an electron-withdrawing group). In some cases, Electron-withdrawing groups suitable for use in connection with embodiments of the disclosure may have a Hammett Op value ranging from about 0.05 to about 0.75. Suitable electron-withdrawing groups may comprise, for example: halogen, such as fluoro (σₚ=0.06), chloro (σₚ=0.23), and bromo (σₚ=0.23); perfluoroalkyl (for example, —CF₃, σₚ=0.54) or perfluoroalkoxy (for example, —OCF₃, σₚ=0.35). Further suitable electron-withdrawing substituents having Hammett op values in the range from about 0.05 to about 0.75 are set forth in “Section 9 Physicochemical Relationships” in Lange's Handbook of Chemistry, 15^{th} ed. J. A. Dean, editor, McGraw Hill, 1999, pp 9.1-9.8, the disclosure of which is incorporated herein by reference. In some cases, when referring to the Hammett σ value, the subscript “p”, refers to the Hammett σₚ value as measured when the group is located at the para position of a phenyl ring of a model system, such as a para-substituted benzoic acid model system.

As used herein, the term “electron donating group” generally refers to a group that increases electron density in another portion of a molecule, such as, for example, an alkylamino substituent which donates electron density into an aromatic system. Examples of an “electron-donating group” can include an atom bonded directly to a pi-system of the photochromic material, wherein the atom has at least one lone pair of electrons which are capable of resonance into the pi system of the aromatic ring structure, and/or the group may donate electron density into the pi system by a hyperconjugative effect, such as, for example, an alkyl substituent. In some cases, an “electron donating group”, as used herein, can be defined as a group having a negative Hammett Op value, when the group is attached to a carbon participating in an aromatic pi system. Example electron donating groups for use with methods and compositions according to the present disclosure include e.g. vinyl, aryl, heteroaryl, amine, alkoxy, and alkyl groups.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₈ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more substituents such as those substituents described herein.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents such as those substituents described herein.

Included in the present disclosure are salts, including pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

As used herein, the term “pharmaceutically acceptable salt” generally refers to those salts which are suitable for use in contact with the tissues of subjects without e.g. undue toxicity, irritation or allergic response and are commensurate with e.g. a reasonable benefit/risk ratio. Pharmaceutically acceptable salts have been described elsewhere. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Inorganic acids from which salts can be derived include, but are not limited to, hydrochloric acid, hydrobromic acid, sulfuric acid, nitric acid, and phosphoric acid. Organic acids from which salts can be derived include, but are not limited to, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Pharmaceutically acceptable salts derived from appropriate bases include alkali metal, alkaline earth metal, ammonium and N⁺(C₁₋₄alkyl)⁴-salts. Inorganic bases from which salts can be derived include, but are not limited to, sodium, potassium, lithium, ammonium, calcium, magnesium, iron, zinc, copper, manganese, aluminum, and the like. Organic bases from which salts can be derived include, but are not limited to, primary, secondary, and tertiary amines, substituted amines, including naturally occurring substituted amines, cyclic amines, basic ion exchange resins, and the like, examples include, but are not limited to, isopropylamine, trimethylamine, diethylamine, triethylamine, tripropylamine, and ethanolamine. In some embodiments, the pharmaceutically acceptable base addition salt is ammonium, potassium, sodium, calcium, or magnesium salts. Representative alkali or alkaline earth metal salts include sodium, lithium, potassium, calcium, magnesium, iron, zinc, copper, manganese, and aluminum. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (e.g., two counterions) and higher salts (e.g., three or more counterions) are encompassed within the meaning of pharmaceutically acceptable salts.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N (R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN (R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N (R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain.

In some embodiments, substituents can include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N (R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN (R^{a})₂ (where t is 1 or 2); and alkyl, alkenyl, and alkynyl each of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where tis 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain.

In some embodiments, substituents can include any substituents described herein, for example: halogen, haloalkyl, oxo (═O), hydroxy, thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN (R^{a})₂ (where t is 1 or 2); and alkenyl, alkynyl, aryl, aralkyl, aralkenyl, aralkynyl, cycloalkyl, cycloalkylalkyl, heterocycloalkyl, heterocycloalkylalkyl, heteroaryl, heteroarylalkyl, wherein the alkenyl, alkynyl, haloalkyl, haloalkenyl, haloalkynyl, aryl, aralkyl, aralkenyl, aralkynyl, cycloalkyl, cycloalkylalkyl, heterocycloalkyl, heterocycloalkylalkyl, heteroaryl, and heteroarylalkyl each of which may be optionally substituted by alkyl, alkenyl, alkynyl, halogen, haloalkyl, haloalkenyl, haloalkynyl, hydroxy, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, hydroxy, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain.

In some embodiments, substituents can include any substituents described herein, for example: halogen, hydroxy, fluoroalkyl, oxo (═O), cyano (—CN), nitro (—NO₂), —R^{b}—OR^{a}, —R^{b}—N(R^{a})₂, —R^{b}—C(O)R^{a}, —R^{b}—C(O)OR^{a}, —R^{b}—C(O)N(R^{a})₂, and —R^{b}—N(R^{a})C(O)R^{a}; and alkyl, aryl, cycloalkyl, heterocycloalkyl, and heteroaryl, each of which may be optionally substituted by alkyl, alkenyl, alkynyl, halogen, haloalkyl, haloalkenyl, haloalkynyl, oxo (═O), hydroxy, thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N (R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain.

In some embodiments, substituents can include any substituents described herein, for example: alkyl, halo, fluoroalkyl, oxo (═O), hydroxy, cyano (—CN), —R^{b}—OR^{a}, —R^{b}—N(R^{a})₂, —R^{b}—C(O)R^{a}, and —R^{b}—C(O)OR^{a}, wherein the alkyl may be optionally substituted by alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O) R^{a} (where tis 1 or 2), —R^{b}—S(O)ₜR^{a} (where t is 1 or 2), —R^{b}—S(O)ₜOR^{a} (where t is 1 or 2) and —R^{b}—S(O)ₜN (R^{a})₂ (where t is 1 or 2); and wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N (R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain.

In addition, if a compound of the present disclosure is obtained as an acid addition salt, the free base can be obtained by basifying a solution of the acid salt. Conversely, if a product is a free base, an acid addition salt, particularly a pharmaceutically acceptable addition salt, can be produced by dissolving the free base in a suitable organic solvent and treating the solution with an acid, in accordance with conventional procedures for preparing acid addition salts from base compounds.

As used herein, the term “solvate” generally refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, 3 or 4, solvent or water molecules. In some embodiments, the solvate can be a channel solvate. It will be understood that the term “compound” as used herein encompasses the compound and solvates of the compound, as well as mixtures thereof.

The term “analyte” or “analytes,” as used herein, generally refers to a molecule whose presence or absence is measured or identified. An analyte can be a molecule for which a detectable probe or assay exists or can be produced. For example, an analyte can be a macromolecule, such as, for example, a nucleic acid, a polypeptide, a carbohydrate, a small organic, an inorganic compound, or an element, for example, gold, iron, or lead. An analyte can be part of a sample that contains other components, or can be the sole or the major component of the sample. An analyte can be a component of a whole cell or tissue, a cell or tissue extract, a fractionated lysate thereof or a substantially purified molecule. In some embodiments, the target analyte is a polypeptide.

The terms “polypeptide” and “peptide” generally to refer to a polymer of amino acids in which an amino acid may be linked to another amino acid by a peptide bond. In some examples, a polypeptide is a protein. The amino acid may be a naturally occurring amino acid or a non-naturally occurring amino acid (i.e., amino acid analogue). The polymer can be linear or branched and can include modified amino acids, and/or may be interrupted by non-amino acids. Polypeptides can occur as single chains or associated chains. The polymer may include a plurality of amino acids and may have a secondary and tertiary structure (i.e., protein). In some examples, the polymer comprises at least about 2, at least about 3, at least about 4, at least about 5, at least about 6, at least about 7, at least about 8, at least about 9, at least about 10, at least about 20, at least about 30, at least about 40, at least about 50, at least about 100, at least about 1000, at least about 10,000, or more amino acids.

The term “amino acid,” as used herein, generally refers to a naturally occurring or non-naturally occurring amino acid (amino acid analogue). The non-naturally occurring amino acid may be a synthesized amino acid. As used herein, the terms “amino acid sequence,” “peptide sequence,” and “polypeptide sequence,” as used herein, generally refer to at least two amino acids or amino acid analogs that are covalently linked by a peptide (amide) bond or an analog of a peptide bond. The term peptide includes oligomers and polymers of amino acids or amino acid analogs. The amino acids of the peptide may be L-amino acids or D-amino acids. A peptide, polypeptide, or protein may be synthetic, recombinant, or naturally occurring. A synthetic peptide may be a peptide that is produced by artificial approaches in vitro.

The terms “amino acid sequence,” “peptide sequence,” and “polypeptide sequence,” as used herein, generally refer to a sequence of at least two amino acids or amino acid analogs that are covalently linked (e.g., by a peptide (amide) bond or an analog of a peptide bond). A peptide sequence may refer to a complete sequence or a portion of a sequence. For example, a peptide sequence may contain gaps, positions with unknown identities, or positions that can accommodate distinct species.

As used herein, the term “side chain” or “R-group” generally refers to structures attached to an amino acid alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−), and glutamate (−): amino acids can also be basic, such as lysine, or acidic, such as glutamic acid; uncharged polar side chains have hydroxyl, amide, or thiol groups, such as cysteine having a chemically reactive side chain, i.e., a thiol group that can form bonds with another cysteine, serine (Ser) and threonine (Thr), that have hydroxylic R side chains of different sizes; asparagine (Asn), glutamine (Gln), and tyrosine (Tyr); non-polar hydrophobic amino acid side chains include the amino acid glycine, alanine, valine, leucine, and isoleucine having aliphatic hydrocarbon side chains ranging in size from a methyl group for alanine to isomeric butyl groups for leucine and isoleucine; methionine (Met) has a thiol ether side chain; proline (Pro) has a cyclic pyrrolidine side group. Phenylalanine (with its phenyl moiety) (Phe) and tryptophan (Trp) (with its indole group) contain aromatic side chains, which are characterized by bulk as well as lack of polarity.

The term “cleavable unit,” as used herein, generally refers to a molecule that can be split into at least two molecules. Non-limiting examples of cleavage reagents and conditions to split a cleavable unit include: enzymes, nucleophilic or basic reagents, reducing agents, photo-irradiation, electrophilic or acidic reagents, organometallic or metal reagents, and oxidizing reagents.

The term “sample,” as used herein, generally refers to a sample containing or suspected of containing a polypeptide. For example, a sample can be a biological sample containing one or more polypeptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the polypeptides and/or to include reagents.

As used herein, the term “support” generally refers to an entity to which a substance (e.g., molecular construct) can be coupled, immobilized, or adsorbed. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, a lantern, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane. The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine, or asparagine). The functional group may couple specifically to a reactive group on a particular species, such as a label. In some examples of functionalized beads, the functional motif can be reversibly coupled and cleaved. A functional motif can also irreversibly couple to a molecule.

One such type of substrate is a lantern, which may comprise a solid support comprising peptide capture agents, and a rod for positioning the solid support within a sample. A lantern rod may be manipulatable by a user (e.g., the user may hold the lantern rod) or an instrument. A lantern rod may be configured to connect to a member proximal to a sample volume. For example, a lantern rod may be configured to couple to a clip above a well of a well plate. A lantern solid support may comprise a reactive group of the present disclosure, such as a reactive group selective for cysteine or a peptide C-terminus. A lantern may be dried or frozen with peptides coupled to its solid support, which may stabilize the peptides coupled thereto. Unbound peptide may be washed from a lantern solid support.

As used herein, sequencing of peptides “at the single molecule level” generally refers to amino acid sequence information obtained from individual (i.e., single) peptide molecules in a mixture of diverse peptide molecules. The amino acid sequence information may be obtained from an entirety of an individual peptide molecule or one or more portion of the individual peptide molecule, such as a contiguous amino acid sequence of at least a portion of the individual peptide molecule. Alternatively, partial amino acid sequence information may be obtained, which may allow for identification of the peptide or protein. Partial amino acid sequence information, including for example, the pattern of a specific amino acid residue (i.e., lysine) within individual peptide molecules, may be sufficient to uniquely identify an individual peptide molecule. For example, a pattern of amino acids may comprise a plurality of identified positions (e.g., identified as a particular amino acid type, such as lysine, or identified as a particular set of amino acids, such as the set of carboxylate side chain-containing amino acids), and a plurality of unidentified positions. The sequence of identified positions may be searched against a known proteome of a given organism to identify the individual peptide molecule. In some examples, sequencing of a peptide at the single molecule level may identify a pattern of a certain type of amino acid (e.g., lysine) in an individual peptide molecule. Such information may be used to identify a macromolecule (e.g., protein) from which the peptide was derived. This may advantageously preclude the need to identify all amino acids of the peptide.

As used herein, the term “Edman degradation” generally refers to methods comprising chemical removal of amino acids from peptides or proteins. In some cases, Edman degradation denotes terminal (e.g., N- or C-terminal) amino acid removal. In specific cases, Edman degradation refers to N-terminal amino acid removal through isothiocyanate (e.g., phenyl isothiocyanate) coupling and cyclization with the terminal amine group of an N-terminal residue, such that the N-terminal amino acid is removed from a peptide. In some cases, Edman degradation refers to N-terminal amino acid removal through use of any of the Edman reagents described herein in place of isothiocyanate (e.g. a compound of Formula I, a compound of Formula II, or any combination thereof). In some cases, Edman degradation broadly encompasses N-terminal amino acid functionalizations leading to N-terminal amino acid removal. In some cases, Edman degradation encompasses C-terminal amino acid removal. In some cases, Edman degradation comprises terminal amino acid functionalization (e.g., N-terminal amino acid isothiocyanate functionalization) followed by enzymatic removal (e.g., by an ‘Edmanase’ with specificity for chemically derivatized N-terminal amino acids).

As used herein, the term “single molecule sensitivity” generally refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). This may include the ability to simultaneously record the fluorescent intensity of multiple individual (i.e., single) peptide molecules distributed across the glass surface. Optical devices are commercially available that can be applied in this manner. For example, a conventional microscope equipped with total internal reflection illumination and an intensified charge-couple device (CCD) detector is available. Imaging with a high sensitivity CCD camera allows the instrument to simultaneously record the fluorescent intensity of multiple individual (i.e., single) peptide molecules distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single polypeptide having a particular sequence or a site can include several polypeptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and an well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group, such as an amino acid side chain, C-terminal carboxylate, or N-terminal amine. The label may comprise at least one reactive group (e.g., a first reactive group and a second reactive group). The at least one reactive group may be configured to couple to a polypeptide. The at least one reactive group may be configured to couple to a support. The at least one reactive group may be coupled to or configured to couple to a detectable moiety. A label may provide a measurable signal.

As used herein, the term “polymer matrix” generally refers to a continuous phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid or gaseous species. For example, the term ‘polymer matrix’ may encompass desiccated hydrogels, hydrated hydrogels, and hydrogels containing glass fibers.

Peptide sequence information may be obtained from a polypeptide molecule or from one or more portions of the polypeptide molecule. Peptide sequencing may provide complete or partial amino acid sequence information for a peptide sequence or a portion of a peptide sequence. At least a portion of the peptide sequence may be determined at the single molecule level. In some cases, partial amino acid sequence information, including for example, the relative positions of a specific type of amino acid (e.g., lysine) within a peptide or portion of a peptide, may be sufficient to uniquely identify an individual peptide molecule. For example, a pattern of amino acids, such as, for example, X-X-X-Lys-X-X-X-X-Lys-X-Lys, which indicates the distribution of lysine molecules within an individual peptide molecule, where X can be any amino acid, may be searched against a known proteome of a given organism to identify the individual peptide molecule. Such information may be used to identify a macromolecule (e.g., protein) from which the peptide was derived, and may preclude the need to identify all amino acids of the peptide.

Peptide sequencing may be used to acquire information (including, for example, amino acid sequence information) from individual peptide molecules in a mixture of diverse peptide molecules. In a non-limiting example, a plurality of peptides may be immobilized on a solid surface (including, for example, a glass slide, or a glass slide whose surface has been chemically modified, a plastic slide, a multi-well plate, a cassette), amino acids from the plurality of peptides may be coupled to fluorescent reporter moieties, and the fluorescent reporter moieties may be optically detected.

Numerous commercially available optical devices can be applied in this manner. For example, conventional microscopes equipped with total internal reflection illumination and intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands or more (e.g., millions) of individual single peptides (or more) to be sequenced in a single experiment.

In an aspect, the present disclosure provides solutions to the aforementioned challenges by providing expeditious and facile methods for analyzing a polypeptide. Additionally, some aspects of the present disclosure provide compositions that facilitate effective peptide characterization and analysis. Furthermore, in some aspects the present disclosure provides kits which enable effective polypeptide analysis.

### Compounds for Edman-Like Degradation

Contrasting many uncontrolled amino acid removal methodologies, Edman degradation can provide an effective handle for controlled terminal amino acid removal. In many cases, Edman degradation comprises a terminal amino acid derivatization step, followed by a subsequent cleavage step comprising derivatized terminal amino acid removal. As each round of Edman degradation can result in the removal of a single amino acid from a subject peptide, Edman degradation is often able to facilitate step-wise, and thus numerically controlled, terminal amino acid removal.

However, Edman degradation often requires harsh conditions with poor compatibility for dyes and biological systems. For example, many forms of Edman degradation utilize trifluoroacetic acid to remove phenylisothiocyanate derivatized terminal amino acids, which can sometimes degrade peptides, nucleic acids, supramolecular biological structures (e.g., protein complexes), along with other biological species. Furthermore, Edman degradation is often slow, with each amino acid removal round often requiring at least one hour.

Responsive to the present needs for faster, chemically less intensive, and higher efficiency amino acid removal, the present disclosure provides a range of reagents, compositions, methods, and systems for terminal amino acid removal. Aspects of the present disclosure provide reagents, compositions and methods for derivatizing terminal amino acids. In many cases, such derivatized terminal amino acids comprise a degree of metastability, and can be configured to undergo chemical conversion for terminal amino acid cleavage upon catalyst, reagent, or light addition, or upon a change in conditions (e.g., pH or temperature).

In some aspects, the present disclosure provides for a composition for modifying an amine of an N-terminal amino acid of a peptide the composition comprising a reagent comprising a structure of Formula (I), or a salt, a solvate, or a derivative thereof:

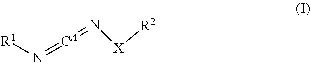

wherein:
R¹ is an electron donating group or an electron withdrawing group;
R² is a protecting group configured for cleavage from the reagent;
X is O, S, Se, or NR⁴; and
each instance of R⁴ is independently selected from the group consisting of hydrogen, optionally substituted alkyl, optionally substituted alkenyl, and optionally substituted alkynyl. The reagent may be configured to couple to the amine of the N-terminal amino acid. In some cases, the reagent is configured to couple to the amine of the N-terminal amino acid prior to cleavage of R². In some cases, the reagent is configured to not couple to the amine of the N-terminal amino acid subsequent to cleavage of R². In some cases, the reagent is configured to preferentially couple to primary amines over secondary amines. In some cases, the reagent is configured to couple to N-terminal amines of all natural proteinogenic amino acid types. In some cases, the reagent is configured to couple to N-terminal amines of all natural amino acid types (e.g., proteinogenic amino acids, post-translationally modified amino acids, chemically derivatized amino acids). In some cases, the reagent is configured to couple to N-terminal amines of synthetic amino acid types.

In some embodiments, R³ is hydrogen. In some embodiments, C^{A} of the reagent is configured to couple the N-terminal amine of the peptide prior to R² cleavage. In some embodiments, C^{A} of the reagent is configured to not couple to the N-terminal amine of the peptide subsequent to R² cleavage. In some embodiments, R² is configured for cleavage by a base. In some embodiments, the base is a halide. In some embodiments, the halide is fluoride. In some embodiments, the cleavage requires temperatures above 25° C. In some embodiments, the cleavage may be performed in neutral or alkaline organic media. In some embodiments, the cleavage may be performed in alkaline organic media.

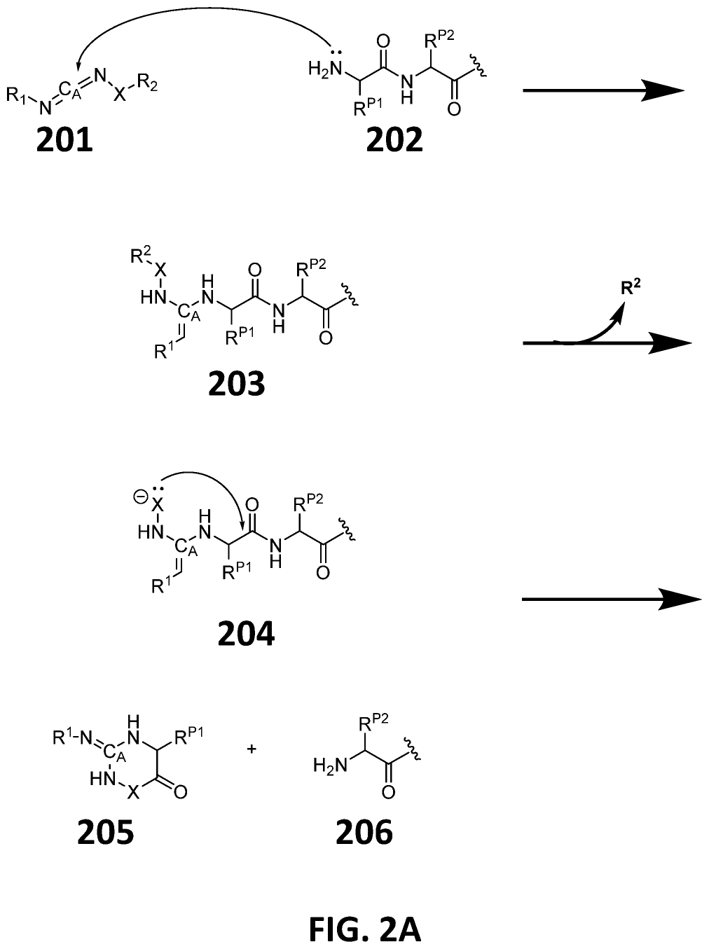

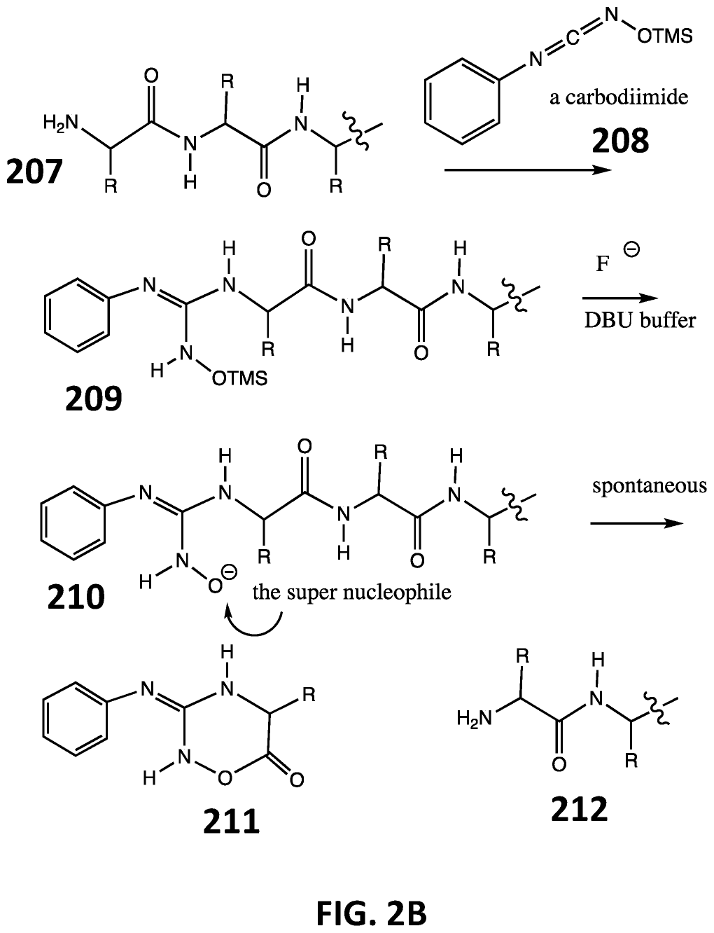

The reagent may also be configured to couple to an internal atom of the peptide. In some cases, the reagent is configured to couple to the internal amino acid after it has coupled to the terminal amino acid. In some cases, the reagent is configured to couple to the internal amino acid after R² has undergone cleavage. FIG. 2A provides a reaction scheme for N-terminal amino acid removal with a reagent comprising a structure of Formula (I). As shown in the reaction scheme of FIG. 2A, a terminal amine of a peptide 202 may couple to C^{A} of an N-terminal coupling reagent of Formula (I) 201, wherein R^{P1} and R^{P2} represent the first and second side chains of the peptide 202, counting from the N-terminus. A nitrogen adjacent to C^{A} of the coupling reagent 201 may couple to a proton prior or subsequent to the coupling between C^{A} and the peptide terminal amine, or in a concerted step including the coupling between C^{A} and the peptide terminal amine. Coupling of C^{A} with the peptide terminal amine can form intermediate 203. In many cases, this N-terminal coupling reagent is stable in the conditions required for the initial coupling step between C^{A} and the peptide terminal amine. In some cases, intermediate 203 may be in equilibrium with the starting forms of the N-terminal coupling reagent 201 and peptide 202. Intermediate 203 may cleave to form a reactive intermediate 204. The cleavage step may be chemically, catalytically, photolytically, or condition (e.g., pH) mediated. For example, intermediate 203 may be stable until contacted with a Lewis base, such as a fluoride ion, at which time it may decompose to form reactive intermediate 204. The reactive intermediate 204 may comprise reactivity for the peptide. For example, if X is O, then reactive intermediate 204 may comprise an oximate which is reactive towards a carbonyl of the peptide 202. Reaction between the reactive intermediate 204 and an internal amino acid residue of the peptide 202 may cleave the terminal amino acid (comprising R^{P1}) from the peptide to yield a cyclized product 205 comprising the terminal amino acid and at least a portion of the N-terminal coupling reagent 201, and separately yield the cleaved peptide 206.

Because the reaction requires two separate inputs for peptide cleavage, the N-terminal coupling reagent 201 and the condition, catalyst, light, or reagent for N-terminal coupling reagent cleavage, the number of terminal amino acids removed from the peptide can be controlled, such that only one terminal amino acid is removed each cycle. The cyclized product 205 may be generated in less than 2 hours following contacting the N-terminal coupling reagent 201 with the peptide 202. The cyclized product 205 may be generated in less than 1.5 hours following contacting the N-terminal coupling reagent 201 with the peptide 202. The cyclized product 205 may be generated in less than 1 hour following contacting the N-terminal coupling reagent 201 with the peptide 202. The cyclized product 205 may be generated in less than 45 minutes following contacting the N-terminal coupling reagent 201 with the peptide 202. The cyclized product 206 may be generated in less than 30 minutes following contacting the N-terminal coupling reagent 201 with the peptide 202. The cyclized product 205 may be generated in less than 20 minutes following contacting the N-terminal coupling reagent 201 with the peptide 202. The cyclized product 205 may be generated in less than 15 minutes following contacting the N-terminal coupling reagent 201 with the peptide 202. For example, in some cases, the N-terminal coupling reagent may be used to sequentially remove 10 amino acids from the N-terminus of a peptide in less than 2 and a half hours.

FIG. 2B provides an example of N-terminal amino acid removal with a carbodiimide reagent comprising a structure of Formula (I). FIG. 2B outlines a method for generating an oximate coupled to a terminal amino acid. This example provides a carbodiimide 208 configured to react with an N-terminal amine 207, and containing a trimethylsilyl (TMS) protected oxime proximal to the internal amide. In a buffer of the strong base DBU, the TMS may be cleaved to generate an oximate 209 which can rapidly couple to a carbonyl of the peptide, thereby cleaving the terminal amino acid 212 to form a cyclized product 211. Oximes (and their conjugate bases, oximates) belong to a special class of nucleophiles (referred to as “super nucleophiles”) that can benefit from what is known as the alpha-effect, in which two heteroatoms are directly bonded. Upon generation, the oximate anion may spontaneously undergo 6-exo-trig cyclization with the terminal amino acid. The conjugate acid of DBU could then potentially play the role of protonating the leaving group wherein a carbodiimide N-terminal coupling reagent couples to the terminal amine of a peptide, generating a urea complex comprising the leaving group TMS.

In some cases, the reagent is a compound of Formula (I), or a salt, a solvate, or a derivative thereof:

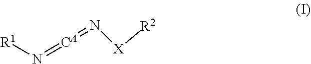

wherein:
R¹ is an electron donating group or an electron withdrawing group;
R² is a protecting group configured for cleavage from the reagent;
X is O, S, Se, or NR⁴; and
each instance of R⁴ is independently selected from the group consisting of hydrogen, optionally substituted alkyl, optionally substituted alkenyl, and optionally substituted alkynyl.

In some cases, X is O, S, or NR⁴. In some cases, X is S. In some cases, X is S or O. In some cases, X is O. In some cases, X is NR⁴. In some cases, R³ is hydrogen or an optionally substituted alkyl. In some cases, R³ is hydrogen. In some cases, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, a sulfonamide group, or any combination thereof. In certain cases, R² comprises a silyl group, for example trimethylsilyl (TMS), triethylsilyl (TES), tert-butyldimethylsilyl (TBDMS), tert-Butyldiphenylsilyl (TBDPS), triisopropylsilyl (TIPS), triisopropylsilyloxymethyl (TOM), or any combination thereof. In some cases, the silyl group comprises trimethylsilyl (TMS).

In some cases, R² is configured for cleavage by a base. In some cases, R² is configured for cleavage by an anion. In some cases, R² is configured for cleavage by a halide. In some cases, R² is configured for cleavage by fluoride. In some cases, R² is configured for cleavage by a nitrogenous buffer, such as 1,8-Diazabicyclo[5.4.0]undec-7-ene (DBU).

In some cases, R² cleavage is performed at room temperature. In some cases, R² cleavage requires a temperature of at least about 25° C., at least about 40° C., at least about 50° C., at least about 60° C., at least about 80° C., or at least about 90° C. In some cases, R² cleavage requires a temperature of about 25° C., about 40° C., about 50° C., about 60° C., about 80° C., or about 90° C. In some cases, R² cleavage requires a temperature of at most about 25° C., at most about 40° C., at most about 50° C., at most about 60° C., at most about 80° C., or at most about 90° C.

In some cases, R² is configured for cleavage in acidic organic solution (e.g., DMSO comprising octylphosphonic acid). In some cases, R² is configured for cleavage in neutral or alkaline organic solution. In some cases, R² is configured for cleavage in neutral organic solution. In some cases, R² is configured for cleavage in alkaline organic solution (e.g., piperidine in acetonitrile).

In some cases, R² cleavage generates an oxime (hereinafter used synonymously with its conjugate base, oximate), a thiooxime (hereinafter used synonymously with its conjugate base, thiooximate), an ylidene selenohydroxylamine, or an azine. In some cases, R² cleavage generates an oxime, a thiooxime, or an azine. In some cases, R² cleavage generates an oxime or a thiooximate. In some cases, R² cleavage generates an oxime. In some cases, R² cleavage generates a thiooxime. In some cases, the oxime, thiooxime, ylidene selenohydroxylamine, or azine is configured to couple to an internal atom of the peptide. In some cases, the internal amino acid is a carbonyl carbon. In some cases, the internal atom is the carbonyl carbon of the terminal amino acid to which the reagent comprising the structure of Formula (I) is coupled. In some cases, the reagent comprising the structure of Formula (I) is configured to cleave the N-terminal amino acid from the peptide upon or subsequent to coupling to the internal atom of the peptide. For example, coupling to the internal atom and N-terminal amino acid cleavage may be concerted. In some cases, cleaving the N-terminal amino acid from the peptide generates a 3-imino1,2,4-oxadiazinanone comprising at least a portion of the reagent comprising the structure of Formula (I).

In some embodiments, R¹ is an electron donating group. The electron donating group can be a group with a negative Hammett Op value. In some cases, the electron donating group is vinyl, aryl, amine, alkoxy, or alkyl, —N(CH₃)₂, —NH—NH₂, methoxy, O-alkyl, or any combination thereof. In some cases, R¹ comprises an optionally substituted alkyl group, an optionally substituted cycloalkyl group, an optionally substituted heterocycloalkyl group, an optionally substituted aryl group, or an optionally substituted heteroaryl group.

In some embodiments, R¹ is an electron withdrawing group. The electron withdrawing group can be a group with a negative Hammett Op value. The electron withdrawing group can be a group with a Hammett Op value of 0.05 to about 0.75. In some cases, the electron withdrawing group is chloro, fluoro, bromo, haloalkyl, nitroalkyl, —(C═O)—R⁴, —(C═O)—OR⁴, —(C═O)—N(R⁴)₂, perfluoroalkyl, perfluoroalkoxy, or any combination thereof.

In some cases, R⁴ is hydrogen, an optionally substituted C₁-C₉ alkyl, an optionally substituted C₁-C₉ alkenyl, or an optionally substituted C₁-C₉ alkynyl. In some cases, R⁴ is an optionally substituted C₁-C₉ alkyl. In some cases, R⁴ is hydrogen.

In some cases, R¹ comprises a detectable moiety, such as a redox active tag comprising a characteristic oxidation or reduction potential, an optically detectable moiety such as a dye, or a mass tag with a characteristic mass spectrometric signal.

In many cases, the reagent comprising the structure of Formula (I) is compatible with a wide range of solvents and conditions. For example, reagent may be soluble and stable in an organic solution (e.g., an acetonitrile and DMSO mixture or DMF). The reagent may comprise a high DMSO solubility. For example, the reagent may comprise a DMSO solubility of from about 10 mg/mL to about 1 μg/mL. The reagent may comprise a DMSO solubility of at least about 10 ng/ml, at least about 100 ng/mL, at least about 1 μg/mL, at least about 10 μg/mL, at least about 100 μg/mL, at least about 1 mg/mL, at least about 10 mg/mL, or at least about 10 mg/mL. The reagent may comprise a DMSO solubility of about 10 ng/mL, about 100 ng/mL, about 1 μg/mL, about 10 μg/mL, about 100 μg/mL, about 1 mg/mL, about 10 mg/mL, or about 10 mg/mL. The reagent may comprise a DMSO solubility of at most about 10 ng/mL, at most about 100 ng/ml, at most about 1 μg/mL, at most about 10 μg/mL, at most about 100 μg/mL, at most about 1 mg/mL, at most about 10 mg/mL, or at most about 10 mg/mL.

In some embodiments, subsequent to the cleavage, the reagent comprises an oxime or an oximate. In some embodiments, the oxime or the oximate is configured to couple to the internal amino acid of the peptide. In some embodiments, the reagent is configured to cleave the N-terminal amino acid from the peptide upon or subsequent to the coupling to the internal atom of the peptide. In some embodiments, the cleaving the N-terminal amino acid from the peptide generates a 3-imino-1,2,4-oxadiazinanone. In some embodiments, the internal atom is a carbonyl carbon of the peptide. In some embodiments, the internal atom is a carbonyl carbon of the N-terminal amino acid of the peptide.

In some embodiments, R¹ comprises an optionally substituted alkyl group, an optionally substituted aryl group, or an optionally substituted heteroaryl group. In some embodiments, R¹ comprises a detectable moiety. In some embodiments, the detectable moiety comprises a fluorescent dye, an electrochemically detectable moiety, or a mass tag.

The reagent may comprise a high degree of stability. For example, the reagent may comprise a half-life of about 1 day to 10 years when stored in dry form under dry conditions at 25° C., and in the absence of light. The reagent may comprise a half-life of at least about 1 day, at least about 10 days, at least about 30 days, at least about 100 days, at least about 1 year, at least about 2 years, at least about 5 years, or at least about 10 years when stored in dry form, in dry conditions, at 25° C., and in the absence of light. The reagent may comprise a half-life of about 1 day to 10 years when stored in DMSO at 25° C. and in the absence of light. The reagent may comprise a half-life of at least about 1 day, at least about 10 days, at least about 30 days, at least about 100 days, at least about 1 year, at least about 2 years, at least about 5 years, or at least about 10 years when stored in DMSO at 25° C. and in the absence of light. The reagent may comprise a half-life of about 1 day, about 10 days, about 30 days, about 100 days, about 1 year, about 2 years, about 5 years, or about 10 years when stored in DMSO at 25° C. and in the absence of light. The reagent may comprise a half-life of at most about 1 day, at most about 10 days, at most about 30 days, at most about 100 days, at most about 1 year, at most about 2 years, at most about 5 years, or at most about 10 years when stored in DMSO at 25° C. and in the absence of light.

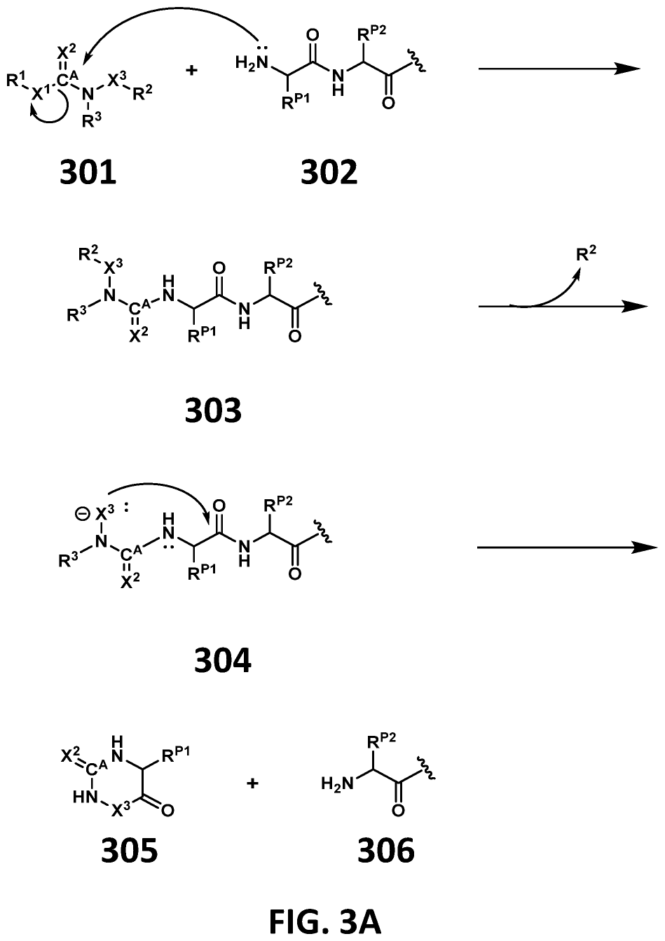

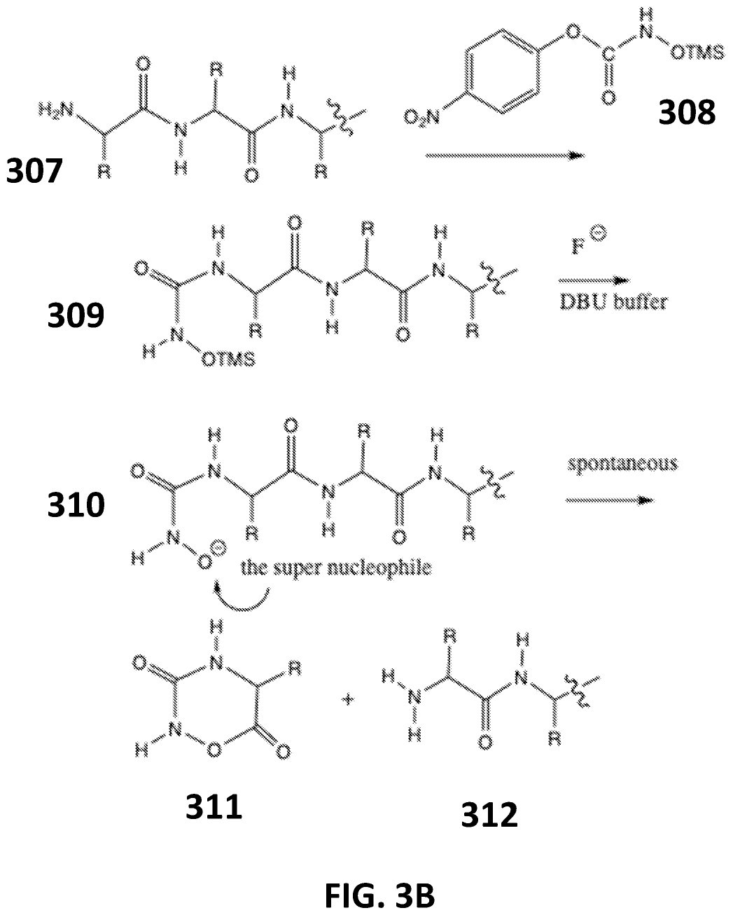

An example of a reaction scheme for N-terminal amino acid removal with a reagent comprising a structure of Formula (II) is provided in FIG. 3A. An N-terminal coupling reagent 301 may couple to an N-terminal amine of a peptide 302, of wherein R^{P1} and R^{P2} represent the first and second side chains counted from the N-terminal of the peptide. This process may comprise nucleophilic substitution of R¹—X¹ by the N-terminal amine, resulting in the formation of an intermediate 303. The intermediate may be stable in the conditions required for the initial coupling step, such that the intermediate does not react until input of light, a reagent, a catalyst, or a change in condition (e.g., a change in temperature or pH) initiates loss of R² to generate a reactive intermediate 304. The reactive intermediate may comprise an oxime, a thiooxime, an azide, or a seleno compound configured to react with a carbonyl of the peptide. The reactive intermediate may react preferentially with the carbonyl of the terminal amino acid over carbonyls of internal amino acids (in some cases, at least in part due to sterics). Coupling between the reactive intermediate 304 and the carbonyl of the terminal amino acid may liberate the terminal amino acid from the peptide, generating a cyclic product 305 comprising a portion of the N-terminal coupling reagent and the remaining portion of the peptide 306.

In some cases, the N-terminal coupling reagent 301 is activated for coupling with the N-terminal amine of the peptide 302. The N-terminal coupling reagent 301 may be provided in activated form, or may be activated in situ prior to coupling to the N-terminal amine of the peptide. For example, N-terminal coupling reagent 301 electrophilicity may be enhanced through X² alkylation or Lewis acid coupling prior to N-terminal amino acid coupling. In some cases, the N-terminal coupling reagent 301 couples to the N-terminal amine of the peptide without a prior or concurrent activating step.

The cyclic product 305 may be generated in less than about 2 hours following contact between the N-terminal coupling reagent 301 the peptide 302. The cyclic product 305 may be generated in less than about 1.5 hours following contact between the N-terminal coupling reagent 301 and the peptide 302. The cyclic product 305 may be generated in less than about 1 hour following contact between the N-terminal coupling reagent 301 and the peptide 302. The cyclic product 305 may be generated in less than about 45 minutes following contact between the N-terminal coupling reagent 301 and the peptide 302. The cyclic product 305 may be generated in less than about 40 minutes following contact between the N-terminal coupling reagent 301 and the peptide 302. The cyclic product 305 may be generated in less than about 30 minutes following contact between the N-terminal coupling reagent 301 and the peptide 302. The cyclic product 305 may be generated in less than about 20 minutes following contact between the N-terminal coupling reagent 301 and the peptide 302. The cyclic product 305 may be generated in less than 15 minutes following contact between the N-terminal coupling reagent 301 and the peptide 302.

An example of terminal amino acid removal by an N-terminal coupling reagent comprising a structure of Formula (II) is provided in FIG. 3B, in which a terminal amine of a peptide 307 nucleophilically substitutes a nitrophenolate from a carbamate N-terminal coupling reagent 308, generating a urea N-terminal derivatization with an OTMS functionalization 309. DBU buffer mediated cleavage of TMS from the N-terminal derivatized intermediate generates a reactive oximate appendage 310 which spontaneously couples to the terminal amino acid carbonyl, cleaving the terminal amino acid 311 from the peptide to form a cleaved peptide 312.

In some aspects, the present disclosure provides for a composition for modifying an amine of an N-terminal amino acid of a peptide, the composition comprising a reagent comprising a structure of Formula (II), or a salt, a solvate, or a derivative thereof:

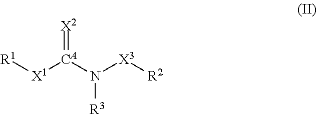

wherein:
R¹ is an electron donating group or an electron withdrawing group;
R² is a leaving group;
X¹ is O, S, SO, SR⁴, Se, SeO, SeR⁴, or NR⁴; X² is O, S, SR⁴, SOR⁴, SO₂R⁴, Se, SeR⁴, SeOR⁴, SeO₂R⁴ or NR⁴;
X³ is O, S, Se, or NR⁴; and
each instance of R³ and R⁴ is independently selected from the group consisting of hydrogen, optionally substituted alkyl, optionally substituted alkenyl, or optionally substituted alkynyl.

In some embodiments, R² is a leaving group configured for cleavage from the reagent. In some embodiments, the reagent is configured to couple to an internal atom of the peptide upon or subsequent to the cleavage. In some cases, the reagent is configured to not couple to the amine of the N-terminal amino acid subsequent to cleavage of R². In some cases, the reagent is configured to preferentially couple to primary amines over secondary amines. In some cases, the reagent is configured to couple to N-terminal amines of all natural proteinogenic amino acid types. In some cases, the reagent is configured to couple to N-terminal amines of all natural amino acid types (e.g., proteinogenic amino acids, post-translationally modified amino acids, chemically derivatized amino acids). In some cases, the reagent is configured to couple to N-terminal amines of synthetic amino acid types.

In some instances, the reagent comprising a structure of formula (II) comprises a carbamate, a carbonate, a urea, a thiocarbamate, a dithiocarbamate, a thiourea, or a guanidinium. In some cases, X¹ and X² are each independently selected from S, O and NR⁴. In some cases, X¹ and X² are each independently selected from O and NR⁴. In some cases, X¹ is NR⁴ and X² is O. In some cases, the reagent comprises a carbamate. In some cases, X³ is O. In some cases, each instance of R³ is hydrogen.

In some cases, R¹, R², or R³ comprises a detectable moiety. In some cases, R¹ or R³ comprises a detectable moiety. In some cases, the detectable moiety comprises a fluorescent dye, an electrochemically detectable moiety, or a mass tag. In some cases, the loss of X¹—R¹ from the reagent (e.g., through nucleophilic substitution at C^{A}) may be measured through a detectable moiety of R¹. For example, following reagent coupling to a population of peptides coupled to a glass slide, X¹—R¹ liberated during peptide coupling may be collected (e.g., during a wash step) and quantified based on the detectable signal from an R¹ detectable moiety. In some cases, reagent binding to a peptide may be identified through detection of an R³ detectable moiety. For example, single molecule fluorescence measurements on a population of peptides coupled to a glass slide may identify which peptides are coupled to a reagent comprising and which peptides are not coupled to a reagent through detection of R³ fluorophores. Such a method may be used to track amino acid removal from individual peptides, and maintain proper phasing over multiple amino acid removal steps.

In some cases, R¹ is an electron donating group. The electron donating group can be a group with a negative Hammett Op value. In some cases, the electron donating group is vinyl, aryl, amine, alkoxy, or alkyl, —N(CH₃)₂, —NH—NH₂, methoxy, O-alkyl, or any combination thereof. In some cases, R¹ comprises an optionally substituted alkyl group, an optionally substituted cycloalkyl group, an optionally substituted heterocycloalkyl group, an optionally substituted aryl group, or an optionally substituted heteroaryl group. In some embodiments, R¹ is amino, alkoxy, aryl, or heteroaryl, each of which is independently unsubstituted or substituted; or hydroxy. In some embodiments, R¹ is substituted phenyl. In some embodiments, R¹ is nitrophenyl. R¹ is 4-nitrophenyl.

In some cases, R¹ is an electron withdrawing group. The electron withdrawing group can be a group with a negative Hammett Op value. The electron withdrawing group can be a group with a Hammett Op value of 0.05 to about 0.75. In some cases, the electron withdrawing group is haloalkyl, nitroalkyl, —(C═O)—R⁴, —(C═O)—OR⁴, —(C═O)—N(R⁴)₂, chloro, fluoro, bromo, perfluoroalkyl, perfluoroalkoxy, or any combination thereof.

In some cases, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, a sulfonamide group, or any combination thereof. In some cases, R² comprises a silyl group. In some cases, the silyl group comprises trimethylsilyl (TMS), triethylsilyl (TES), tert-butyldimethylsilyl (TBDMS), tert-Butyldiphenylsilyl (TBDPS), triisopropylsilyl (TIPS), triisopropylsilyloxymethyl (TOM), or any combination thereof. In some cases, the silyl group comprises trimethylsilyl (TMS). In some cases, the silyl group comprises tertbutyldimethylsilyl (TBDMS).

In some cases, C^{A} is configured to couple to the N-terminal amine of the peptide. In some cases, C^{A} is configured to couple to the N-terminal amine of the peptide prior to R² cleavage (e.g., C^{A} may only be reactive towards the N-terminal amine prior to R² cleavage). In some cases, C^{A} is configured to couple to the N-terminal amine of the peptide subsequent to R² cleavage. In some cases, coupling to a N-terminal amine results in loss of R¹—X¹ from the reagent. In some cases, nucleophilic substitution at C^{A} favors a loss of R¹—X¹ over a loss of (NR³)—O—R².

In some cases, R² is configured for cleavage by a base. In some cases, the base is a halide. In some cases, the base is a fluoride. In some cases, the base is a nitrogenous lewis base. In some cases, cleavage with a base generates a modified biomolecule comprising a hydroxamic acid. In some cases, a modified biomolecule comprises a substituted hydroxamic acid. In some cases, a modified biomolecule comprises an unsubstituted hydroxamic acid. In some cases, removing an N-terminal amino acid from a biomolecule generates a 1,2,4-oxadiazinane-3,6-dione byproduct. In some cases, removing an N-terminal amino acid from a biomolecule generates a 5-substituted 1,2,4-oxadiazinane-3,6-dione byproduct. In some cases, cleavage with a base generates a modified biomolecule comprising a hydrazide. In some cases, removing an N-terminal amino acid from a biomolecule generates a 1,2,4-triazine-3,6-dione byproduct. In some cases, removing an N-terminal amino acid from a biomolecule generates a 5-substituted 1,2,4-triazine-3,6-dione byproduct.

In some cases, R² cleavage requires a temperature of at least about 25° C., at least about 40° C., at least about 50° C., at least about 60° C., at least about 80° C., or at least about 90° C. In some cases, R² cleavage requires a temperature of about 25° C., about 40° C., about 50° C., about 60° C., about 80° C., or about 90° C. In some cases, R² cleavage requires a temperature of at most about 25° C., at most about 40° C., at most about 50° C., at most about 60° C., at most about 80° C., or at most about 90° C.

In some cases, R² cleavage is heterolytic cleavage. In some cases, R² cleavage is homolytic cleavage. In some cases, R² cleavage is performed at a pH of at least about 1, at least about 2, at least about 3, at least about 4, at least about 5, at least about 6, or at least about 7. In some cases, R² cleavage is performed at a pH of about 1, about 2, about 3, about 4, about 5, about 6, or about 7. In some cases, R² cleavage is performed at a neutral or alkaline pH. In some cases, R² cleavage is performed at a pH of at most about 13, at most about 12, at most about 11, at most about 10, at most about 9, at most about 8, or at most about 7. In some cases, R² cleavage is performed at a pH of about 13, about 12, about 11, about 10, about 9, about 8, or about 7.

In some cases, loss of R² generates an oxime, a thiooxime, an ylidene selenohydroxylamine, or an azine. In some cases, R² cleavage generates an oxime, a thiooxime, or an azine. In some cases, R² cleavage generates an oxime or a thiooximate. In some cases, R² cleavage generates an oxime. In some cases, R² cleavage generates a thiooxime. In some cases, the oxime, thiooxime, ylidene selenohydroxylamine, or azine is configured to couple to an internal atom of the peptide. In some cases, the internal amino acid is a carbonyl carbon. In some cases, the internal atom is the carbonyl carbon of the terminal amino acid to which the reagent comprising the structure of Formula (II) is coupled. In some cases, the reagent comprising the structure of Formula (II) is configured to cleave the N-terminal amino acid from the peptide upon or subsequent to coupling to the internal atom of the peptide. For example, coupling to the internal atom and N-terminal amino acid cleavage may be concerted. In some cases, cleaving the N-terminal amino acid from the peptide generates a cyclic compound (e.g., a 3-imino-1,2,4-oxadiazinanone) comprising at least a portion of the reagent comprising the structure of Formula (II).

In some cases, each instance of R³ and R⁴ is independently selected from hydrogen, optionally substituted C₁-C₉ alkyl, optionally substituted C₁-C₉ alkenyl, and optionally substituted C₁-C₉ alkynyl. In some cases, at least one instance of R³ or R⁴ is an optionally substituted C₁-C₉ alkyl, an optionally substituted C₁-C₉ alkenyl, or an optionally substituted C₁-C₉ alkynyl. In some cases, R³ is hydrogen and at least one instance of R⁴ is an optionally substituted C₁-C₉ alkyl, an optionally substituted C₁-C₉ alkenyl, or an optionally substituted C₁-C₉ alkynyl. In some cases, each R⁴ is hydrogen and R³ is an optionally substituted C₁-C₉ alkyl, an optionally substituted C₁-C₉ alkenyl, or an optionally substituted C₁-C₉ alkynyl. In some cases, each R³ and R⁴ is independently hydrogen.

In many cases, the reagent comprising the structure of Formula (II) is compatible with a wide range of solvents and conditions. For example, reagent may be soluble and stable in an organic solution (e.g., a tetrahydrofuran and DMSO mixture, or DMF). The reagent may comprise a high DMSO solubility. For example, the reagent may comprise a DMSO solubility of from about 10 mg/mL to about 1 μg/mL. The reagent may comprise a DMSO solubility of at least about 10 ng/mL, at least about 100 ng/ml, at least about 1 μg/mL, at least about 10 μg/mL, at least about 100 μg/mL, at least about 1 mg/mL, at least about 10 mg/mL, or at least about 10 mg/mL. The reagent may comprise a DMSO solubility of about 10 ng/mL, about 100 ng/mL, about 1 μg/mL, about 10 μg/mL, about 100 μg/mL, about 1 mg/mL, about 10 mg/mL, or about 10 mg/mL. The reagent may comprise a DMSO solubility of at most about 10 ng/mL, at most about 100 ng/mL, at most about 1 μg/mL, at most about 10 μg/mL, at most about 100 μg/mL, at most about 1 mg/mL, at most about 10 mg/mL, or at most about 10 mg/mL.

The reagent may comprise a high degree of stability. For example, the reagent may comprise a half-life of about 1 day to 10 years when stored in dry form under dry conditions at 25° C., and in the absence of light. The reagent may comprise a half-life of at least about 1 day, at least about 10 days, at least about 30 days, at least about 100 days, at least about 1 year, at least about 2 years, at least about 5 years, or at least about 10 years when stored in dry form, in dry conditions, at 25° C., and in the absence of light. The reagent may comprise a half-life of about 1 day to 10 years when stored in DMSO at 25° C. and in the absence of light. The reagent may comprise a half-life of at least about 1 day, at least about 10 days, at least about 30 days, at least about 100 days, at least about 1 year, at least about 2 years, at least about 5 years, or at least about 10 years when stored in DMSO at 25° C. and in the absence of light. The reagent may comprise a half-life of about 1 day, about 10 days, about 30 days, about 100 days, about 1 year, about 2 years, about 5 years, or about 10 years when stored in DMSO at 25° C. and in the absence of light. The reagent may comprise a half-life of at most about 1 day, at most about 10 days, at most about 30 days, at most about 100 days, at most about 1 year, at most about 2 years, at most about 5 years, or at most about 10 years when stored in DMSO at 25° C. and in the absence of light.

Compounds herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino) phenyl, 2-(N,N-dimethylamino) phenyl, 2-(N-ethylamino) phenyl, 2-(N,N-diethylamino) phenyl, 3-aminophenyl, 3-(N-methylamino) phenyl, 3-(N,N-dimethylamino) phenyl, 3-(N-ethylamino) phenyl, 3-(N,N-diethylamino) phenyl, 4-aminophenyl, 4-(N-methylamino) phenyl, 4-(N,N-dimethylamino) phenyl, 4-(N-ethylamino) phenyl, and 4-(N,N-diethylamino) phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be at least about 1% pure, at least about 2% pure, at least about 3% pure, at least about 4% pure, at least about 5% pure, at least about 6% pure, at least about 7% pure, at least about 8% pure, at least about 9% pure, at least about 10% pure, at least about 11% pure, at least about 12% pure, at least about 13% pure, at least about 14% pure, at least about 15% pure, at least about 16% pure, at least about 17% pure, at least about 18% pure, at least about 19% pure, at least about 20% pure, at least about 21% pure, at least about 22% pure, at least about 23% pure, at least about 24% pure, at least about 25% pure, at least about 26% pure, at least about 27% pure, at least about 28% pure, at least about 29% pure, at least about 30% pure, at least about 31% pure, at least about 32% pure, at least about 33% pure, at least about 34% pure, at least about 35% pure, at least about 36% pure, at least about 37% pure, at least about 38% pure, at least about 39% pure, at least about 40% pure, at least about 41% pure, at least about 42% pure, at least about 43% pure, at least about 44% pure, at least about 45% pure, at least about 46% pure, at least about 47% pure, at least about 48% pure, at least about 49% pure, at least about 50% pure, at least about 51% pure, at least about 52% pure, at least about 53% pure, at least about 54% pure, at least about 55% pure, at least about 56% pure, at least about 57% pure, at least about 58% pure, at least about 59% pure, at least about 60% pure, at least about 61% pure, at least about 62% pure, at least about 63% pure, at least about 64% pure, at least about 65% pure, at least about 66% pure, at least about 67% pure, at least about 68% pure, at least about 69% pure, at least about 70% pure, at least about 71% pure, at least about 72% pure, at least about 73% pure, at least about 74% pure, at least about 75% pure, at least about 76% pure, at least about 77% pure, at least about 78% pure, at least about 79% pure, at least about 80% pure, at least about 81% pure, at least about 82% pure, at least about 83% pure, at least about 84% pure, at least about 85% pure, at least about 86% pure, at least about 87% pure, at least about 88% pure, at least about 89% pure, at least about 90% pure, at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Fluorosequencing

Fluorosequencing (e.g., sequencing by degradation) refers to sequencing peptides in a complex protein sample at the level of single molecules. In some embodiments, millions of individual fluorescently labeled peptides are visualized in parallel, monitoring changing patterns of fluorescence intensity as N-terminal amino acids are sequentially removed, and/or using the resulting fluorescence signatures (fluorosequences) to uniquely identify individual peptides. In some embodiments, amino acids are selectively labeled on immobilized peptides, and/or the amino acids are subjected to successive cycles of removing the peptide N-terminal residues (Edman degradation) and/or imaging the corresponding decrease of fluorescent intensity for individual peptide molecules. The methods of the present invention are capable of producing patterns sufficiently reflective of the peptide sequences to allow unique identification of a majority of proteins from a species. The resulting stair-step patterns of fluorescence decreases provide positional information of the select amino acid residues. This partial pattern is often sufficient to allow unique identification of the peptide by comparison to a reference proteome. The patterns of cleavage (even for a portion of the protein) provide sufficient information to identify a significant fraction of proteins within a known proteome, i.e. where the sequences of proteins are known in advance. In one embodiment, the single-molecule technologies of the present application allow the identification and/or absolute quantitation of a given peptide or protein in a biological sample.

In some embodiments, the methods disclosed herein can be used to perform large-scale sequencing (including but not limited to partial sequencing) of single intact peptides (not denatured) at the single molecule level by selective labeling amino acids on immobilized peptides followed by successive cycles of labeling and/or removal of the peptide amino-terminal amino acids. The methods and/or systems of the disclosure can identify amino acids in peptides, including peptides comprising unnatural amino acids. In one embodiment, the present invention comprises labeling the N-terminal amino acid with a first label and/or labeling an internal amino acid with a second label. In some embodiments, the labels are fluorescent labels. In other embodiments, the internal amino acid is Lysine. In other embodiments, amino acids in peptides are identified based on the fluorescent signature for each peptide at the single molecule level.

Various aspects of the present disclosure provide compositions and/or methods for peptide fluorosequencing, also called sequencing by degradation. A method consistent with the present disclosure may subject a peptide to fluorosequencing and/or an additional form of analysis. For example, a molecule of hemoglobin may be interrogated for glycation with immunostaining, and/or then subsequently digested and/or subjected to fluorosequencing for sequencing analysis. In one embodiment, the present invention provides a massively parallel and/or rapid method for identifying and/or quantitating individual peptide and/or protein molecules within a given complex sample.

In some embodiments, the methods of the disclosure comprise: (a) providing a polypeptide, wherein the polypeptide comprises at least one labeled internal amino acid; (b) detecting at least one signal or signal change from the polypeptide to identify at least a portion of a sequence of the polypeptide; and/or (c) subjecting the polypeptide to conditions sufficient to remove at least one amino acid from the polypeptide. In some embodiments, the at least one amino acid is removed from an N-terminus of the polypeptide. In some embodiments, subsequent to (c), the at least one labeled internal amino acid becomes a labeled terminal amino acid. In some embodiments, the at least one labeled internal amino acid is from a plurality of labeled amino acids, and/or wherein the at least one signal or signal change comprises a collective signal from the plurality of labeled amino acids. In some embodiments, the plurality of labeled amino acids comprise amino acids with different labels. In some embodiments, the different labels generate signals with different signal patterns.

In some embodiments, the at least one labeled internal amino acid comprises one or more members selected from the group consisting of lysine, glutamate, and aspartate. A peptide may comprise a label on an N-terminal amino acid, a cysteine, a lysine, a glutamic acid, an aspartic acid, a tryptophan, a tyrosine, a serine, a threonine, an arginine, a histidine, a methionine, or any combination thereof. A peptide may comprise a label on a cysteine, a lysine, a tyrosine, a histidine, a glutamate, an aspartate, a tryptophan, an arginine, a methionine, or any combination thereof. A peptide may comprise a label on a cysteine, a lysine, a tyrosine, a histidine, a glutamate, an aspartate, a tryptophan, or any combination thereof. A peptide may comprise a label on a non-canonical amino acid, such as a phosphoserine or phosphothreonine, pyroglutamic acid, hydroxyproline, azidolysine, dehydroalanine, or any combination thereof. A peptide may comprise a label on a post-translationally modified amino acid, such as a citrullinated amino acid, a methylated amino acid, a sulfurylated amino acid, a phorphorylated amino acid, a succinylated amino acid, a glycosylated amino acid, a palmitoylated amino acid, a prenylated amino acid, an acylated amino acid, an amidated amino acid, a hydroxylated amino acid, an iodinated amino acid, a chlorinated amino acid, a fluorinated amino acid, a nitrosylated amino acid, a glutathionylated amino acid, a malonated amino acid, a biotinylated amino acid, an oxidized amino acid, a reduced amino acid, or any combination thereof. Each of these amino acid residues may be labeled with a different labels. Multiple amino acid residues may be labeled with the same label such as (i) aspartic acid and glutamic acid or (ii) serine and threonine.

In some embodiments, the at least one labeled internal amino acid comprises an amino acid having a label covalently attached thereto, which label generates the at least one signal or signal change. In some embodiments, the at least one labeled internal amino acid comprises an amino acid having a dye coupled thereto, which dye generates the at least one signal or signal change. In some embodiments, the at least one signal or signal change is an optical signal. In some embodiments, the at least one signal or signal change is detected with an optical detector having single-molecule sensitivity. In some embodiments, the at least one signal or signal change comprises a plurality of signals of different intensities. In some embodiments, the at least one signal or signal change comprises a plurality of signals of different frequencies or frequency ranges.

In some embodiments, the label is coupled to an internal monomeric subunit of the plurality of monomeric subunits. In some embodiments, the label is an amino acid specific label. In some embodiments, the amino acid specific label comprises a methionine specific label, an arginine specific label, a histidine specific label, a tyrosine specific label, a carboxylic acid R-group specific label, a lysine specific label, a cysteine specific label, a tryptophan specific label, or any combination thereof. In some embodiments, the amino acid specific label comprises a non-natural amino acid specific label. In some embodiments, the non-natural amino acid specific label is a phosphoserine specific label, phosphothreonine specific label, pyroglutamic acid specific label, hydroxyproline specific label, azidolysine specific label, or dehydroalanine specific label. In some embodiments, the label is a fluorescent label. In some embodiments, the label is a dye.

In some embodiments, the at least one amino acid is removed from the polypeptide by a degradation reaction. In some embodiments, the method further comprises processing at least the portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and/or (ii) using the polypeptide identified in (i) to quantify the polypeptide or a protein from which the polypeptide was derived. In some embodiments, in (a), less than all amino acids of the polypeptide are labeled. In some embodiments, the method further comprises (i) repeating (b) and/or (c) to detect at least one additional signal or signal change from the polypeptide and/or (ii) using the at least one signal or signal change and/or the at least one additional signal or signal change to identify the at least the portion of the sequence.

A characteristic feature of many fluorosequencing methods is coupling amino acid labels to a peptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject peptide or protein. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as (i) aspartic acid and/or glutamic acid or (ii) serine and/or threonine.

In one embodiment, a method of labeling a peptide comprises: a) providing, i) a peptide having at least one Cysteine amino acid, at least one Lysine amino acid, an N-terminal end, an amino acid having at least one carboxylate side group, a C-terminal end, and/or at least one Tryptophan amino acid, and/or ii) a first compound, iii) a second compound, iv) a third compound, v) a fourth compound, and/or vi) a fifth compound; and/or b) labeling the Cysteine with the first compound, c) labeling the Lysine with the second compound, d) labeling the N-terminal end with the third compound, e) labeling the carboxylate side group and/or the C-terminal end with the fourth compound; and/or f) labeling the Tryptophan with the fifth compound for providing a peptide having specific labels. In one embodiment, steps b-f are sequential in order from b-f. In one embodiment, the labeling in steps b-f is performed in one (a single) solution. In one embodiment, steps b-f are sequential in order from b-f and/or performed in one solution. In one embodiment, the first compound is iodoacetamide. In one embodiment, the second compound is 2-methylthio-2-imadazoline hydroiodide (MDI). In one embodiment, the third compound is 1-(4,4-dimethyl-2,6-dioxocyclohexylidene)-3-methylbutyl diethyl phosphate (Phos-ivDde). In one embodiment, the fourth compound is selected from the group consisting of benzylamine (BA), 3-dimethylaminopropylamine, and isobutylamine. In one embodiment, the fifth compound is 2,4-dinitrobenzenesulfenyl chloride.

In one embodiment, disclosed herein is a method of treating a peptide comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label; b) treating the plurality of immobilized peptides under conditions such that each N-terminal amino acid of each peptide is removed; and/or c) detecting the first signal for each peptide at the single molecule level. In one embodiment, the second label is attached via an amine-reactive dye. In one embodiment, the second label is selected from the group consisting of fluorescein isothiocyanate, rhodamine isothiocyanate or other synthesized fluorescent isothiocyanate derivative. In one embodiment, portions of the emission spectrum of the first label do not overlap with the emission spectrum of the second label. In one embodiment, the removal of the N-terminal amino acid in step b) is done under conditions such that the remaining peptides each have a new N-terminal amino acid. In one embodiment, the method further comprises the step d) adding the second label to the new N-terminal amino acids of the remaining peptides. In one embodiment, among the remaining peptides the new end terminal amino acid is Lysine. In one embodiment, the method further comprises the step e) detecting the next signal for each peptide at the single molecule level.

In one embodiment, the method further comprises a step of treating the immobilized peptides under conditions such that each N-terminal amino acid of each peptide is removed by a sufficient condition; and/or a step of detecting the signal for each peptide at the single molecule level. In one embodiment, the label is attached to a fluorophore by a covalent bond. In one embodiment, the fluorophore and/or the covalent bond is resistant to degradation effects. In some embodiments, the fluorophore is a fluorophore that remains intact and/or attached to the label during sequencing by degradation.

The repetitive detection of signal for each peptide at the single molecule level results in a pattern. The resulting pattern is unique to a single-peptide within the plurality of immobilized peptides. In one embodiment, the single-peptide pattern is compared to the proteome of an organism to identify the peptide, one embodiment, the intensity of the labels are measured amongst the plurality of immobilized peptides. In some embodiments, the peptides are immobilized via Cysteine residues. In some embodiments, the detecting in step c) is done with optics capable of single-molecule resolution. In a specific embodiment, one or more of the plurality of peptides comprises one or more unnatural amino acids.

In some embodiments, the emission spectrum of the first label do not overlap with the emission spectrum of the second label. In some embodiments, the removal of the N-terminal amino acid in step b) is done under conditions such that the remaining peptides each have a new N-terminal amino acid. In one embodiment, the method further comprises the step d) adding the second label to the new N-terminal amino acids of the remaining peptides. In some embodiments, among the remaining peptides, the new end terminal amino acid is Lysine. In one embodiment, the method further comprises the step e) detecting the next signal for each peptide at the single molecule level. In one embodiment, the intensity of the first and/or second labels are measured amongst the plurality of immobilized peptides. In some embodiments, the peptides are immobilized via Cysteine residues. In some embodiments, the detecting in step c) is done with optics capable of single-molecule resolution. In one embodiment, one or more of the plurality of peptides comprises one or more unnatural amino acids. In one embodiment, the unnatural amino acids comprises moieties selected from the group consisting of hydroxycarboxylates, aldehydes, thiols, and olefins. In one embodiment, one or more of the plurality of peptides comprises one or more beta amino acids.

In one embodiment, the method further comprises a step of treating an immobilized peptide (e.g., a support or bead) under conditions such that each N-terminal amino acid of each peptide is removed by degradation reaction using reagents of the disclosure; and/or a step of detecting the signal for each peptide at the single molecule level. In some embodiments, the N-terminal amino acid removing step and/or the detecting step are successively repeated from about 1 time to about 5 times, from about 5 times to about 10 times, from about 10 times to about 20 times, from about 20 times to about 30 times, from about 30 times to about 40 times, from about 40 times to about 50 times, from about 50 times to about 60 times, from about 60 times to about 70 times, from about 70 times to about 80 times, from about 80 times to about 90 times, or from about 90 times to about 100 times. In some embodiments, the N-terminal amino acid removing step and/or the detecting step are successively repeated at least about 5 times, at least about 10 times, at least about 20 times, at least about 30 times, at least about 40 times, at least about 50 times, at least about 60 times, at least about 70 times, at least about 80 times, at least about 90 times, or at least about 100 times. In some embodiments, the N-terminal amino acid removing step and/or the detecting step are successively repeated about 5 times, about 10 times, about 20 times, about 30 times, about 40 times, about 50 times, about 60 times, about 70 times, about 80 times, about 90 times, or about 100 times. In some embodiments, the N-terminal amino acid removing step and/or the detecting step are successively repeated at most about 5 times, at most about 10 times, at most about 20 times, at most about 30 times, at most about 40 times, at most about 50 times, at most about 60 times, at most about 70 times, at most about 80 times, at most about 90 times, or at most about 100 times.

A label may comprise a detectable moiety. The detectable moiety (i.e., label) may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different detectable moiety that uniquely identifies the label by its type.

Labeling specificity can be a major challenge for a fluorosequencing method. In many cases, a label may comprise reactivity toward a plurality of amino acid types. For example, some maleimide labels can react with cysteine, lysine, and/or N-terminal amines. Discriminating between similarly reactive amino acid residues can require precise ordering of labeling steps. In the above maleimide example, lysine may be discriminated from cysteine by first reacting cysteine with a cysteine specific labeling step (e.g., iodoacetamide coupling at pH 7-8), thereby preventing further cysteine labeling in a subsequent lysine labeling step. A method may comprise cysteine labeling prior to lysine labeling. A method may comprise cysteine labeling prior to aspartate and/or glutamate labeling. A method may comprise cysteine labeling prior to tryptophan labeling. A method may comprise cysteine labeling prior to tyrosine labeling. A method may comprise cysteine labeling prior to serine and/or threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise tyrosine labeling prior to lysine labeling. A method may comprise lysine labeling prior to serine and/or threonine labeling. A method may comprise lysine labeling prior to arginine labeling. A method may comprise carboxylate side chain (e.g., glutamate and/or aspartate side chain) labeling prior to tryptophan labeling. A method may comprise carboxylate side chain (e.g., glutamate and/or aspartate side chain) labeling prior to tyrosine labeling. A method may comprise carboxylate side chain (e.g., glutamate and/or aspartate side chain) labeling prior to serine labeling. A method may comprise carboxylate side chain (e.g., glutamate and/or aspartate side chain) labeling prior to serine and/or threonine labeling. A method may comprise carboxylate side chain (e.g., glutamate and/or aspartate side chain) labeling prior to histidine labeling. A method may comprise carboxylate side chain (e.g., glutamate and/or aspartate side chain) labeling prior to arginine labeling. A method may comprise C-terminal carboxylate labeling prior to lysine labeling. A method may comprise C-terminal carboxylate labeling prior to tyrosine labeling. A method may comprise C-terminal carboxylate labeling prior to histidine labeling. A method may comprise C-terminal carboxylate labeling prior to tryptophan labeling. A method may comprise C-terminal carboxylate labeling prior to glutamate and/or aspartate labeling. A method may comprise C-terminal carboxylate labeling prior to serine and/or threonine labeling. A method may comprise at least about 2, at least about 3, at least about 4, at least about 5, or at least about 6 amino acid labeling steps performed in a sequence configured to minimize or prevent label cross-reactivity (e.g., labeling more than the intended type or types of amino acids). A method may comprise 2, 3, 4, 5, or 6 amino acid labeling steps performed in a sequence configured to minimize or prevent label cross-reactivity (e.g., labeling more than the intended type or types of amino acids).

A label may reversibly or irreversibly bind to an amino acid type, and thus may be chemically (e.g., by addition of a cleavage reagent) or physically (e.g., by addition of heat or light) decoupled from a target peptide. A method may thus comprise blocking a first amino acid, labeling a second amino acid type (e.g., threonine), unblocking the first amino acid type, and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, guanidinium-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

Labeling specificity can be a major challenge for a fluorosequencing method. In many cases, a label may comprise reactivity toward a plurality of amino acid types. For example, some maleimide labels can react with cysteine, lysine, and N-terminal amines. A number of strategies may be employed to utilize or prevent such cross-reactivity. A method may comprise sequential amino acid labeling, for example to ensure that a multi-specific label is added to a system after one or more amino acid types with which the multi-specific label is configured to couple are chemically blocked or labeled, and therefore unable to react with the multi-specific label.

Fluorosequencing may comprise removing peptides through techniques such as degradation by reaction with a reagent of the disclosure following or preceding subject peptide detection. Sequential peptide removal may generate sequence or position-specific information. For example, a reduction in fluorescence following an N-terminal amino acid removal step may indicate that a labeled amino acid, and/or thus that a specific type of amino acid, was disposed at a peptide N-terminal. Removal of each amino acid residue can be carried out with a variety of different techniques including a reaction with a reagent of the disclosure.

In one embodiment, the label is attached to a fluorophore by a covalent bond. In one embodiment, the fluorophore and/or the covalent bond is resistant to degradation effects of the degradation reactions disclosed herein. A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and/or (5)6-napthofluorescein. In some embodiments, a labeling moiety is tetramethylrhodamine, Si-Rhodamine, Rhodamine B, Rhodamine B N, N′-dimethylethylenediamine, Rhodamine B sulfenyl chloride, Alexafluor555, Alexa Fluor 405, Atto647N, (5)6-napthofluorescein, variants and/or derivations thereof, etc. In one embodiment, the fluorophore is Atto647N. In one embodiment, the fluorophore is Atto643. In one embodiment, the fluorophore is Alexa555. In one embodiment, the fluorophore is Atto495. In one embodiment, the fluorophore is selected from the group consisting of tetramethylrhodamine, Si-Rhodamine, Rhodamine B, Rhodamine B N, N′-dimethyl ethylenediamine, Rhodamine B sulfenyl chloride, Alexafluor555, Alexa Fluor 405, Atto647N, (5)6-napthofluorescein, variants and/or derivations thereof. The labeling moiety may be a fluorescent peptide or protein or a quantum dot. In some embodiments, the labeling moiety is a quantum dot. In some embodiments, two-color single molecule peptide sequencing reactions can be used to identify and/or quantify biomolecules by using two or more fluorescent molecules. In some embodiments, a labeling moiety can comprise a cyanine dye, diazo dye, organoboron dye, or a boron-dipyrromethane (BODIPY) dye.

In some embodiments, amino acids can be removed from the carboxy terminus of a biomolecule, revealing C-terminal sequences instead of N-terminal sequences. In some embodiments, an engineered carboxypeptidase is used to mimic N-terminal degradation. In some embodiments, the chemical cleavage comprises cyanogen bromide cleavage, BNPS-skatole cleavage, formic acid cleavage, hydroxylamine cleavage, 2-nitro-5-thiocyanobenzoic acid cleavage, or any combination thereof.

In some embodiments, the methods disclosed herein comprise identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label, wherein a subset of the plurality of peptides comprise an N-terminal Lysine having both the first and/or second label; b) treating the plurality of immobilized peptides under conditions such that each N-terminal amino acid of each peptide is removed by a degradation reaction of the disclosure; and/or c) detecting the first signal for each peptide at the single molecule level under conditions such that the subset of peptides comprising an N-terminal Lysine is identified. It is preferred that the removal of the N-terminal amino acid in step b) is done under conditions such that the remaining peptides each have a new N-terminal amino acid. The present invention further contemplates in one embodiment, a method of identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label, wherein a subset of the plurality of peptides comprise an N-terminal acid that is not Lysine; b) treating the plurality of immobilized peptides under conditions such that each N-terminal amino acid of each peptide is removed by an Edman degradation reaction; and/or c) detecting the first signal for each peptide at the single molecule level under conditions such that the subset of peptides comprising an N-terminal amino acid that is not Lysine is identified. It is preferred that the removal of the N-terminal amino acid in step b) is done under conditions such that the remaining peptides each have a new N-terminal amino acid. It is preferred that the peptides are immobilized via Cysteine residues. In one embodiment, one or more of the plurality of peptides comprises one or more unnatural amino acids. In one embodiment, the unnatural amino acids comprise moieties selected from the group consisting of hydroxycarboxylates, aldehydes, thiols, and/or olefins, one embodiment, one or more of the plurality of peptides comprises one or more beta amino acids.

Detecting the immobilized peptide may comprise capturing an image comprising the peptide. The image may comprise a spatial address specific to the peptide. A plurality of peptides may be detected in a single image, wherein one or more of the peptides may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and/or metal surfaces (Gold coating)), coating schemes (spin-coating, dip-coating, electron beam deposition for metals, thermal vapor deposition and/or plasma enhanced chemical vapor deposition) and/or functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and/or modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labeling moieties (e.g., amino acid labels) coupled to the peptide. The sequencing technique may comprise imaging a plurality of peptides or proteins to determine the presence of one or more labeling moieties on individual peptides from among the plurality of peptides. The sequencing technique may comprise imaging from about 10³ to about 10⁴, from about 10⁴ to about 10⁵, from about 10⁵ to about 10⁶, from about 10⁶ to about 10⁷, or from about 10⁷ to about 10⁸ proteins or peptides. The sequencing technique may comprise imaging at least about 10³, at least about 10⁴, at least about 10⁵, at least about 10⁶, at least about 10⁷, or at least about 10⁸ or more proteins or peptides (e.g., imaging a portion of a surface comprising at least about 10³ to at least about 10⁸ proteins or peptides). The sequencing technique may comprise imaging about 10³, about 10⁴, about 10⁵, about 10⁶, about 10⁷, or about 10⁸ or more proteins or peptides (e.g., imaging a portion of a surface comprising about 10³ to about 10⁸ proteins or peptides). The sequencing technique may comprise imaging at most about 10³, at most about 10⁴, at most about 10⁵, at most about 10⁶, at most about 10⁷, or at most about 10⁸ or more proteins or peptides (e.g., imaging a portion of a surface comprising at most about 10³ to at most about 10⁸ proteins or peptides).

These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 11) (wherein ‘K’ denotes lysine, ‘D’ denotes aspartate, ‘Y’ denotes tyrosine, ‘A’ denotes alanine, and ‘G’ denotes glycine), and/or may comprise labels coupled to each lysine and/or tyrosine residue. A first image comprising the C-terminal immobilized peptide may indicate the presence of two lysines and/or one tyrosine in the peptide. The N-terminal amino acid may be removed (e.g., by Edman degradation), such that a second image comprising the C-terminal immobilized peptide may indicate the presence of one lysine and/or one tyrosine in the peptide. This process may be repeated until a sequence of KXXXXXXXXXXKX is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and/or comparing these locations to known peptide sequences, which may identify the entire list of amino acid residues in the peptide sequence. For example, identifying the positions of the lysines and/or cysteines in a 40 amino acid fragment of a human protein may uniquely identify the protein (e.g., only one human protein contains the specific pattern of lysine and/or cysteine residues identified in the 40 amino acid fragment).

An imaging method may involve a variety of different spectrophotometric and/or microscopy methods, such as fluorimetry, diffuse reflectance, interferometric scattering, Raman, resonance enhanced Raman, infrared absorbance, visible light absorbance, ultraviolet absorbance, and/or fluorescence. In some embodiments, a conventional microscope equipped with total internal reflection illumination and/or an intensified charge-couple device (CCD) detector may be used for imaging. Depending on the absorption and/or emission spectra of fluorescent labels employed, appropriate filters can be used to record the emission intensity of the labels. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and/or imaging a subject peptide, the position of the labeled amino acid residue can be determined in the peptide.

For each cycle, the fluorescence intensity of a label is recorded after each cleavage step. The loss and/or uptake of a label after each cleavage step and/or coupling step serves as a 1) counter for the number of amino acid residues removed, and/or 2) an internal error control indicating the successful completion of each round of degradation for each immobilized peptide.

Following image processing to filter noise and/or identify the location of peptides, and/or to map the locations of the same peptides across the set of collected images, intensity profiles for labels are associated with each peptide as a function of a cycle. The label intensity profile of each error free peptide sequencing reaction is transformed into a binary sequence in which a “1” precedes a drop in fluorescence intensity and/or its location (i.e., position within the binary sequence). Identifies the number of cycles performed. A database of predicted potential proteins is used as a reference database. The binary intensity profile of each peptide, as generated from the single molecule microscopy, is then compared to the entries in the simulated peptide database. Quantification can be accomplished by counting peptides derived from each protein observed.

The present disclosure provides a range of methods for cleaving a terminal amino acid from a peptide with high efficiency and specificity. Amino acid residue can be carried out with a variety of different techniques, including terminal amino acid removal with a reagent comprising a structure of Formula (I) or (II). The technique may include using degradation to remove the terminal amino acid residue. Alternatively, the techniques may involve using an enzyme to remove the terminal amino acid residue. For example, a terminal amino acid may be modified with a reagent comprising a structure of Formula (I) or (II), and then cleaved by an enzyme which recognizes terminal amino acids derivatized with such a reagent. These terminal amino acid residues may be removed from either the C-terminus or the N-terminus of the peptide chain.

For example, a method of the present disclosure may comprise coupling a detectable label to an amino acid of the peptide, wherein the detectable label comprises a specificity for the side chain of the amino acid; detecting a signal from the detectable label coupled to the peptide; coupling an N-terminal coupling reagent to the N-terminal amino acid of the peptide; and cleaving the N-terminal coupling reagent, thereby activating the N-terminal coupling agent to remove at least one amino acid from the peptide. The peptide may be immobilized to a support, such as a glass slide. The peptide may be immobilizing subsequent to coupling the detectable label to the amino acid of the peptide, and prior to detecting the signal from the detectable label. The peptide may be immobilized to the support by a C-terminus. The peptide may be immobilized to the support by an N-terminus. The peptide may be immobilized to the support by a cysteine thiol. The peptide may be immobilized to the support by coupling (e.g., non-covalently binding) to a peptide (e.g., an antibody, a T-cell receptor, a pore protein, a catalytically inactive protease, or any combination thereof) coupled to the support. The detectable label may comprise at least one type of detectable label, at least two types of detectable labels, at least three types of detectable labels, at least four types of detectable labels, at least five types of detectable labels, or at least six types of detectable labels. The N-terminal coupling reagent may comprise a compound comprising a structure of Formula (I). The N-terminal coupling reagent may comprise a compound comprising a structure of Formula (II). The N-terminal coupling reagent may comprise a carbodiimide or a urea moiety. In some cases, cleavage of the N-terminal coupling reagent generates an oxime or an oximate. In some cases, removing at least one amino acid from the peptide comprises coupling the oxime or the oximate and a carbonyl of the peptide. In some cases, coupling the N-terminal coupling reagent to the N-terminal amino acid of the peptide and removing the at least one amino acid from the peptide are performed in less than 2 hours, less than 1.5 hours, less than 1 hour, less than 45 minutes, less than 30 minutes, less than 20 minutes, or less than 15 minutes. In some cases, the method comprises repeating detection and amino acid removal steps at least once. In some cases, the method comprises identifying an unlabeled amino acid of the peptide. In some cases, the method comprises identifying a sequence of the peptide. In some cases, the method comprises detecting a signal from the N-terminal coupling reagent subsequent to coupling the N-terminal coupling reagent to the N-terminal amino acid of the peptide. In some cases, the at least one amino acid removed from the peptide comprises the N-terminal amino acid.

A label, reporter moiety, or protecting group of the present disclosure may be configured to withstand conditions for removing one or more of amino acid residues from a peptide. Some non-limiting examples of potential reporter moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-napthofluorescein. A reporter moiety may comprise fluorescent peptide (e.g., green fluorescent protein or a variant thereof) or an optically detectable material, such as a carbon nanotube, a nanorod, or a quantum dot.

Peptide detection or imaging may comprise immobilizing the peptide on a surface. The peptide may be immobilized to the surface by coupling a peptide-derived cysteine residue, the peptide N-terminus, or the peptide C-terminus with the surface or with a reagent coupled to the surface. The peptide may be immobilized by reacting the cysteine residue with the surface or with a capture reagent coupled to the surface. The peptide may be immobilized by coupling the peptide C-terminus or N-terminus with a capture moiety described herein. The peptide may be immobilized on a surface.

In some cases, disclosed herein is a method for analyzing a biomolecule comprising: (a) coupling a detectable label to an amino acid of the biomolecule, wherein the detectable label comprises a specificity for a side chain of the amino acid; (b) detecting a signal from the detectable label coupled to the biomolecule; (c) coupling an N-terminal coupling reagent to an N-terminal amino acid of the biomolecule to form a modified biomolecule, wherein the modified biomolecule comprises an oxime group; and (d) cleaving the modified biomolecule, wherein the cleaving removes at least one amino acid from the biomolecule to form a cleaved biomolecule. In some cases, the detectable label is a dye. In some cases, the dye is a cyanine dye, diazo dye, organoboron dye, or a combination thereof. In some cases, the dye is a boron-dipyrromethane (BODIPY) dye. In some cases, the detectable label is a fluorescent label. In some cases, the biomolecule is a polypeptide. In some cases, the biomolecule is a protein.

In some cases, the detectable label generates at least one signal or at least one signal change. In some cases, the at least one signal or the at least one signal change is an optical signal. In some cases, the at least one signal or the at least one signal change comprises a plurality of signals of different intensities. In some cases, the at least one signal or the at least one signal change comprises a plurality of signals of different frequencies or signals of different frequency ranges. In some cases, the at least one signal or signal change is detected with an optical detector having single-molecule sensitivity. In some cases, the detecting comprises fluorimetry. In some cases, the detecting comprises imaging. In some cases, the detecting identifies a sequence of the biomolecule.

In some cases, the detectable label is coupled to an internal amino acid of the biomolecule. In some cases, the internal amino acid to which the detectable label couples is selected from the group consisting of cysteine, lysine, tyrosine, histidine, glutamate, aspartate, tryptophan, arginine, serine, threonine, and methionine. In some cases, the detectable label is an amino acid specific label. In some cases, the detectable label comprises a methionine specific label, an arginine specific label, a histidine specific label, a tyrosine specific label, a carboxylic acid R-group specific label, a lysine specific label, a cysteine specific label, a tryptophan specific label, or any combination thereof. In some cases, the detectable label comprises at least two types of detectable labels, each of which couples to a different type of amino acid selected from the group consisting of cysteine, lysine, tyrosine, histidine, glutamate, aspartate, tryptophan, arginine, serine, threonine, and methionine.

In some cases, the detectable label comprises at least three types of detectable labels, each of which couples to a different type of amino acid selected from the group consisting of cysteine, lysine, tyrosine, histidine, glutamate, aspartate, tryptophan, arginine, serine, threonine, and methionine. In some cases, the detectable label comprises at least four types of detectable labels, each of which couples to a different type of amino acid selected from the group consisting of cysteine, lysine, tyrosine, histidine, glutamate, aspartate, tryptophan, arginine, serine, threonine, and methionine. In some cases, the detectable label comprises at least five types of detectable labels, each of which couples to a different type of amino acid selected from the group consisting of cysteine, lysine, tyrosine, histidine, glutamate, aspartate, tryptophan, arginine, serine, threonine, and methionine.

In some cases, the amino acid specific label comprises a non-natural amino acid specific label. In some cases, the non-natural amino acid specific label is a phosphoserine specific label, phosphothreonine specific label, pyroglutamic acid specific label, hydroxyproline specific label, azidolysine specific label, or dehydroalanine specific label. In some cases, the amino acid to which the detectable label couples is a post-translationally modified amino acid. In some cases, the post-translationally modified amino acid is citrullinated, methylated, sulfurylated, phorphorylated, succinylated, glycosylated, palmitoylated, prenylated, acylated, amidated, hydroxylated, iodinated, chlorinated, fluorinated, nitrosylated, glutathionylated, malonated, biotinylated, oxidized, reduced, or any combination thereof.

In some cases, the N-terminal coupling reagent comprises a carbamate group. In some cases, the N-terminal coupling reagent is a compound with a structure of Formula (II), or a salt, a solvate, or a derivative thereof:

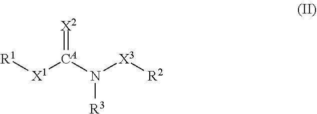

wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen, wherein the reagent modifies the N-terminal amino acid of the peptide.

In some cases, R¹ is an electron withdrawing group. In some cases, R¹ is an electron donating group. In some cases, R¹ is amino, alkoxy, aryl, or heteroaryl, each of which is independently unsubstituted or substituted; or hydroxy. In some cases, R¹ is substituted phenyl. In some cases, R¹ is nitrophenyl.

In some cases, R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group. In some cases, R² comprises a silyl group. In some cases, the silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM). In some cases, R² is tert-butyldimethylsilyl. In some cases, R² is trimethylsilyl.

In some cases, X¹ is O. In some cases, X² is O. In some cases, X³ is O.

In some cases, each R³ and R⁴ is independently C₁-C₉ alkyl, C₁-C₉ alkenyl, or C₁-C₉ alkynyl, each of which is independently unsubstituted or substituted. In some cases, R³ is hydrogen. In some cases, R⁴ is hydrogen.

In some cases, the reagent has the structure:

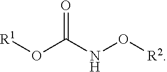

In some cases, disclosed herein is a method comprising: (a) providing a polypeptide immobilized to a support, wherein the polypeptide comprises at least one labeled internal amino acid; (b) detecting at least one signal or signal change from the polypeptide immobilized to the support to identify at least a portion of a sequence of the polypeptide; and (c) subjecting the polypeptide to conditions sufficient to remove at least one amino acid from the polypeptide to form a cleaved polypeptide via a hydroxamic acid or a hydrazide intermediate.

### Selective Amino Acid Labeling

Various aspects of the present disclosure provide methods for selectively labeling types (e.g., lysine, tyrosine, or phosphotyrosine) or groups (e.g., carboxylate side chain-containing or aromatic side chain-containing) of amino acids. A composition, system, or method of the present disclosure may selectively label cysteine, lysine, tyrosine, histidine, glutamic acid, aspartic acid, tyrosine, threonine, serine, arginine, N-terminal amines, C-terminal carboxyl-groups, or any combination thereof. A composition, system, or method may selectively label a group of amino acids, for example, a substituted maleimide reagent may couple to lysine and cysteine residues present in a sample.

The present disclosure provides a range of reagents for selectively labeling specific amino acid types (e.g., cysteine) and groups of amino acids (e.g., carboxylate side chain-containing amino acids, such as glutamate and aspartate). Non-limiting examples of cysteine-specific labels may include certain iodoacetamides, thiols, benzyl and allyl halides, selenocyanates, maleimides, and alkynes (e.g., certain alkynoic amides). In some cases, a maleimide may be configured to couple to cysteine and lysine. An example of a cysteine labeling scheme, in which a cysteine thiol nucleophilically couples to an iodoacetamide, is outlined in Scheme 1 below.

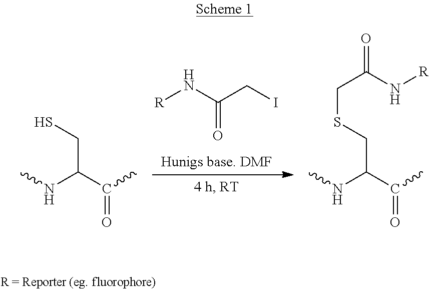

Non-limiting examples of lysine-specific labels may include certain thiocyanates and isothiocyanates, maleimides, aldehydes, isatoic anhydrides, and NHS esters. For example, a lysyl butylamine sidechain may be selectively coupled to an NHS ester, as outlined in Scheme 2.

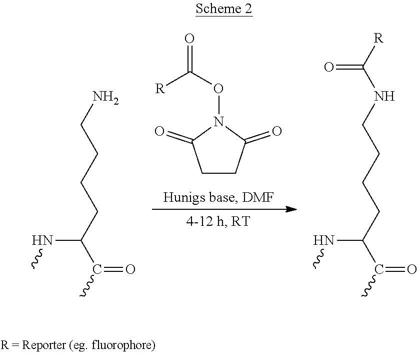

Peptide carboxylates (e.g., glutamate, aspartate, and C-terminal carboxylates) may be labeled through nucleophilic coupling steps. An example of such a coupling process is provided in Scheme 3, which illustrates carboxyl conversion to amide conversion via amine-based nucleophilic substitution.

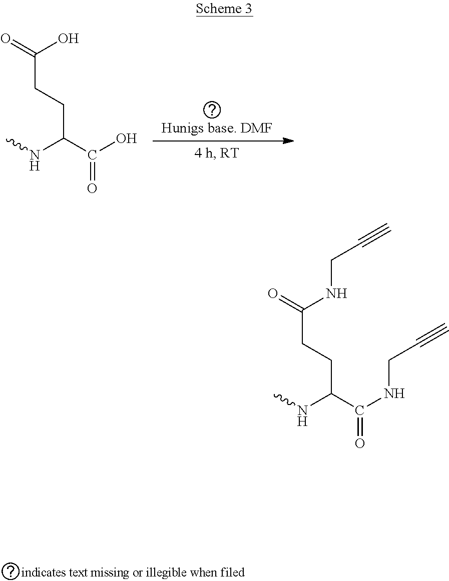

Scheme 4 provides an example of tyrosine-specific labeling. The position adjacent (e.g. ortho to) the tyrosine phenol hydroxyl carbon can be labeled through a two-step labeling process using a bifunctional diazonium reagent. Following diazo-coupling to tyrosine, a second reagent (such as a dithiolane) may optionally be coupled to the diazo label (e.g., to selectively couple a detectable moiety to the labeled tyrosine). Alternatively, the diazonium reagent may comprise a detectable moiety or may lack chemically reactive handles for further coupling.

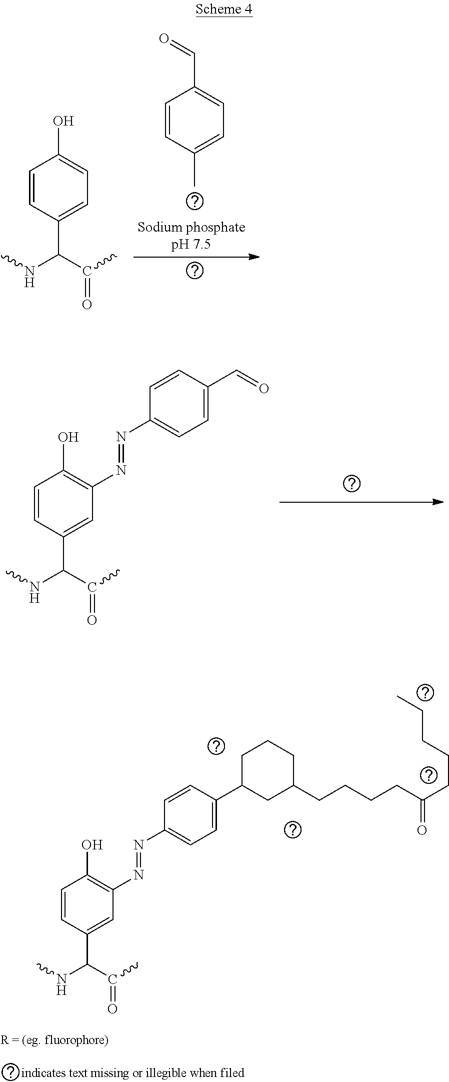

Scheme 5 provides an example of a histidine coupling scheme. A histidine imidazole nitrogen can be labeled through a two-step labeling process using an alpha-beta unsaturated carbonyl compound, such as 2-cyclohexenone. The alpha-beta unsaturated carbonyl compound may react with histidine in a nucleophilic addition reaction. The alpha-beta unsaturated carbonyl may comprise a detectable moiety. Following histidine coupling, the alpha-beta unsaturated carbonyl may be further coupled to an additional label, such as a dithiolane. Histidine may alternatively be selectively coupled to an epoxide reagent.

### Scheme 5

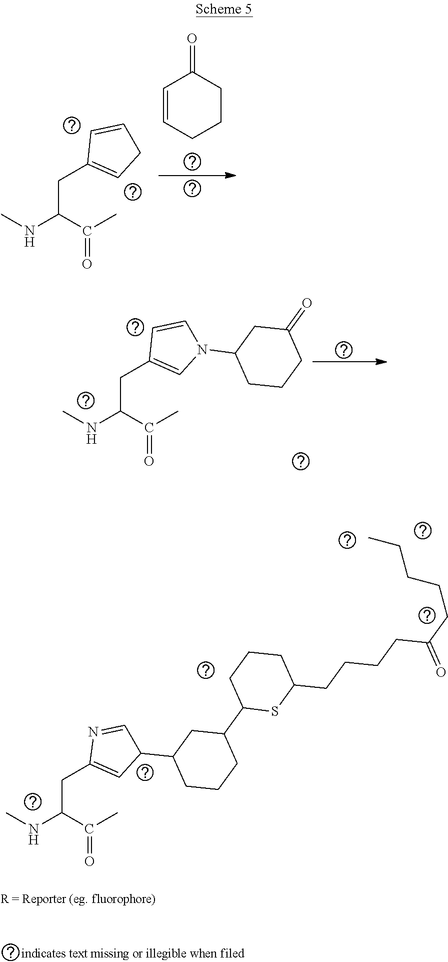

Scheme 6 provides an example of an arginine labeling mechanism. An arginine guanidinium can be acylated (e.g., labeled with an NHS ester with the aid of Barton's base). This example reaction may show cross-reactivity or interference by primary amines (e.g., N-terminus, lysine) or thiols (e.g., cysteine), and thus may be performed after N-terminal support immobilization and cysteine and lysine labeling in order to prevent or diminish cross-reactivity.

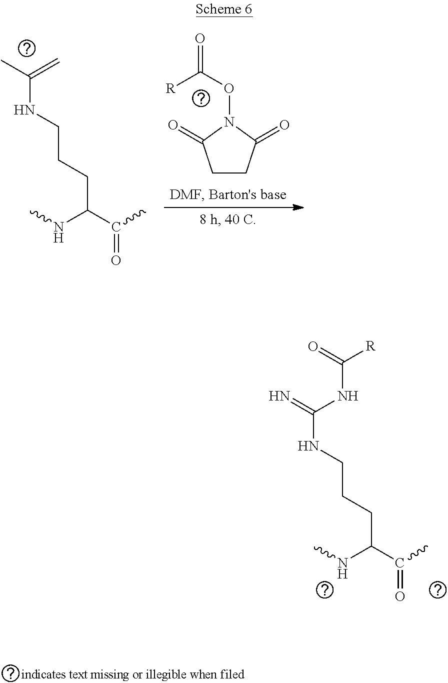

Methionine comprises a relatively low nucleophilicity and can often be selectively labeled by a redox based scheme where an oxaziridine group reacts specifically with a methionine thioether without cross-reacting with cysteine (Scheme 7). The bond formed is stable to reducing agents such as TCEP.

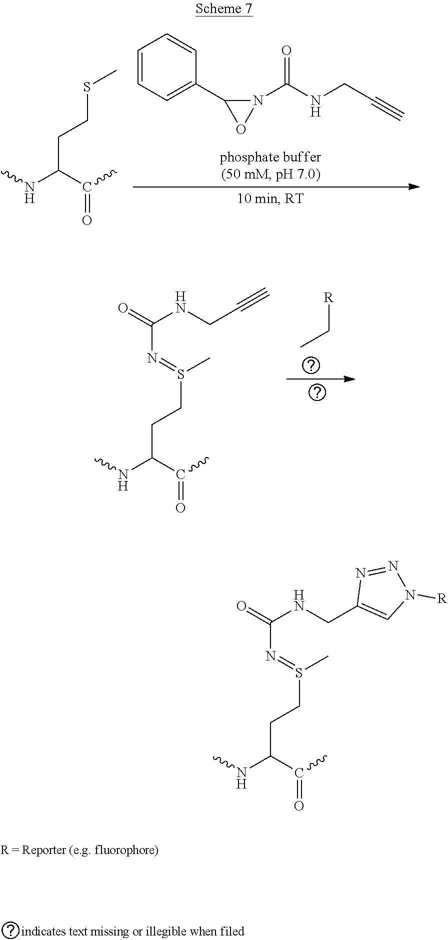

Scheme 8 provides an example of a tryptophan labeling scheme. A tryptophan indole may couple to a diazopropanoate ester, yielding a tertiary amine derivatized tryptophan, The coupling may be metal-catalyst mediated, for example by a dirhodamine(II) tetraacetate complex, which may enhance the selectivity for tryptophan over other amino acid types.

### Scheme 8

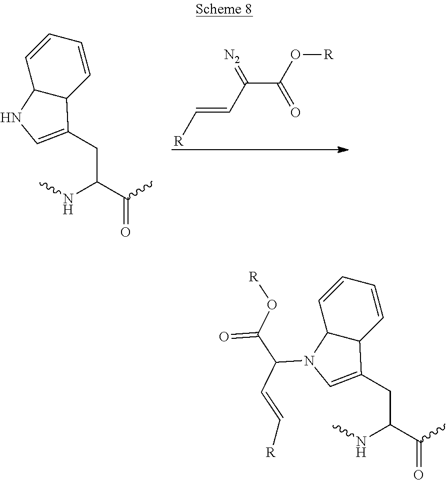

Phosphorylated amino acids such as phosphoserine, phosphotyrosine, or phosphothreonine can be selectively labeled. Such a labeling method may distinguish between types of phosphorylated amino acids. For example, Scheme 9 below provides a phosphoryl beta-elimination followed by a label conjugate addition (e.g., a Michael acceptor reaction) step for selectively labeling of phosphoserine (pSer) and phosphothreonine (pThr) over other phosphorylated amino acids such as phosphotyrosine (pTyr). A subsequent pan-phospho labeling method can be implemented to label pTyr.

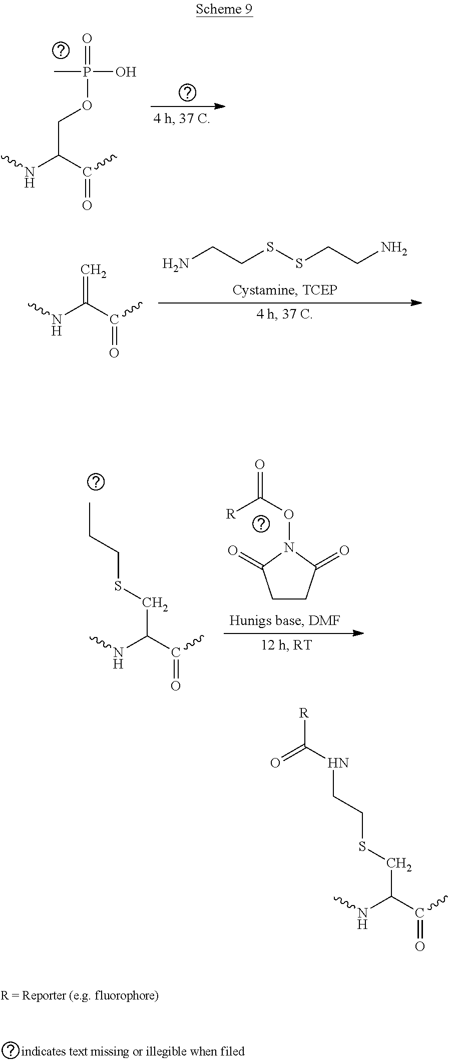

### Peptide Degradation

The present disclosure provides a range of chemical and enzymatic techniques for mild and sequential protein degradation. Degradation can be utilized in a range of peptide sequencing and analysis methods, for example to determine the order or identity of particular amino acids in a fluorosequencing assay. A peptide or protein may be iteratively subjected to cleavage conditions to determine the sequence of at least a portion of its sequence. The entire sequence of a peptide may be determined using the methods and compositions described herein. Controlled amino acid removal (e.g., N- or C-terminal amino acid removal) may be carried out through a variety of techniques including, for example, degradation, organophosphate degradation, or proteolytic cleavage. In some instances, the N-terminal amino acid residue is selectively removed from a peptide. In some instances, the C-terminal amino acid residue is selectively removed from a peptide. A chemical or enzymatic technique for removing a terminal amino acid may remove a defined number of (e.g., exactly one, exactly two, at most two) amino acids. Accordingly, a method for analyzing a peptide may comprise successive degradation and analysis steps, such that the removal of a defined number of amino acids from an N-terminus or C-terminus per step provides position and sequence specific amino acid identifications during analysis. A chemical or enzymatic technique for removing a terminal amino acid may cleave a peptide at a defined location (e.g., only in between two alanine residues, or only at the peptide bond connecting an N-terminal amino acid to the remainder of a peptide).

A terminal amino acid removal method may comprise chemically functionalizing a peptide N-terminus or C-terminus (e.g., with a reagent comprising a structure of Formula (I) or (II)), and then contacting the functionalized terminal amino acid with a reagent (e.g., a hydrazine), a condition (e.g., a high or low pH or temperature), or an enzyme (e.g., an enzyme with specificity for the functionalized terminal amino acid), or with light to remove the functionalized terminal amino acid.

A cleavage method (e.g., a cleavage method implemented within a sequencing method) may comprise enzymatic cleavage. For example, prior to fluorosequencing, a peptide may be subjected to enzymatic cleavage to generate a plurality of shorter peptide fragments. The cleavage method may comprise the use of a single protease, a series of proteases (e.g., provided in a specific order), or a combination of proteases. Exemplary proteases and their associated cleavage sites are provided in TABLE 1.

TABLE 1
Exemplary Proteases

| Protease | Cleavage Site |
|---|---|
| Carboxypeptidase A | C-terminal exopeptidase |
| Carboxypeptidase B | C-terminal exopeptidase; specific for lysine or arginine |
| Carboxypeptidase P | C-terminal exopeptidase |
| Carboxypeptidase Y | C-terminal exopeptidase |
| Cathepsin C | N-terminal exopeptidase; removes N-terminal dipeptide (except |
| | when N-terminal amino acid is lysine or arginine, or when 2^{nd} or |
| | 3^{rd} amino acid from N-terminal is proline) |
| Chymotrypsin | C-terminal exopeptidase; specific for phenylalanine, |
| | tryptophan, and tyrosine |
| Clostripain | arginine |
| Elastase | Alanine, Valine, Serine, Glycine, Leucine, or Isoleucine |
| Endoproteinase Arg-C | Arginine |
| Endoproteinase Glu-C | Glutamic Acid |
| Endoproteinase Lys-C | Lysine |
| Glutamyl endopeptidase | Glutamic acid |
| Kallikrein (Plasma) | Lysine or Arginine |
| Papain | Lysine or Arginine followed by a hydrophobic residue |
| Pepsin | Leucine, Phenylalanine, Tryptophan or Tyrosine |
| Proteinase K | Aliphatic and aromatic amino acids |
| Subtilisin | Hydrophobic amino acids |
| TEV Protease | Specific for the sequences Glutamic acid-Asparagine-Leucine- |
| | Tyrosine-Phenylalanine-Glutamine-Glycine and Glutamic acid- |
| | Asparagine-Leucine-Tyrosine-Phenylalanine-Glutamine-Serine; |
| | cleaves between Glutamine-Glycine or Glutamine-Serine |
| Thermolysin | Isoleucine, Methionine, Phenylalanine, Tryptophan, Tyrosine, |
| | or Valine |
| Trypsin | Lysine or Arginine |

Peptide cleavage may comprise chemical cleavage. Examples of chemical cleavage reagents consistent with the present disclosure include cyanogen bromide, BNPS-skatole, formic acid, hydroxylamine, and 2-nitro-5-thiocyanobenzoic acid. A peptide barcode may comprise a chemically cleavable moiety, such as a disulfide. A peptide barcode may be coupled to a molecule by a linker which comprises a chemically cleavable moiety. A peptide barcode may be coupled to a molecule by a chemically cleavable bond. A cleavage method may comprise a combination (e.g., parallel or sequential use) of chemical and enzymatic cleavage reagents. A cleavage method may comprise activating (e.g., functionalizing) an amino acid for chemical or enzymatic cleavage. For example, a method may comprise derivatizing an N-terminal amino acid residue of a peptide, and then contacting the peptide with an enzyme configured to remove the derivatized N-terminal amino acid residue.

Peptide cleavage conditions may be achieved with a solvent. The solvent may be an aqueous solvent, an organic solvent, or a combination or mixture thereof. The solvent may be an organic solvent. The solvent may comprise dimethylsulfoxide (DMSO). The organic solvent may comprise a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethylsulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and a C₁-C₁₂ haloalkane. The mixture of two or more solvents may be a mixture of DMF and DCM. The mixture of solvents may be any combination thereof.

A degradation process may comprise a plurality of steps. For example, a method may comprise an initial step for derivatizing a terminal amino acid of a peptide, and a subsequent step for cleaving the derivatized terminal amino acid from the peptide. One such method comprises organophosphorus compound-mediated N-terminal functionalization and removal, and thus provides an alternative to the isothiocyanate (e.g., phenyl isothiocyanate) based processes of some degradation schemes.

A cleavage method may comprise digesting a peptide to generate fragments of a desired average length. The cleavage method may generate peptides (e.g., by acting upon a complex mixture of peptides, such as cell lysate) with an average length of at least about 5 amino acids, at least about 8 amino acids, at least about 10 amino acids, at least about 12 amino acids, at least about 15 amino acids, at least about 20 amino acids, at least about 25 amino acids, at least about 30 amino acids, at least about 40 amino acids, or at least about 50 amino acids. The cleavage method may generate peptides with an average length of at most about 50 amino acids, at most about 40 amino acids, at most about 30 amino acids, at most about 25 amino acids, at most about 20 amino acids, at most about 15 amino acids, at most about 12 amino acids, at most about 10 amino acids, at most about 8 amino acids, or at most about 5 amino acids. The cleavage method may generate peptide fragments with an average length of between about 5 and about 20 amino acids, between about 5 and about 30 amino acids, between about 10 and about 20 amino acids, between about 10 and about 30 amino acids, between about 12 and about 18 amino acids, between about 15 and about 30 amino acids, between about 20 and about 40 amino acids, or between about 30 and about 50 amino acids.

A reaction mixture may comprise a stoichiometric or an excess concentration of a cleavage compound (e.g., relative to the concentration of peptides to be cleaved). The reaction mixture may comprise at least about 0.001% v/v, at least about 0.01% v/v, at least about 0.1% v/v, at least about 1% v/v, at least about 5% v/v, at least about 10% v/v, at least about 15% v/v, at least about 20% v/v, at least about 30% v/v, at least about 40% v/v, at least about 50% v/v, or more of the cleavage compound. The reaction mixture may comprise at most about 50% v/v, at most about 40% v/v, at most about 30% v/v, at most about 20% v/v, at most about 15% v/v, at most about 10% v/v, at most about 5% v/v, at most about 1% v/v, at most about 0.1% v/v, at most about 0.01% v/v, at most about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound. The reaction mixture may comprise about 5% v/v of the cleavage compound.

The reaction may be performed at a temperature of at least about 0° C., at least about 5° C., at least about 10° C., at least about 15° C., at least about 20° C., at least about 25° C., at least about 30° C., at least about 40° C., at least about 50° C., at least about 60° C., at least about 70° C., at least about 80° C., or at least about 90° C. The reaction may be performed at a temperature of at most about 90° C., at most about 80° C., at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature. The reaction may be performed at close to 0° C. or below 0° C. (e.g., in the presence of an antifreeze).

The peptide and the cleavage compound may be mixed or incubated for at least about 1 minute, at least about 5 minutes, at least about 10 minutes, at least about 20 minutes, at least about 30 minutes, at least about 40 minutes, at least about 50 minutes, at least about 60 minutes, at least about 2 hours, at least about 3 hours, at least about 4 hours, at least about 6 hours, at least about 8 hours, at least about 10 hours, at least about 12 hours, at least about 16 hours, at least about 20 hours, at least about 24 hours, or more. The peptide and the cleavage compound may be mixed or incubated for at most about 24 hours, at most about 20 hours, at most about 16 hours, at most about 12 hours, at most about 10 hours, at most about 8 hours, at most about 6 hours, at most about 4 hours, at most about 3 hours, at most about 2 hours, at most about 1 hour, at most about 50 minutes, at most about 40 minutes, at most about 30 minutes, at most about 20 minutes, at most about 10 minutes, at most about 5 minutes, at most about 1 minute, or less. The peptide and the cleavage compound may be mixed or incubated from about 1 minute to about 24 hours, 5 minutes to about 6 hours, 5 minutes to about 2 hours, or 5 minutes to about 30 minutes.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (such as peptides, nucleic acids, lipids, or small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffycoat proteins may be partitioned and separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and analyzing the peptides by a fluorosequencing method of the present disclosure. A method may comprise capturing peptides from a sample using a C-terminal capture reagent, and analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and a peptides from a human plasma sample, sequencing the cell free DNA (e.g., to identify a cancer marker), and performing proteomic analysis on the plasma proteins.

### Computer Systems

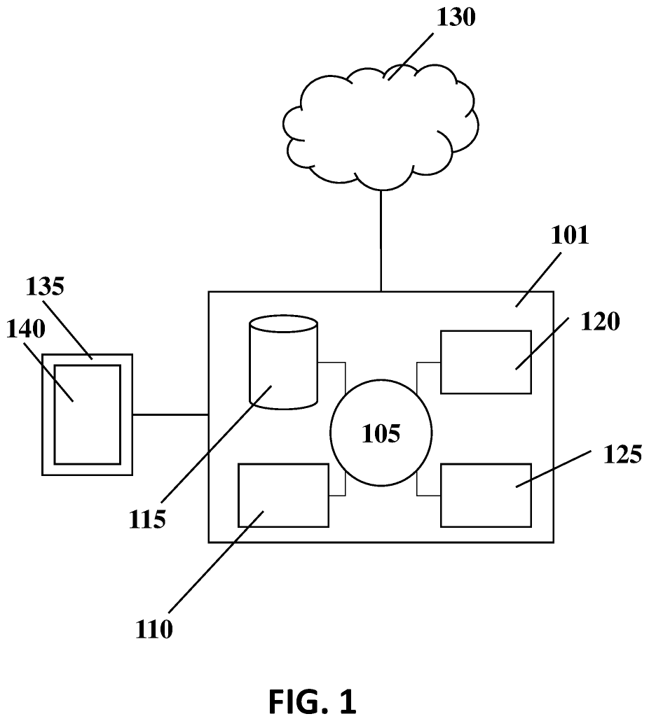

The present disclosure provides computer systems that are programmed to implement methods of the disclosure. FIG. 1 shows a computer system 101 that is programmed or otherwise configured to implement methods or parts of methods disclosed herein, including compiling, analyzing, and displaying data obtained through the present methods. The computer system 101 may regulate various aspects of the present disclosure, such as, for example, controlling cell partitioning and optical imaging devices. The computer system 101 may be an electronic device of a user or a computer system that is remotely located with respect to the electronic device. The electronic device may be a mobile electronic device.

The computer system 101 includes a central processing unit (CPU, also “processor” and “computer processor” herein) 105, which may be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some cases is a telecommunication and/or data network. The network 130 may include one or more computer servers, which may enable distributed computing, such as cloud computing. The network 130, in some cases with the aid of the computer system 101, may implement a peer-to-peer network, which may enable devices coupled to the computer system 101 to behave as a client or a server.

The CPU 105 may execute a sequence of machine-readable instructions, which may be embodied in a program or software. The instructions may be stored in a memory location, such as the memory 110. The instructions may be directed to the CPU 105, which may subsequently program or otherwise configure the CPU 105 to implement methods of the present disclosure. Examples of operations performed by the CPU 105 may include fetch, decode, execute, and writeback.

The CPU 105 may be part of a circuit, such as an integrated circuit. One or more other components of the system 101 may be included in the circuit. In some cases, the circuit is an application specific integrated circuit (ASIC).

The storage unit 115 may store files, such as drivers, libraries and saved programs. The storage unit 115 may store user data, e.g., user preferences and user programs. The computer system 101 in some cases may include one or more additional data storage units that are external to the computer system 101, such as located on a remote server that is in communication with the computer system 101 through an intranet or the Internet.

The computer system 101 may communicate with one or more remote computer systems through the network 130. For instance, the computer system 101 may communicate with a remote computer system of a user (e.g., a fluorimeter or a cell sorting device). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user may access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine executable or machine readable code may be provided in the form of software. During use, the code may be executed by the processor 105. In some cases, the code may be retrieved from the storage unit 115 and stored on the memory 110 for ready access by the processor 105. In some situations, the electronic storage unit 115 may be precluded, and machine-executable instructions are stored on memory 110.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 may include or be in communication with an electronic display 135 that comprises a user interface (UI) 140 for providing, for example, orders and options for controlling flow rates in a cell sorting device. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 105. The algorithm may, for example, determine a correlation using linear and quadratic discriminant analysis (LDA and QDA), Support Vector Machine (SVM), linear discriminant analysis (LDA), quadratic discriminant analysis (QDA), Naive Bayes, Random Forest, or any other suitable method.

Compounds disclosed herein may be made by the methods depicted in the reaction schemes shown below. Procedures are provided herein that, in combination with the knowledge of the synthetic organic chemist of ordinary skill in the art, are in some embodiments used to prepare the full range of compounds as disclosed and claimed herein.

### EXAMPLES

### Example 1—Synthesis of a Carbodiimide N-Terminal Coupling Reagent

### Scheme 10—Preparation of a Carbodiimide Reagent

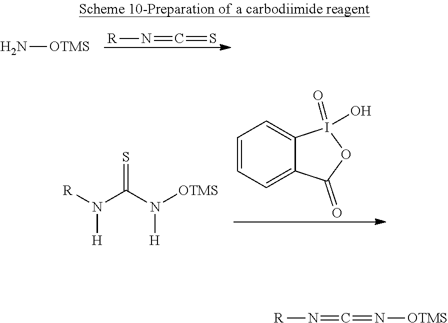

An example of a synthetic route for a carbodiimide degradation reagent is provided in Scheme 10. Briefly, 1 mmol O-(trimethylsilyl) hydroxylamine is mixed with 1 mmol isothiocyanate in acetonitrile at 70° C. for 6 hours, yielding a thiourea product. The mixture is cooled, and the solid is filtered with cold ether washes before recrystallization in dimethylformamide. The powder is redissolved in DMSO with 3 mmol 2-iodoxybenzoic, and stirred overnight at room temperature. The reaction is quenched by addition of propanol, and the resulting white powder is collected by filtration with hexane washes.

### Example 2—Preparation of N-Terminal Coupling Agent

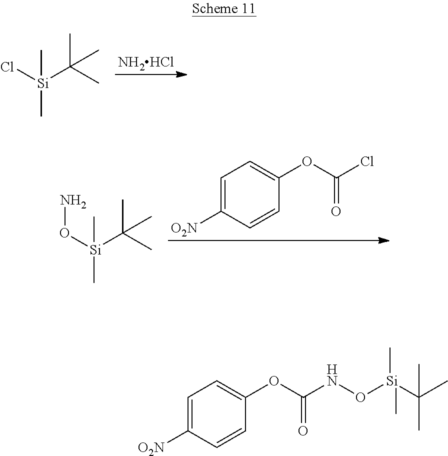

Step 1. Synthesis of O-(tert-butyldimethylsilyl) hydroxylamine: To a solution of hydroxylamine hydrochloride (1.39 g, 20.0 mmol) stirring in dry DCM (15 ml), anhydrous ethylenediamine (1.33 g, 20.0 mmol) was added and the resulting mixture was stirred at room temperature for 24 h. Then a solution of TBDMSCl (3.01 g, 20.0 mmol) in dry DCM (5 mL) was added via cannula. The reaction mixture was stirred for 48 h and filtered to remove the solids, which were washed with DCM. The solvent was evaporated in vacuo, and upon cooling in an ice bath, a waxy white solid (2.55 g, 87%) was obtained as the desired product.

Step 2. Synthesis of 4-nitrophenyl ((tert-butyldimethylsilyl)oxy) carbamate: (O-(tert-butyldimethylsilyl) hydroxylamine (2.50 g, 17 mmol) was added in a mixture of dry pyridine (1.34 g, 17 mmol) and dry DCM (25 mL) followed by dropwise addition of a solution of 4-nitrophenyl chloroformate (3.42 g, 17 mmol) in dry DCM (5 mL) over 15 min. The resulting mixture was stirred at room temperature for 24 h. Then reaction mixture was diluted with DCM; washed with water, saturated aqueous solution of sodium bicarbonate (2×), and brine; dried over sodium sulfate; and concentrated under reduced pressure. The crude product was purified by chromatography (90 g silica gel, 10% EtOAc/hexanes eluent) to obtain a white solid (3.70 g, 70%) as the desired product.

### Example 3—Base-Induced N-Terminal Degradation

N,N′-bis (tert-butoxycarbonyl)-1H-pyrazole-1-carboxamidine was used to functionalize peptides, and 2% NaOH was used to cleave the terminal amino acid of the resulting functionalized peptide. A base-induced cleavage method was used to afford a functionalized peptide, as shown in Scheme 12.

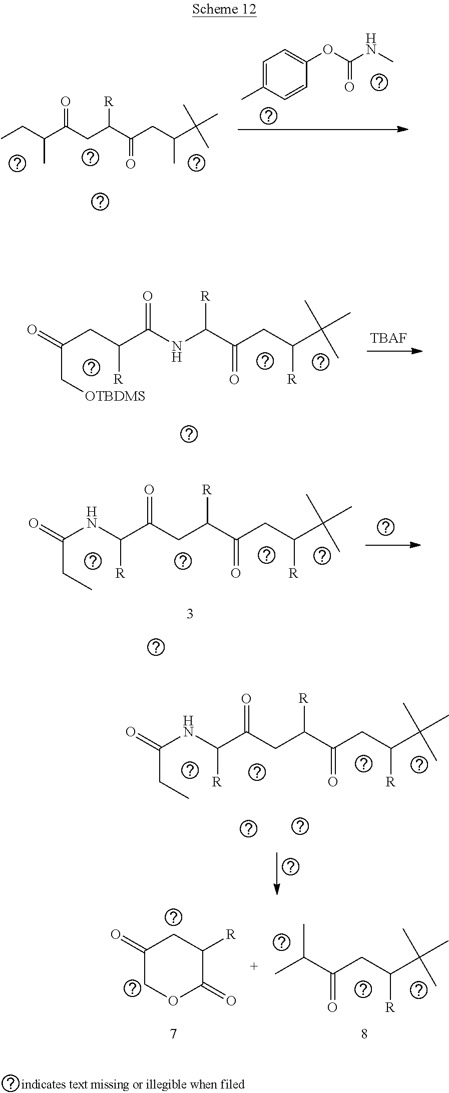

Reagent 2, 4-nitrophenyl ((tert-butyldimethylsilyl)oxy) carbamate, comprises a p-nitrophenyl leaving group that reacts with the N-terminal of peptides in nearly quantitative yield. Reagent 2 also comprises a TBDMS-protected oximate, which upon mild deprotection with tetrabutyl ammonium fluoride (TBAF) generates a hydroxamic urea, which is a super nucleophile due to the a-effect. In the last step, the functionalized peptide is treated with a base to result in cyclized product 7 and leaving a new N-terminus on a truncated peptide for another cycle of sequencing.

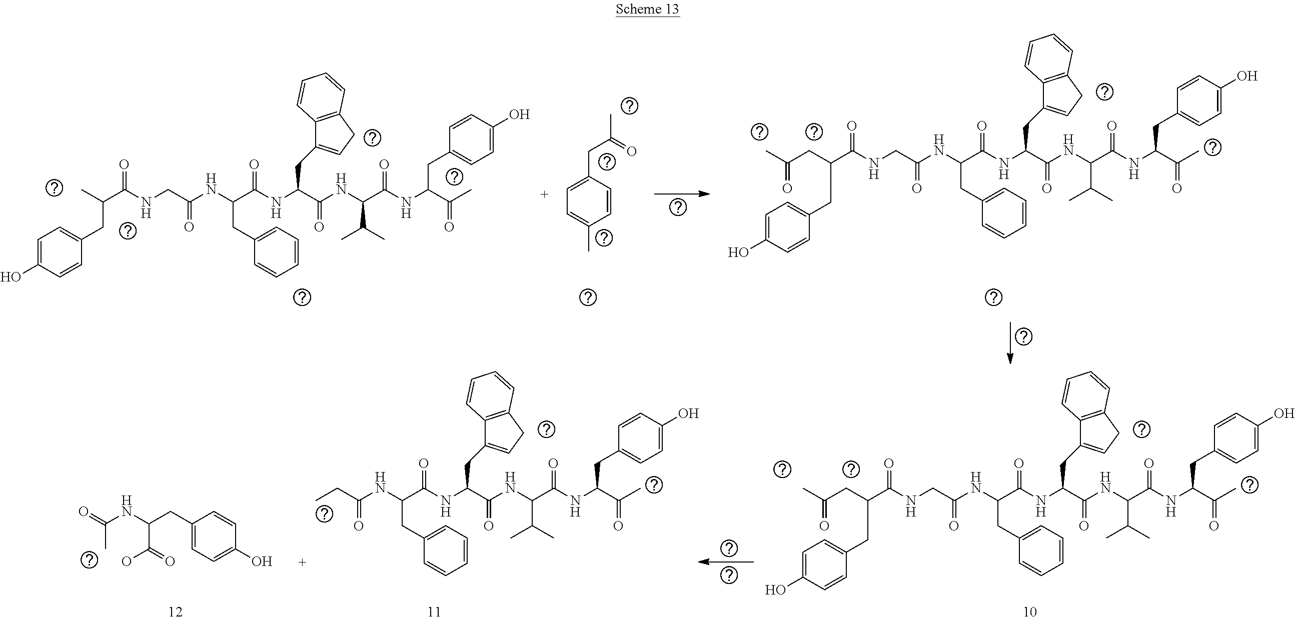

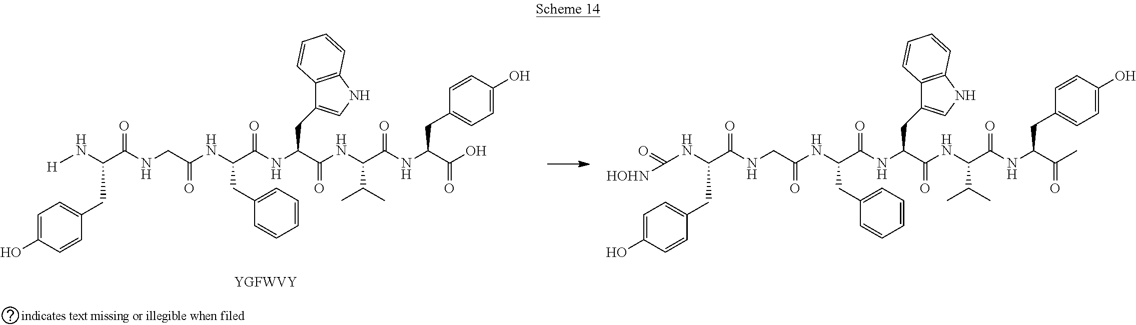

To evaluate the effectiveness of the base-induced N-terminal degradation reaction, several peptides with prepared with varying N-terminal and N-terminal adjacent amino acid residues using the 20 canonical amino acids (Scheme 13 and 14).

Functionalization of peptide: To the solution of peptide (1.0 equiv.) in dimethylformamide (DMF) was added 4-nitrophenyl ((tert-butyldimethylsilyl)oxy) carbamate (1.0 equiv.). The reaction was stirred at room temperature for 2-5 hours. The progress of the reaction was monitored by LCMS. Upon completion of the reaction, tetrabutylammonium fluoride (1.0 equiv.) was added, and the resulting mixture was stirred further for 2 hours and monitored by LCMS. The resulting mixture was purified by preparative reverse phase high-performance liquid chromatography (RP-HPLC) to obtain the desired functionalized peptide.

Sequencing: To a 20 μL aliquot of a purified functionalized peptide solution (2 mM) dissolved in a acetonitrile water mixture (1:1) was added in 100 μL 1% Ba(OH)₂ solution. The mixture was stirred at 37° C. for 5 hours. Upon completion of the reaction, the reaction was quenched using aqueous acetic acid, and the mixture was analyzed by LCMS.

To increase the yield of truncated peptides, reaction times, temperature, solvents, base concentrations were modified to vary the a-effect nucleophile to be a hydrazines or aminoethers. The truncated peptide was obtained in different yields depending upon the N-terminal amino acid.

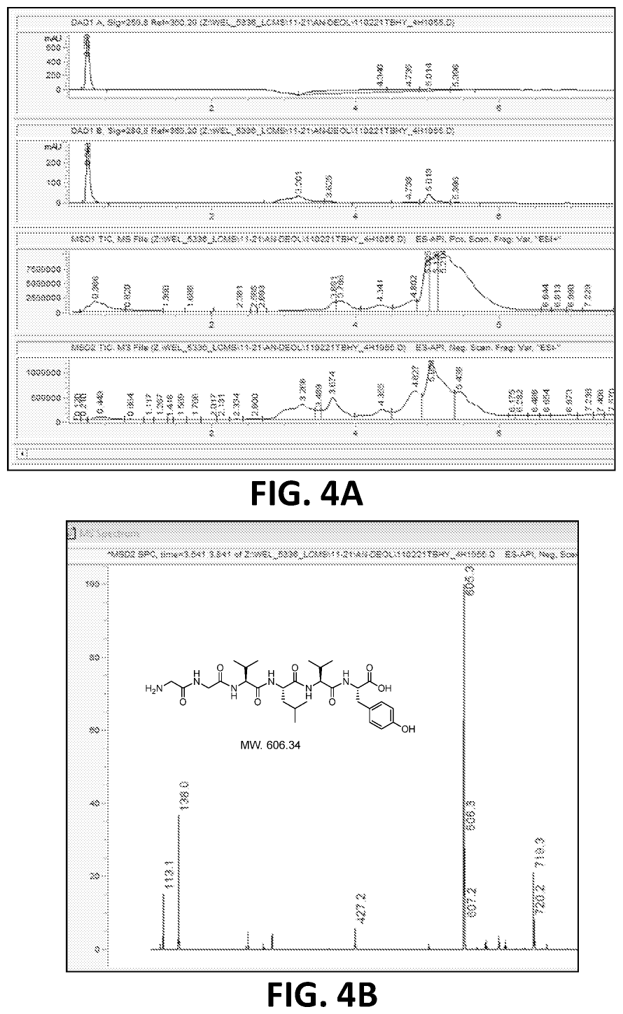

The functionalized peptides were subjected to different bases (Table 2). The highest reaction yields (50-70%) of truncated peptides were observed with the use of 1% Ba(OH)₂. Importantly, the heterocyclic side product (Product 7, above) was identified. Cs₂CO₃, NMP, DBU, and NaOH did not yield a desirable amount of the degraded peptide. Scheme 15 shows peptide degradation using 4-nitrophenyl ((tert-butyldimethylsilyl)oxy) carbamate as a labeling agent and hydrazine hydride as a base. FIG. 4A-4B show that sequencing of a functionalized peptide with a phenylalanine at the N-terminus produced the desired degraded peptide product after treatment with Tris buffer (pH 9.5) at 50° C. for 2 h.

TABLE 2

| Base | Time | % Yield of Truncated peptide (approx.) |
|---|---|---|
| Cs₂CO₃ | 2 h | <10% |
| K₂CO₃ | 2 h | <10% |
| NMP | 2 h | 0 |
| DBU | 2 h | 0 |
| 0.7M Tris buffer (pH 9.5) | 2 h | <10% |
| Buffer (pH 13) | 2 h | <20% |
| 2% NaOH | 2 h | <20% |
| 1% NaOH | 2 h | <20% |
| 1% Ba(OH)₂ | 2 h | <40% |
| 1% Ba(OH)₂ | 2 h | 70% |

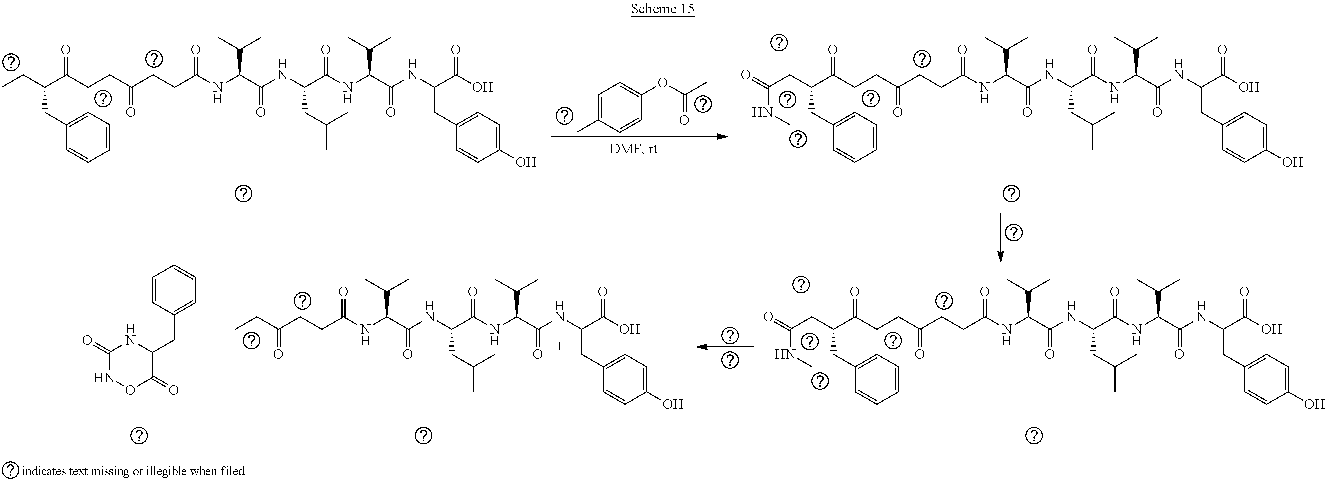

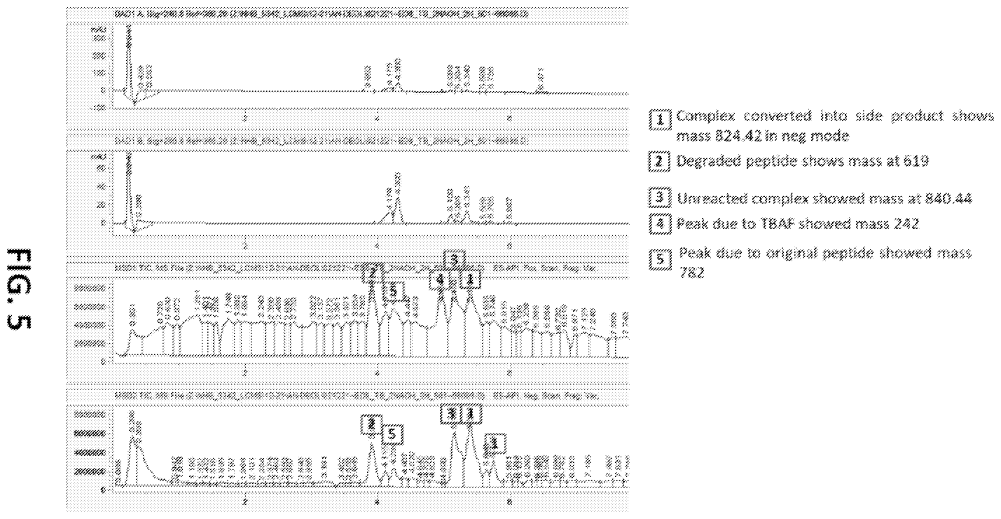

NaOH-mediated degradation Treatment of the functionalized peptide with 2% NaOH at 50° C. for 5 h was tested to determine the efficacy of peptide cleavage. A peptide comprising tyrosine at the N-terminus (YFAVALV; SEQ ID NO: 12) was treated with 2% NaOH at 50° C. for 5 h. Mass spectrometry analysis demonstrated that the yield of the cleaved peptide was 23%. The yield of reaction remained the same for the peptide when treated with 1% NaOH at 50° C. FIG. 5 shows LC-MS analysis of a peptide comprising an N-terminal tyrosine upon treatment with 2% NaOH at 50° C. for 5 h. Detected compounds included: 1) complex converted into side product; 2) degraded peptide; 3) unreacted complex; 4) TBAF; 5) and unreacted peptide.

Ba(OH)₂-mediated degradation: Peptides of Table 3 were treated with 4-nitrophenyl ((tert-butyldimethylsilyl)oxy) carbamate by dissolving the compounds in DMF and mixing the resulting solution at room temperature for 2 h. The functionalized peptides were cleaved by treating the functionalized peptides with 1% Ba(OH)₂ at 37° C. for 5 h.

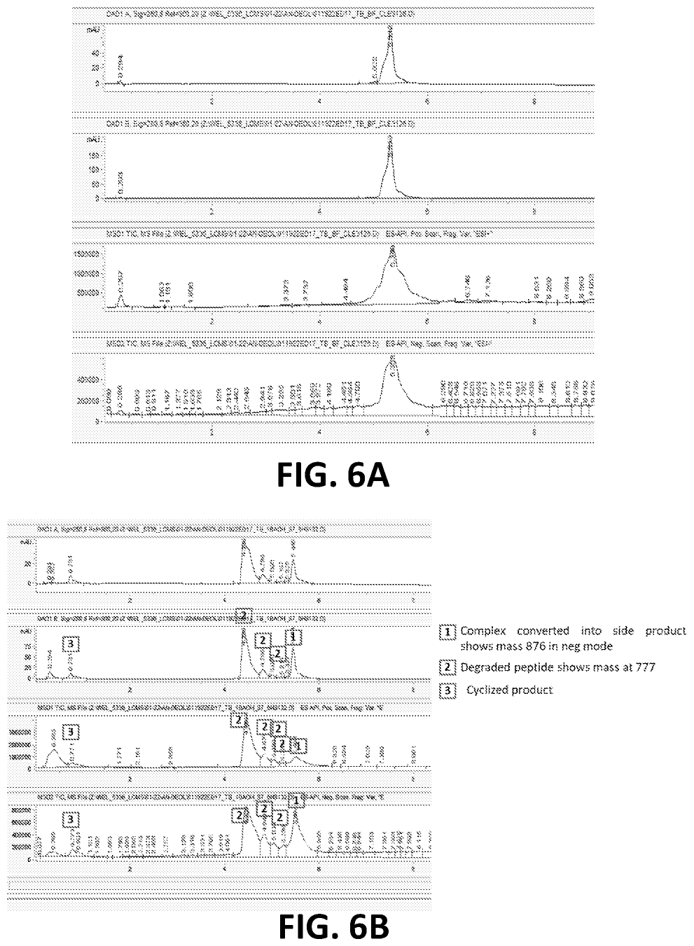

Table 3 shows the yields of truncated peptides obtained using various different peptide sequences. The data showed that N-terminal cleavage of functionalized peptides produced desired products in 40-84% yield. FIG. 6A shows an LC-MS trace of a functionalized peptide (YGFWVY; SEQ ID NO: 1) with a tyrosine residue at the N-terminal before cleavage with Ba(OH)₂. FIG. 6B shows LC-MS data of the degraded peptide after treatment with 1% Ba(OH)₂ at 37° C. for 5 hr, including: 1) complex converted into side product; 2) degraded peptide; and 3) cyclized product.

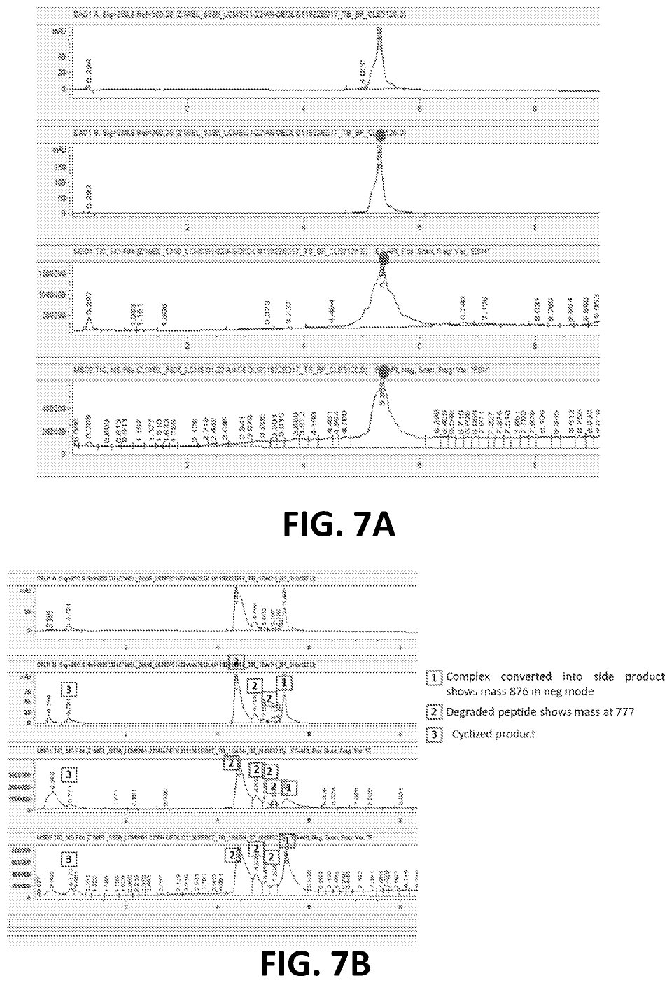

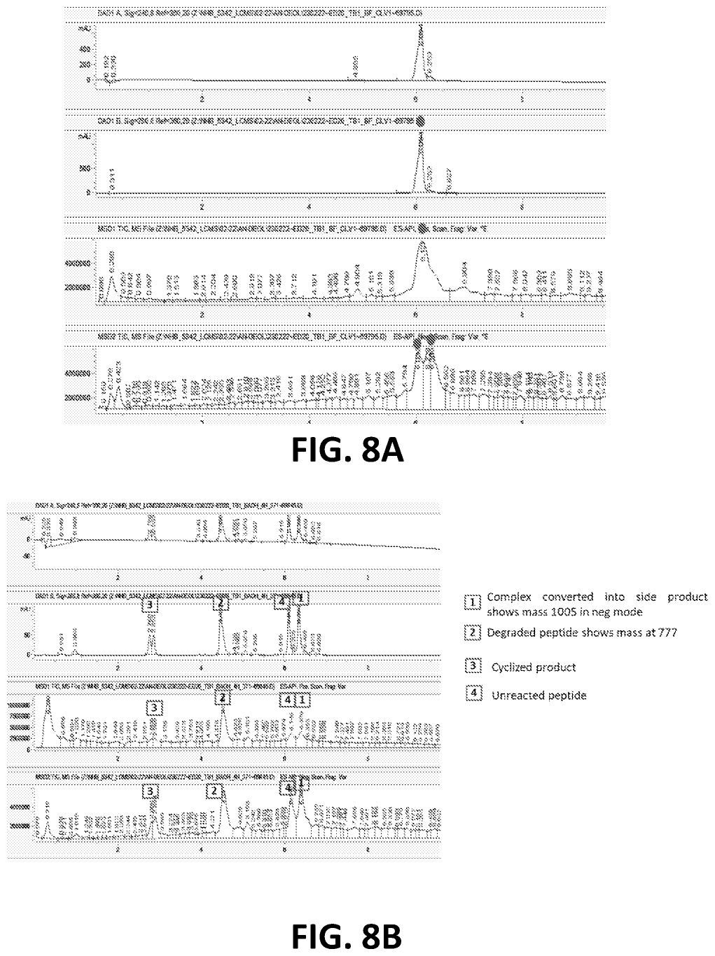

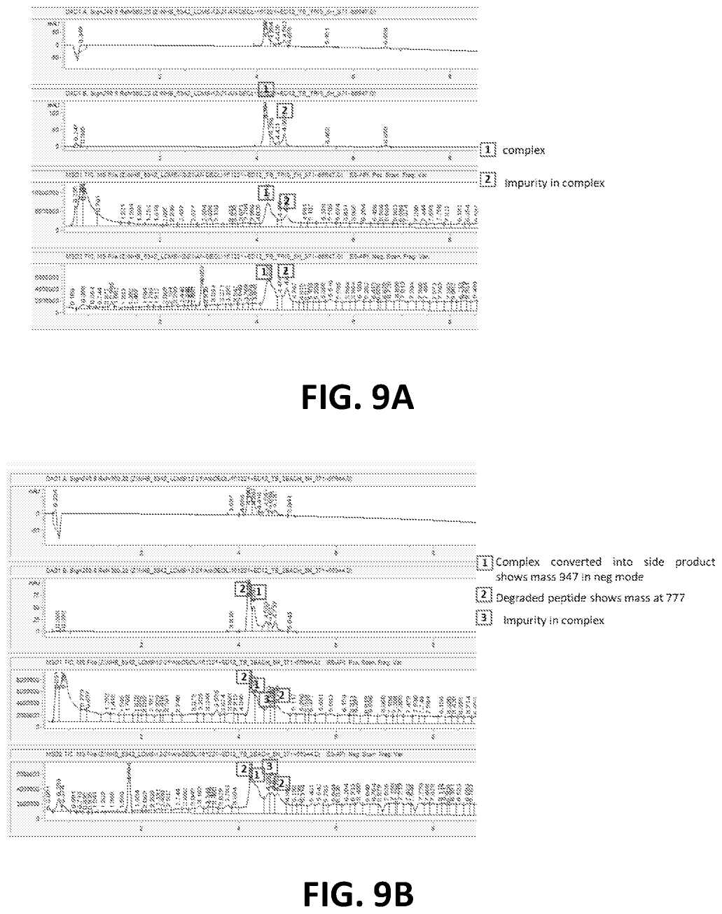

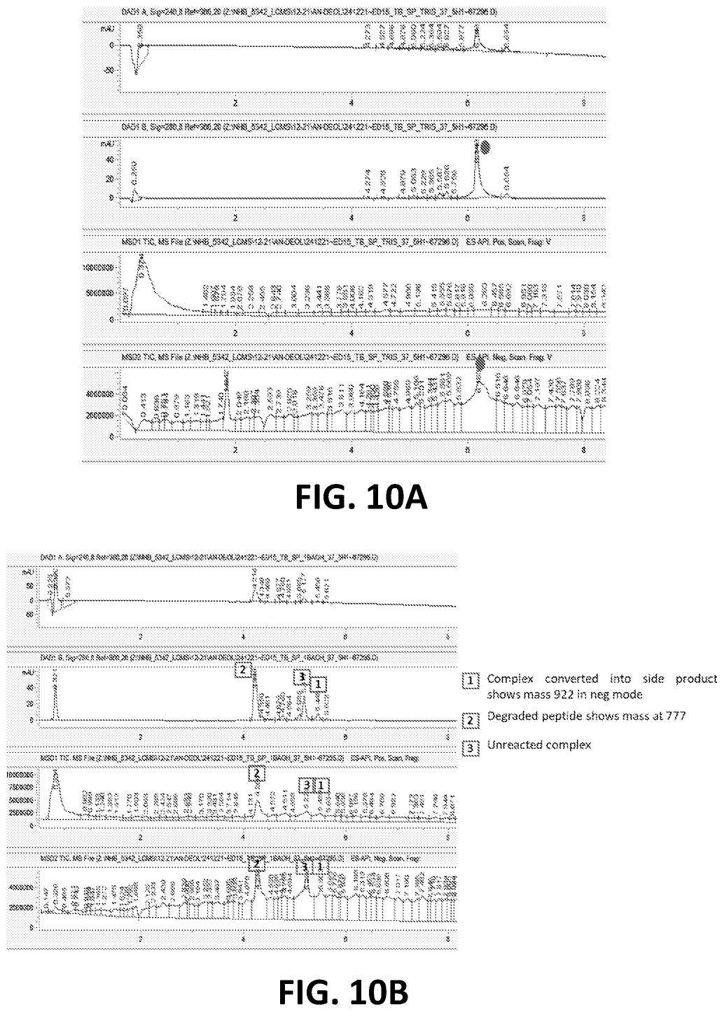

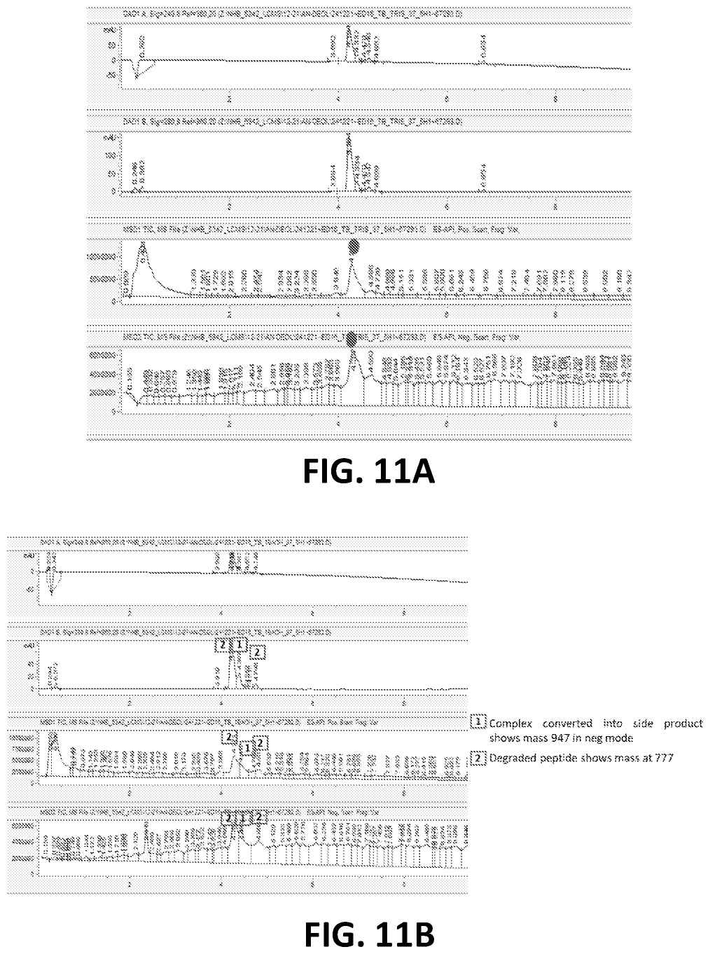

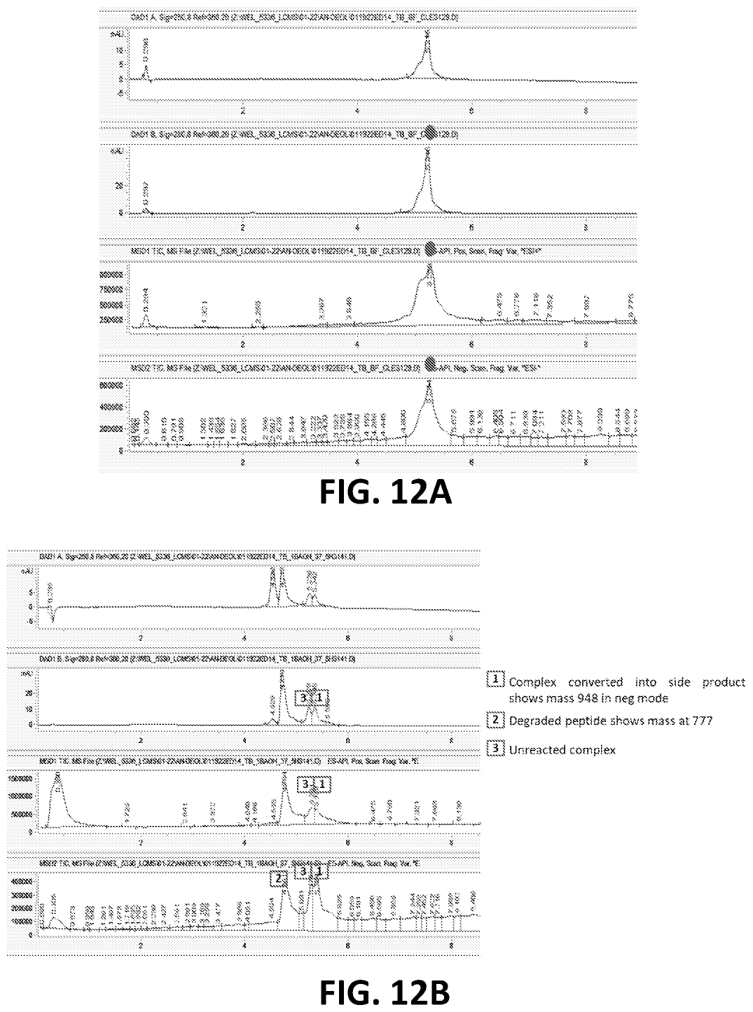

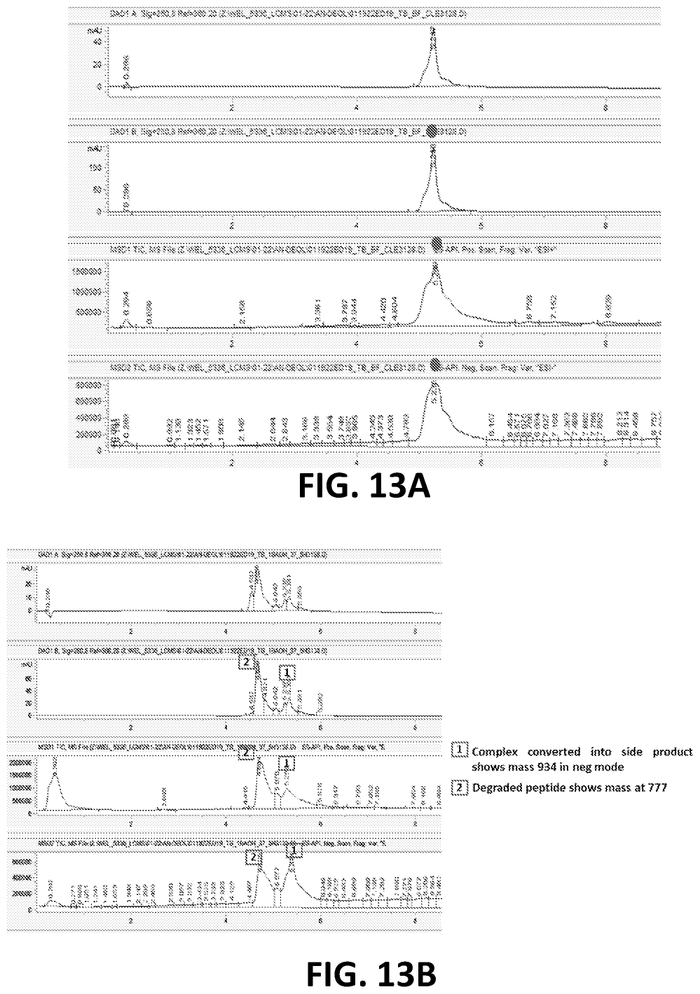

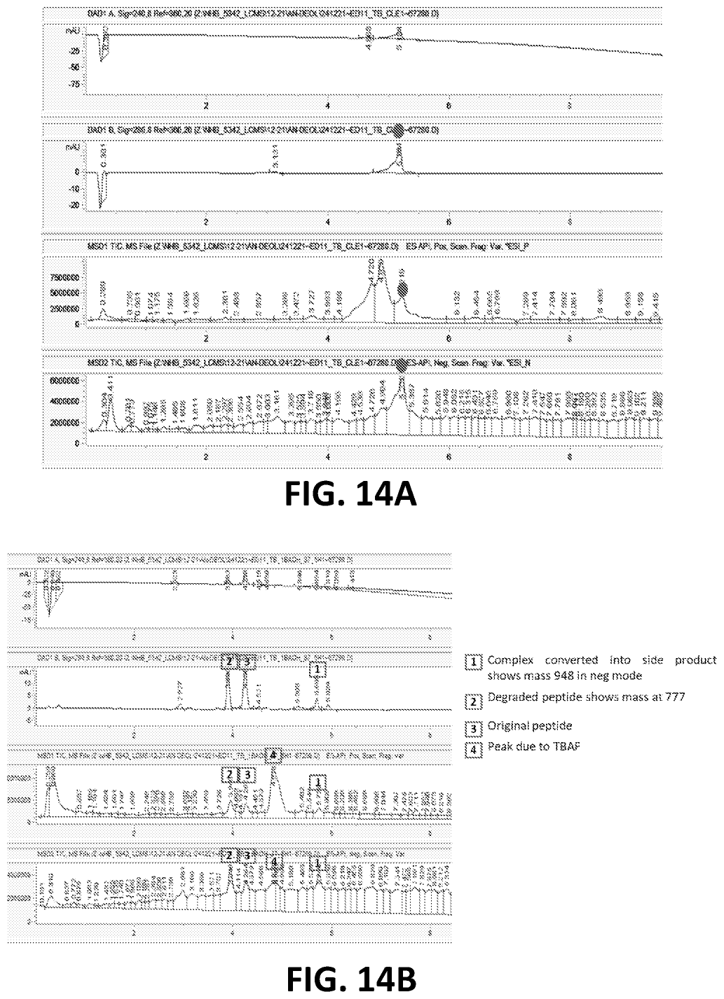

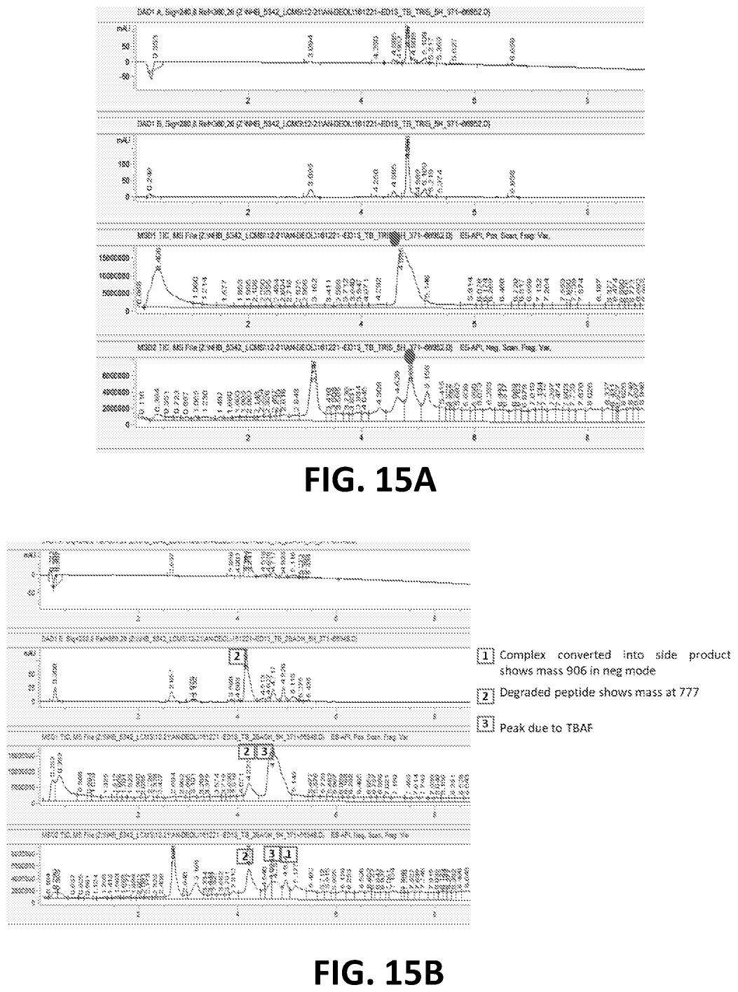

FIG. 7A shows an LC-MS trace of a functionalized peptide (YGFWVY; SEQ ID NO: 1) before cleavage with Ba(OH)₂. FIG. 7B shows LC-MS data of the degraded peptide after treatment with 1% Ba(OH)₂ at 37° C. for 5 hr, including: 1) complex converted into side product; 2) degraded peptide; and 3) cyclized product. The cleaved peptide yield based on DAD1 B spectra was 70%. FIG. 8A shows an LC-MS trace of a functionalized peptide (WYFWYV; SEQ ID NO: 2) with a tryptophan residue at the N-terminal before cleavage with Ba(OH)₂. FIG. 8B shows LC-MS data of the degraded peptide after treatment with 1% Ba(OH)₂ at 37° C. for 5 hr, including: 1) complex converted into side product; 2) degraded peptide; 3) cyclized product; and 4) unreacted product. The cleaved peptide yield based on MSD1 spectra data was 54%. The yield could be increased with an increased reaction time. FIG. 9A shows an LC-MS trace of a functionalized peptide (KYFW; SEQ ID NO: 9) with a lysine residue at the N-terminal before cleavage with Ba(OH)₂. FIG. 9B shows LC-MS data of the degraded peptide after treatment with 1% Ba(OH)₂ at 37° C. for 5 hr, including: 1) complex converted into side product; 2) degraded peptide; and 3) an impurity. The cleaved peptide yield based on DAD1 B spectra was 62%. FIG. 10A shows an LC-MS trace of a functionalized peptide (CYFWVY; SEQ ID NO: 7) with a cysteine residue at the N-terminal before cleavage with Ba(OH)₂. FIG. 10B shows LC-MS data of the degraded peptide after treatment with 1% Ba(OH)₂ at 37° C. for 5 hr, including: 1) complex converted into side product; 2) degraded peptide; and 3) unreacted product. The cleaved peptide yield based on DAD1 B spectra was 65%, which increased with increased reaction times. FIG. 11A shows an LC-MS trace of a functionalized peptide (HYFWVY; SEQ ID NO: 6) with a histidine residue at the N-terminal before cleavage with Ba(OH)₂. FIG. 11B shows LC-MS data of the degraded peptide after treatment with 1% Ba(OH)₂ at 37° C. for 5 hr, including: 1) complex converted into side product; and 2) degraded peptide. The cleaved peptide yield based on DAD1 B spectra was 77%. FIG. 12A shows an LC-MS trace of a functionalized peptide (EYFWVY; SEQ ID NO: 4) with a glutamic acid residue at the N-terminal before cleavage with Ba(OH)₂. FIG. 12B shows LC-MS data of the degraded peptide after treatment with 1% Ba(OH)₂ at 37° C. for 5 hr, including: 1) complex converted into side product; 2) degraded peptide; and 3) unreacted peptide. The cleaved peptide yield based on DAD1 B spectra was 63%, which could be increased with increased reaction times. FIG. 13A shows an LC-MS trace of a functionalized peptide (DYFWVY; SEQ ID NO: 5) with a aspartic acid residue at the N-terminal before cleavage with Ba(OH)₂. FIG. 13B shows LC-MS data of the degraded peptide after treatment with 1% Ba(OH)₂ at 37° C. for 5 hr, including: 1) complex converted into side product; and 2) degraded peptide. The cleaved peptide yield based on DAD1 B spectra was 67%. FIG. 14A shows an LC-MS trace of a functionalized peptide (PFFAYV; SEQ ID NO: 10) with a proline residue at the N-terminal before cleavage with Ba(OH)₂. FIG. 14B shows LC-MS data of the degraded peptide after treatment with 1% Ba(OH)₂ at 37° C. for 5 hr, including: 1) complex converted into side product; 2) degraded peptide; 3) original peptide; and 4) TBAF. The cleaved peptide yield based on DAD1 B spectra was 40%. FIG. 15A shows an LC-MS trace of a functionalized peptide (SYFWVY; SEQ ID NO: 8) with a serine residue at the N-terminal before cleavage with Ba(OH)₂. FIG. 15B shows LC-MS data of the degraded peptide after treatment with 1% Ba(OH)₂ at 37° C. for 5 hr, including: 1) complex converted into side product; 2) degraded peptide; 3) TBAF. The cleaved peptide yield based on DAD1 B spectra was 84%.

TABLE 3

| SEQ | | | |
|---|---|---|---|
| ID | Peptide | | |
| NO. | Sequence | Reaction | Yield |
| 1. | YGFWVY | 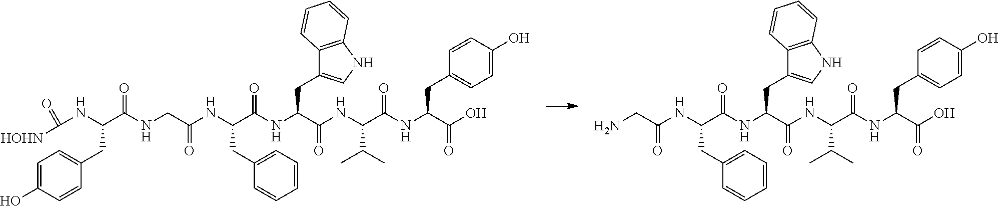 | 70% |
| 2. | WYFWYV | 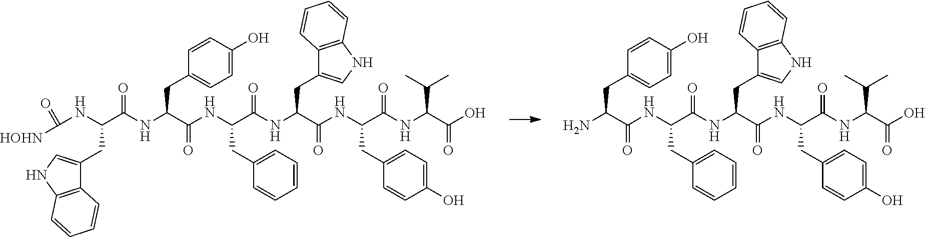 | 54% |
| 3. | QYFWVY | 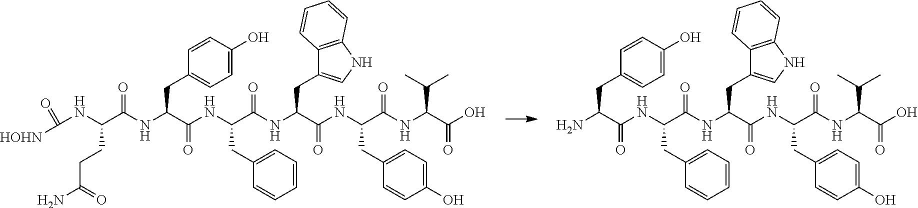 | 52% |
| 4. | EYFWVY | 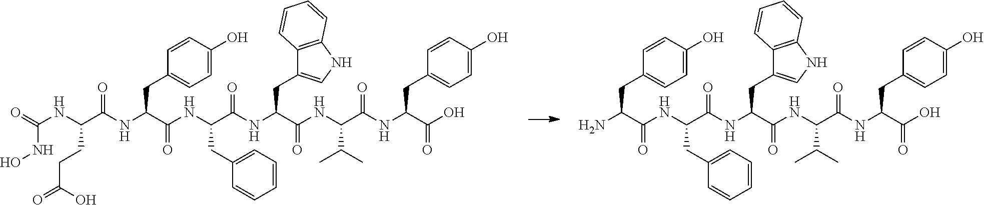 [SMILES: CC(C)[C@H](NC(=O)[C@H](Cc1c[nH]c2ccccc12)NC(=O)[C@H](Cc1ccccc1)NC(=O)[C@H](Cc1ccc(O)cc1)NC(=O)[C@H](CCC(=O)O)NC(=O)NO)C(=O)N[C@@H](Cc1ccc(O)cc1)C(=O)O ; CC(C)[C@H](NC(=O)[C@H](Cc1c[nH]c2ccccc12)NC(=O)[C@H](Cc1ccccc1)NC(=O)[C@@H](N)Cc1ccc(O)cc1)C(=O)N[C@@H](Cc1ccc(O)cc1)C(=O)O] | 63% |
| 5. | DYFWVY | 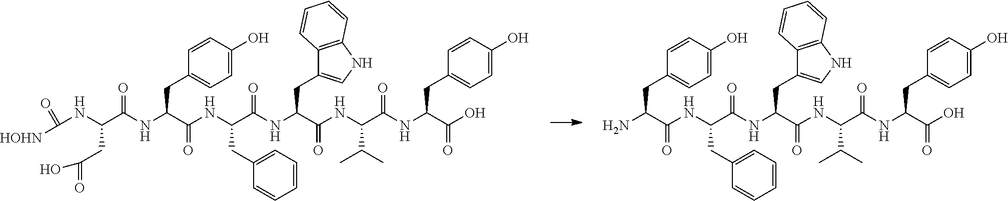 | 67% |
| 6. | HYFWVY | 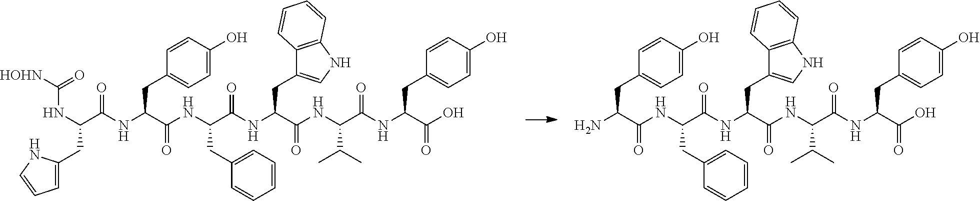 | 77% |
| 7. | CYFWVY | 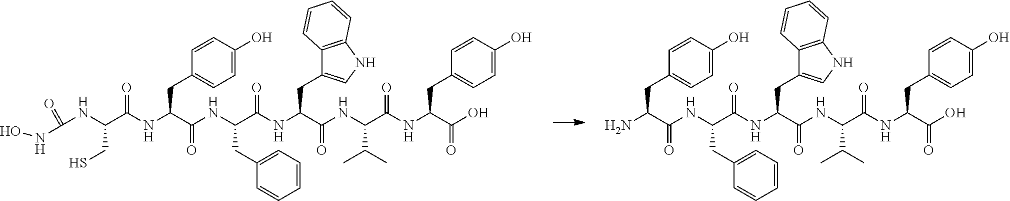 [SMILES: CC(C)[C@H](NC(=O)[C@H](Cc1c[nH]c2ccccc12)NC(=O)[C@H](Cc1ccccc1)NC(=O)[C@H](Cc1ccc(O)cc1)NC(=O)[C@H](CS)NC(=O)NO)C(=O)N[C@@H](Cc1ccc(O)cc1)C(=O)O ; CC(C)[C@H](NC(=O)[C@H](Cc1c[nH]c2ccccc12)NC(=O)[C@H](Cc1ccccc1)NC(=O)[C@@H](N)Cc1ccc(O)cc1)C(=O)N[C@@H](Cc1ccc(O)cc1)C(=O)O] | 65% |
| 8. | SYFWVY | 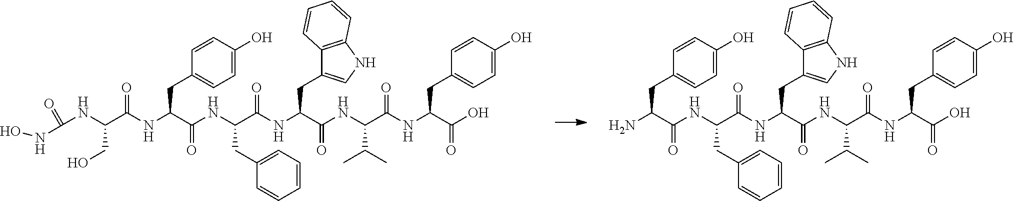 [SMILES: CC(C)[C@H](NC(=O)[C@H](Cc1c[nH]c2ccccc12)NC(=O)[C@H](Cc1ccccc1)NC(=O)[C@H](Cc1ccc(O)cc1)NC(=O)[C@H](CO)NC(=O)NO)C(=O)N[C@@H](Cc1ccc(O)cc1)C(=O)O ; CC(C)[C@H](NC(=O)[C@H](Cc1c[nH]c2ccccc12)NC(=O)[C@H](Cc1ccccc1)NC(=O)[C@@H](N)Cc1ccc(O)cc1)C(=O)N[C@@H](Cc1ccc(O)cc1)C(=O)O] | 84% |
| 9. | KYFW | 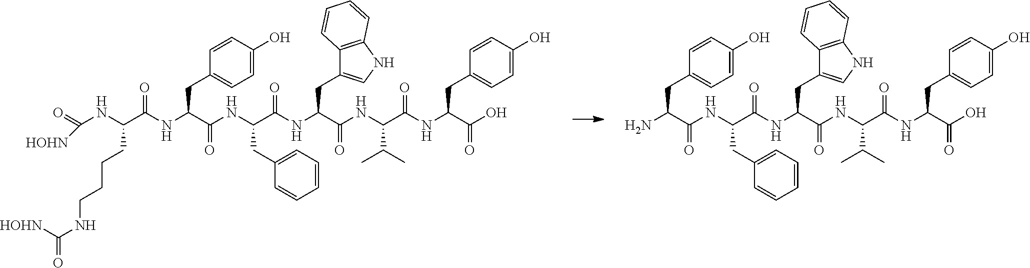 | 62% |
| 10. | PFFAYVY | 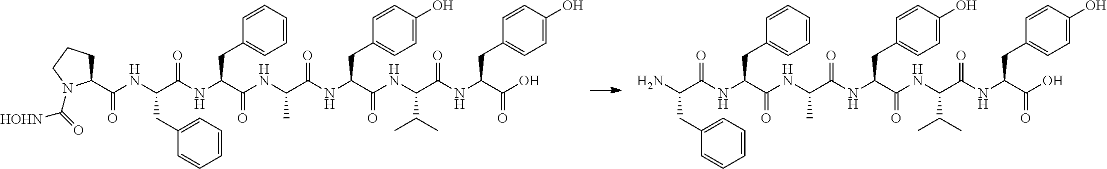 | 40% |

### Example 4-Base-Induced Terminal Degradation Using 1-((9H-fluoren-9-yl)methyl) 2-(4-nitrophenyl) hydrazine-1,2-dicarboxylate

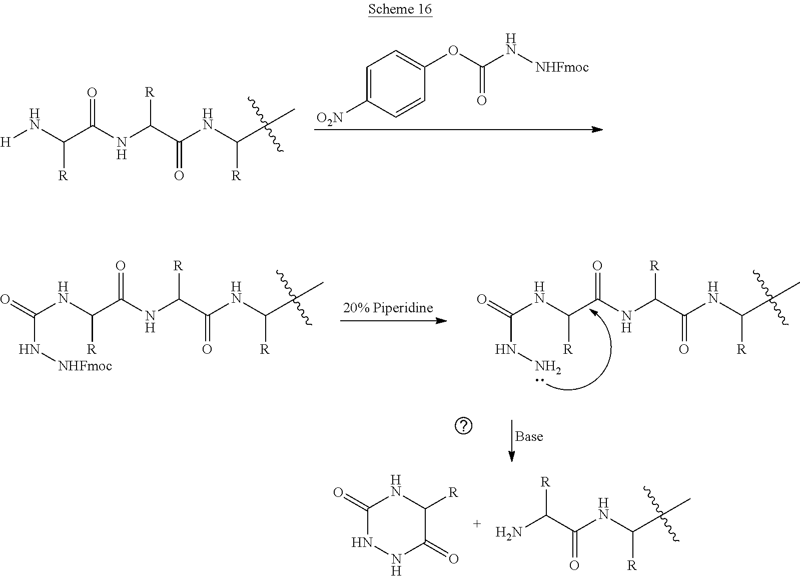

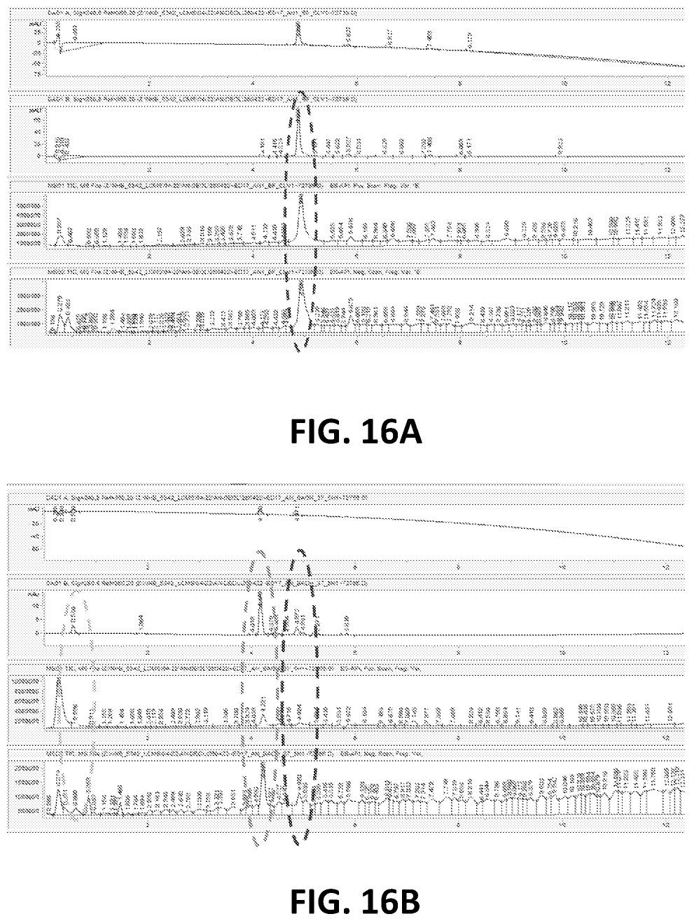

A peptide was treated with 1-((9H-fluoren-9-yl)methyl)₂-(4-nitrophenyl) hydrazine-1,2-dicarboxylate in the presence of piperidine to generate a functionalized peptide. A peptide (YGFWVY; SEQ ID NO: 1) was treated with 1% Ba(OH)₂ at 37° C. for 5 or at 60° C. for 1.5 h using the procedure described above. The degraded peptide was obtained in greater than 90% yield. FIG. 16A shows an LC-MS trace prior to treatment with Ba(OH)₂. FIG. 16B shows an LC-MS trace after treatment with Ba(OH)₂, wherein the peak circled on the right is unreacted product.

### Example 5-Base-Induced Terminal Degradation Using 1-((9H-fluoren-9-yl)methyl)₂-(2,5-dioxopyrrolidin-1-yl) hydrazine-1,2-dicarboxylate

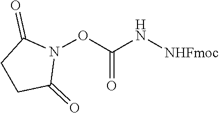

To the solution of peptide (YSEVFWVADLSFAY (SEQ ID NO: 13); 2.5 M) in dimethylformamide (DMF) (400 μL) were added 1-((9H-fluoren-9-yl)methyl)₂-(2,5-dioxopyrrolidin-1-yl) hydrazine-1,2-dicarboxylate (2.5 μM), N,N diisopropylethylamine (DIPEA) (5 μM). The mixture was stirred at room temperature for one hour. The progress of the reaction was monitored by LCMS. Upon completion of reaction, 80 μL piperidine was added and stirred further for 15 minutes and monitored by LCMS. Upon completion of reaction, the desired product was purified by reverse phase high-performance liquid chromatography (RP-HPLC) using 5-95% MeCN/H₂O, 0.1% formic acid gradient elution.

Sequencing: The aliquot of 20 μL of purified peptide-complex solution (2 mM) was dissolved in acetonitrile: water mixture (1:1) was added in 100 μL 1% Ba(OH)₂ solution and reacted at 60° C. for 2 hours. Upon completion, the reaction was quenched using aqueous acetic acid and analyzed by LCMS. The truncated peptide obtained in different yield depending upon the N-terminal amino acid.

### Example 6: Solid Phase Functionalization and Sequencing of Peptides

A peptide is functionalized on a solid support, specifically Tentagel rink amide Resin. Peptide loaded tentagel rink amide resin beads (25 μM) are added to a medium frit solid phase synthesis apparatus. The resin is suspended in 1 mL dimethylformamide (DMF) and left to swell for 10 minutes. Afterwards, 1-((9H-fluoren-9-yl)methyl) 2-(2,5-dioxopyrrolidin-1-yl) hydrazine-1,2-dicarboxylate (125 μM) and N, N diisopropylethylamine (DIPEA) (125 μM) are added. The reaction is shaken further for 2-4 hours at room temperature or overnight, as needed. The resin is washed with DMF (3×15 mL) followed by DCM (3×15 mL), and finally methanol (3×5 mL). The resin is dried. Test cleavages are treated with a TFA:TIPS:H₂O (95:2.5:2.5) cleavage cocktail for 2 hours. The resin is filtered off, and the cleaved product is concentrated. Addition of 3 mL of diethyl ether results in a white precipitate, which is purified by reverse-phase preparatory HPLC (5-95% MeCN/H₂O, 0.1% formic acid gradient elution). The coupling efficiency is checked by LC/MS.

Sequencing on solid phase: Functionalized peptide-loaded tentagel rink amide resin beads (25 μM) are suspended in 1 mL of 1% Ba(OH)₂ solution and reacted at 60° C. for 4 hours. Upon completion of reaction, the resin is washed with H₂O (3×15 mL) and methanol (3×15 mL). The resin is dried. The resin is treated with a TFA:TIPS:H₂O (95:2.5:2.5) cleavage cocktail for 2 hours. The resin is filtered off, and the cleaved product is concentrated. Addition of 1 mL of diethyl ether results in white precipitate, and the yield of the sequencing step is monitored by LC/MS.

### Example 7: Comparison of Alternative Edman Degradation Reactions

### Synthesis of bis (4-methyl-1H-pyrazol-1-yl)methanimine

Procedure A: To a glass vial equipped with a magnetic stir bar, 100 mg of cyanogen bromide (0.95 mmol) was added in and dissolved in 1-2 mL of acetone and cooled on an ice bath until later use. In a separate vial, 1.97 mmol of 4-methyl-1H-pyrazole was dissolved in 5-6 mL of ethanol, and the resulting solution was mixed in with the chilled acetone solution. The solution was stirred at 0° C. for 5 minutes, and 800 μL of 2M NaOH (aq.) was added. The vigorously stirred solution was warmed to room temperature over the course of 1 hour. A precipitate formed, and the solids were filtered and washed with cold ethanol. The resulting solids were obtained without further purification (>95% pure, 20-60% yield). Dry solvents were used in all reactions.

Procedure B: To a glass vial equipped with a magnetic stir bar, 100 mg of cyanogen bromide (0.95 mmol) was added in and dissolved in 1-2 mL of dichloromethane and stored at 4° C. until further use. In a separate vial, 1.97 mmol of 4-methyl-1H-pyrazole was dissolved in 5 mL of dichloromethane. To this, 3 mmol of triethylamine (or diisopropylethylamine) was added, and the resulting mixture was stirred for 10 minutes or until all solids dissolved. This solution was then added dropwise to the cyanogen bromide containing solution. The reaction was stirred at 25° C. for 1-18 hours. The reaction was monitored to completion by thin layer chromatography (TLC), and the reaction was condensed in vacuo and loaded onto a normal phase silica plug. The product was obtained by normal phase flash chromatography (0-60% ethyl acetate in n-heptane). The fractions containing the desired product were pooled and condensed to afford the isolated product (>95% pure, 40-85% yield). Dry solvents were used in all reactions.

### Functionalization of Peptides Using bis(4-methyl-1H-pyrazol-1-yl)methanimine

To the solution of a peptide (2 μM) in 200 μL dimethylformamide (DMF) is added bis (4-methyl-1H-pyrazol-1-yl) methanimine (2 μM), and the reaction was stirred at 50° C. for 5 h. The progress of the reaction was monitored by LCMS.

### Comparison of Peptides Modified with bis(4-methyl-1H-pyrazol-1-yl)methanimine or an N-Terminal Labeling Reagent of the Disclosure

The same peptide used for modification with bis (4-methyl-1H-pyrazol-1-yl) methanimine is treated with an N-terminal coupling reagent of the disclosure. The efficiency of labeling are compared by LC-MS using final reaction yields. The stability of modified peptides are also compared.

### EMBODIMENTS

The following non-limiting embodiments provide illustrative examples of the invention, but do not limit the scope of the invention.

Embodiment 1. A method for analyzing a biomolecule comprising: (a) providing the biomolecule comprising a detectable label coupled to an amino acid of the biomolecule; (b) detecting a signal from the detectable label coupled to the amino acid of the biomolecule; (c) coupling an N-terminal coupling reagent to an N-terminal amino acid of the biomolecule to form a modified biomolecule, wherein the modified biomolecule comprises a hydroxamic acid or a hydrazide; and (d) subjecting the modified biomolecule to conditions sufficient to remove the N-terminal amino acid from the biomolecule.

Embodiment 2. The method of embodiment 1, wherein the detectable label is a dye.

Embodiment 3. The method of embodiment 2, wherein the dye is a cyanine dye, diazo dye, organoboron dye, or a combination thereof.

Embodiment 4. The method of embodiment 2, wherein the dye is a boron-dipyrromethane (BODIPY) dye.

Embodiment 5. The method of embodiment 1, wherein the detectable label is a fluorescent label.

Embodiment 6. The method of any one of embodiments 1-5, wherein the biomolecule is a polypeptide.

Embodiment 7. The method of any one of embodiments 1-5, wherein the biomolecule is a protein.

Embodiment 8. The method of any one of embodiments 1-7, wherein the detectable label generates at least one signal or at least one signal change.

Embodiment 9. The method of embodiment 8, wherein the at least one signal or the at least one signal change is an optical signal.

Embodiment 10. The method of embodiment 8 or 9, wherein the at least one signal or the at least one signal change comprises a plurality of signals of different intensities.

Embodiment 11. The method of any one of embodiments 8-10, wherein the at least one signal or the at least one signal change comprises a plurality of signals of different frequencies or signals of different frequency ranges.

Embodiment 12. The method of any one of embodiments 8-11, wherein the at least one signal or signal change is detected with an optical detector having single-molecule sensitivity.

Embodiment 13. The method of any one of embodiments 1-12, wherein the detecting comprises fluorimetry.

Embodiment 14. The method of any one of embodiments 1-12, wherein the detecting comprises imaging.

Embodiment 15. The method of any one of embodiments 1-14, wherein the detecting identifies a sequence of the biomolecule.

Embodiment 16. The method of any one of embodiments 1-15, wherein the detectable label is coupled to an internal amino acid of the biomolecule.

Embodiment 17. The method of embodiment 16, wherein the internal amino acid to which the detectable label couples is selected from the group consisting of cysteine, lysine, tyrosine, histidine, glutamate, aspartate, tryptophan, arginine, serine, threonine, and methionine.

Embodiment 18. The method of any one of embodiments 1-17, wherein the detectable label is an amino acid specific label.

Embodiment 19. The method of embodiment 18, wherein the detectable label comprises a methionine specific label, an arginine specific label, a histidine specific label, a tyrosine specific label, a carboxylic acid R-group specific label, a lysine specific label, a cysteine specific label, a tryptophan specific label, or any combination thereof.

Embodiment 20. The method of any one of embodiments 1-19, wherein the detectable label comprises at least two types of detectable labels, each of which couples to a different type of amino acid selected from the group consisting of cysteine, lysine, tyrosine, histidine, glutamate, aspartate, tryptophan, arginine, serine, threonine, and methionine.

Embodiment 21. The method of any one of embodiments 1-19, wherein the detectable label comprises at least three types of detectable labels, each of which couples to a different type of amino acid selected from the group consisting of cysteine, lysine, tyrosine, histidine, glutamate, aspartate, tryptophan, arginine, serine, threonine, and methionine.

Embodiment 22. The method of any one of embodiments 1-19, wherein the detectable label comprises at least four types of detectable labels, each of which couples to a different type of amino acid selected from the group consisting of cysteine, lysine, tyrosine, histidine, glutamate, aspartate, tryptophan, arginine, serine, threonine, and methionine.

Embodiment 23. The method of any one of embodiments 1-19, wherein the detectable label comprises at least five types of detectable labels, each of which couples to a different type of amino acid selected from the group consisting of cysteine, lysine, tyrosine, histidine, glutamate, aspartate, tryptophan, arginine, serine, threonine, and methionine.

Embodiment 24. The method of embodiment 18, wherein the amino acid specific label comprises a non-natural amino acid specific label.

Embodiment 25. The method of embodiment 19, wherein the non-natural amino acid specific label is a phosphoserine specific label, phosphothreonine specific label, pyroglutamic acid specific label, hydroxyproline specific label, azidolysine specific label, or dehydroalanine specific label.

Embodiment 26. The method of any one of embodiments 1-25, wherein the amino acid to which the detectable label couples is a post-translationally modified amino acid.

Embodiment 27. The method of embodiment 26, wherein the post-translationally modified amino acid is citrullinated, methylated, sulfurylated, phorphorylated, succinylated, glycosylated, palmitoylated, prenylated, acylated, amidated, hydroxylated, iodinated, chlorinated, fluorinated, nitrosylated, glutathionylated, malonated, biotinylated, oxidized, reduced, or any combination thereof.

Embodiment 28. The method of any one of embodiments 1-27, wherein the modified biomolecule comprises a substituted hydroxamic acid.

Embodiment 29. The method of any one of embodiments 1-27, wherein the modified biomolecule comprises an unsubstituted hydroxamic acid.

Embodiment 30. The method of embodiment 28 or 29, wherein said removing said N-terminal amino acid from said biomolecule generates a 1,2,4-oxadiazinane-3,6-dione byproduct.

Embodiment 31. The method of embodiment 28 or 29, wherein said removing said N-terminal amino acid from said biomolecule generates a 5-substituted 1,2,4-oxadiazinane-3,6-dione byproduct.

Embodiment 32. The method of any one of embodiments 1-27, wherein the modified biomolecule comprises a hydrazide.

Embodiment 33. The method of embodiment 32, wherein said removing said N-terminal amino acid from said biomolecule generates a 1,2,4-triazine-3,6-dione byproduct.

Embodiment 34. The method of embodiment 32, wherein said removing said N-terminal amino acid from said biomolecule generates a 5-substituted 1,2,4-triazine-3,6-dione byproduct.

Embodiment 35. The method of any one of embodiments 1-34, wherein the method comprises sequencing by degradation.

Embodiment 36. The method of any one of embodiments 1-35, wherein the N-terminal coupling reagent comprises a carbamate group.

Embodiment 37. The method of any one of embodiments 1-36, wherein the conditions to remove the N-terminal amino acid from the biomolecule comprises contacting the modified biomolecule with a base.

Embodiment 38. The method of embodiment 37, wherein the base is a Ba(OH)₂.

Embodiment 39. The method of embodiment 37, wherein the base is NaOH.

Embodiment 40. The method of any one of embodiments 1-39, further comprising immobilizing the biomolecule to a support.

Embodiment 41. The method of embodiment 40, wherein the immobilizing comprises coupling a C-terminus of the biomolecule to the support.

Embodiment 42. The method of embodiment 40, wherein the immobilizing comprises coupling a cysteine thiol of the biomolecule to the support.

Embodiment 43. The method of embodiment 40, wherein the immobilizing comprises non-covalently coupling the biomolecule to a protein coupled to the support.

Embodiment 44. The method of embodiment 43, wherein the protein comprises an antibody, a T-cell receptor, a pore protein, a catalytically inactive protease, or any combination thereof.

Embodiment 45. The method of any one of embodiments 1-44, further comprising repeating (a)-(d).

Embodiment 46. The method of any one of embodiments 1-45, further comprising identifying an unlabeled amino acid of the biomolecule.

Embodiment 47. The method of any one of embodiments 1-46, wherein the at least one amino acid removed from the modified biomolecule comprises the N-terminal amino acid.

Embodiment 48. The method of any one of embodiments 1-47, wherein the N-terminal coupling agent is a compound of Formula (II), or a salt, a solvate, or a derivative thereof:

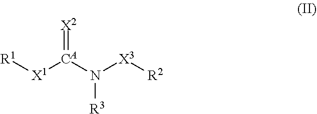

wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen, wherein the reagent modifies the N-terminal amino acid of the peptide.

Embodiment 49. The method of embodiment 48, wherein R¹ is an electron withdrawing group.

Embodiment 50. The method of embodiment 48, wherein R¹ is an electron donating group.

Embodiment 51. The method of embodiment 48, wherein R¹ is amino, alkoxy, aryl, or heteroaryl, each of which is independently unsubstituted or substituted; or hydroxy.

Embodiment 52. The method of embodiment 48, wherein R¹ is substituted phenyl.

Embodiment 53. The method of embodiment 48, wherein R¹ is nitrophenyl.

Embodiment 54. The method of any one of embodiments 48-53, wherein R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group.

Embodiment 55. The method of any one of embodiments 48-53, wherein R² comprises a silyl group.

Embodiment 56. The method of embodiment 55, wherein the silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM).

Embodiment 57. The method of any one of embodiments 48-56, wherein R² is tert-butyldimethylsilyl.

Embodiment 58. The method of any one of embodiments 48-56, wherein R² is trimethylsilyl.

Embodiment 59. The method of any one of embodiments 48-58, wherein X¹ is O.

Embodiment 60. The method of any one of embodiments 48-59, wherein X² is O.

Embodiment 61. The method of any one of embodiments 48-60, wherein X³ is O.

Embodiment 62. The method of any one of embodiments 48-61, wherein each R³ and R⁴ is independently C₁-C₉ alkyl, C₁-C₉ alkenyl, or C₁-C₉ alkynyl, each of which is independently unsubstituted or substituted.

Embodiment 63. The method of any one of embodiments 48-62, wherein R³ is hydrogen.

Embodiment 64. The method of any one of embodiments 48-63, wherein R⁴ is hydrogen.

Embodiment 65. The method of any one of embodiments 48-64, wherein the reagent has the structure:

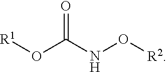

Embodiment 66. A composition comprising: (a) a peptide comprising an N-terminal amino acid; and (b) a reagent comprising a structure of Formula (II), or a salt, a solvate, or a derivative thereof:

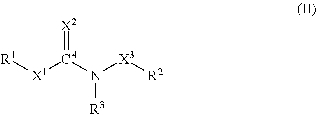

wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen, wherein the reagent modifies the N-terminal amino acid of the peptide.

Embodiment 67. The composition of embodiment 66, wherein R¹ is an electron withdrawing group.

Embodiment 68. The composition of embodiment 66, wherein R¹ is an electron donating group.

Embodiment 69. The composition of embodiment 66, wherein R¹ is amino, alkoxy, aryl, or heteroaryl, each of which is independently unsubstituted or substituted; or hydroxy.

Embodiment 70. The composition of embodiment 66, wherein R¹ is substituted phenyl.

Embodiment 71. The composition of embodiment 70, wherein R¹ is nitrophenyl.

Embodiment 72. The composition of any one of embodiments 66-71, wherein R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group.

Embodiment 73. The composition of any one of embodiments 66-71, wherein R² comprises a silyl group.

Embodiment 74. The composition of embodiment 73, wherein the silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM).

Embodiment 75. The composition of any one of embodiments 66-74, wherein R² is tert-butyldimethylsilyl.

Embodiment 76. The composition of any one of embodiments 66-74, wherein R² is trimethylsilyl.

Embodiment 77. The composition of any one of embodiments 66-76, wherein X¹ is O.

Embodiment 78. The composition of any one of embodiments 66-77, wherein X² is O.

Embodiment 79. The composition of any one of embodiments 66-78, wherein X³ is O.

Embodiment 80. The composition of any one of embodiments 66-79, wherein each R³ and R⁴ is independently C₁-C₉ alkyl, C₁-C₉ alkenyl, or C₁-C₉ alkynyl, each of which is independently unsubstituted or substituted.

Embodiment 81. The composition of any one of embodiments 66-80, wherein R³ is hydrogen.

Embodiment 82. The composition of any one of embodiments 66-81, wherein R⁴ is hydrogen.

Embodiment 83. The composition of any one of embodiments 66-82, wherein the reagent has the structure:

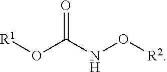

Embodiment 84. The composition of any one of embodiments 66-83, further comprising an organic solvent.

Embodiment 85. The composition of embodiment 84, wherein the organic solvent is dimethylsulfoxide (DMSO).

Embodiment 86. The composition of embodiment 84, wherein the organic solvent is dimethylformamide (DMF).

Embodiment 87. The composition of any one of embodiments 66-86, wherein R² is configured for cleavage by a base.

Embodiment 88. The composition of embodiment 87, wherein the base is a halide.

Embodiment 89. The composition of embodiment 88, wherein the halide is fluoride.

Embodiment 90. The composition of any one of embodiments 66-89, wherein the reagent is configured to cleave the N-terminal amino acid from the peptide.

Embodiment 91. A method comprising: (a) providing a polypeptide immobilized to a support, wherein the polypeptide comprises at least one labeled internal amino acid; (b) detecting at least one signal or signal change from the polypeptide immobilized to the support to identify at least a portion of a sequence of the polypeptide; and (c) subjecting the polypeptide to conditions sufficient to remove at least one amino acid from the polypeptide to form a cleaved polypeptide via a hydroxamic acid or a hydrazide intermediate.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for analyzing a biomolecule comprising:
(A) providing said biomolecule comprising a detectable label coupled to an amino acid of said biomolecule;
(B) detecting a signal from said detectable label coupled to said amino acid of said biomolecule;
(C) coupling an N-terminal coupling reagent to an N-terminal amino acid of said biomolecule to form a modified biomolecule, wherein said modified biomolecule comprises an hydroxamic acid or a hydrazide; and
(D) subjecting said modified biomolecule to conditions sufficient for removing said N-terminal amino acid from said biomolecule.

2. The method of claim 1, wherein said detectable label is a dye.

3. The method of claim 2, wherein the dye is a cyanine dye, diazo dye, organoboron dye, or a combination thereof.

4. The method of claim 2, wherein the dye is a boron-dipyrromethane (BODIPY) dye.

5. The method of claim 1, wherein said detectable label is a fluorescent label.

6. The method of claim 1, wherein said biomolecule is a polypeptide.

7. The method of claim 1, wherein said biomolecule is a protein.

8. The method of claim 1, wherein said detectable label generates at least one signal or at least one signal change.

9. The method of claim 8, wherein said at least one signal or said at least one signal change is an optical signal.

10. The method of claim 8, wherein said at least one signal or said at least one signal change comprises a plurality of signals of different intensities.

11. The method of claim 8, wherein said at least one signal or said at least one signal change comprises a plurality of signals of different frequencies or signals of different frequency ranges.

12. The method of claim 8, wherein said at least one signal or signal change is detected with an optical detector having single-molecule sensitivity.

13. The method of claim 1, wherein said detecting comprises fluorimetry.

14. The method of claim 1, wherein said detecting comprises imaging.

15. The method of claim 1, wherein said detecting identifies a sequence of said biomolecule.

16. The method of claim 1, wherein said detectable label is coupled to an internal amino acid of said biomolecule.

17. The method of claim 16, wherein said internal amino acid to which said detectable label couples is selected from the group consisting of cysteine, lysine, tyrosine, histidine, glutamate, aspartate, tryptophan, arginine, serine, threonine, and methionine.

18. The method of claim 1, wherein said detectable label is an amino acid specific label.

19. The method of claim 18, wherein said detectable label comprises a methionine specific label, an arginine specific label, a histidine specific label, a tyrosine specific label, a carboxylic acid R-group specific label, a lysine specific label, a cysteine specific label, a tryptophan specific label, or any combination thereof.

20. The method of claim 1, wherein said detectable label comprises at least two types of detectable labels, each of which couples to a different type of amino acid selected from the group consisting of cysteine, lysine, tyrosine, histidine, glutamate, aspartate, tryptophan, arginine, serine, threonine, and methionine.

21. The method of claim 1, wherein said detectable label comprises at least three types of detectable labels, each of which couples to a different type of amino acid selected from the group consisting of cysteine, lysine, tyrosine, histidine, glutamate, aspartate, tryptophan, arginine, serine, threonine, and methionine.

22. The method of claim 1, wherein said detectable label comprises at least four types of detectable labels, each of which couples to a different type of amino acid selected from the group consisting of cysteine, lysine, tyrosine, histidine, glutamate, aspartate, tryptophan, arginine, serine, threonine, and methionine.

23. The method of claim 1, wherein said detectable label comprises at least five types of detectable labels, each of which couples to a different type of amino acid selected from the group consisting of cysteine, lysine, tyrosine, histidine, glutamate, aspartate, tryptophan, arginine, serine, threonine, and methionine.

24. The method of claim 18, wherein said amino acid specific label comprises a non-natural amino acid specific label.

25. The method of claim 19, wherein said non-natural amino acid specific label is a phosphoserine specific label, phosphothreonine specific label, pyroglutamic acid specific label, hydroxyproline specific label, azidolysine specific label, or dehydroalanine specific label.

26. The method of claim 1, wherein said amino acid to which said detectable label couples is a post-translationally modified amino acid.

27. The method of claim 26, wherein said post-translationally modified amino acid is citrullinated, methylated, sulfurylated, phorphorylated, succinylated, glycosylated, palmitoylated, prenylated, acylated, amidated, hydroxylated, iodinated, chlorinated, fluorinated, nitrosylated, glutathionylated, malonated, biotinylated, oxidized, reduced, or any combination thereof.

28. The method of claim 1, wherein the modified biomolecule comprises a substituted hydroxamic acid.

29. The method of claim 1, wherein the modified biomolecule comprises an unsubstituted hydroxamic acid.

30. The method of claim 28, wherein said removing said N-terminal amino acid from said biomolecule generates a 1,2,4-oxadiazinane-3,6-dione byproduct.

31. The method of claim 28, wherein said removing said N-terminal amino acid from said biomolecule generates a 5-substituted 1,2,4-oxadiazinane-3,6-dione byproduct.

32. The method of claim 1, wherein the modified biomolecule comprises a hydrazide.

33. The method of claim 32, wherein said removing said N-terminal amino acid from said biomolecule generates a 1,2,4-triazine-3,6-dione byproduct.

34. The method of claim 32, wherein said removing said N-terminal amino acid from said biomolecule generates a 5-substituted 1,2,4-triazine-3,6-dione byproduct.

35. The method of claim 1, wherein the method comprises sequencing by degradation.

36. The method of claim 1, wherein said N-terminal coupling reagent comprises a carbamate group.

37. The method of claim 1, wherein said conditions to remove said N-terminal amino acid from said biomolecule comprises contacting said modified biomolecule with a base.

38. The method of claim 37, wherein said base is a Ba(OH)₂.

39. The method of claim 37, wherein said base is NaOH.

40. The method of claim 1, further comprising immobilizing said biomolecule to a support.

41. The method of claim 40, wherein said immobilizing comprises coupling a C-terminus of said biomolecule to said support.

42. The method of claim 40, wherein said immobilizing comprises coupling a cysteine thiol of said biomolecule to said support.

43. The method of claim 40, wherein said immobilizing comprises non-covalently coupling said biomolecule to a protein coupled to said support.

44. The method of claim 43, wherein said protein comprises an antibody, a T-cell receptor, a pore protein, a catalytically inactive protease, or any combination thereof.

45. The method of claim 1, further comprising repeating (A)-(D).

46. The method of claim 1, further comprising identifying an unlabeled amino acid of said biomolecule.

47. The method of claim 1, wherein said at least one amino acid removed from said modified biomolecule comprises said N-terminal amino acid.

48. The method of claim 1, wherein the N-terminal coupling agent is a compound of Formula (II), or a salt, a solvate, or a derivative thereof: 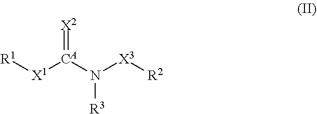
wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen,
wherein said reagent modifies said N-terminal amino acid of said peptide.

49. The method of claim 48, wherein R¹ is an electron withdrawing group.

50. The method of claim 48, wherein R¹ is an electron donating group.

51. The method of claim 48, wherein R¹ is amino, alkoxy, aryl, or heteroaryl, each of which is independently unsubstituted or substituted; or hydroxy.

52. The method of claim 48, wherein R¹ is substituted phenyl.

53. The method of claim 48, wherein R¹ is nitrophenyl.

54. The method of claim 48, wherein R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group.

55. The method of claim 48, wherein R² comprises a silyl group.

56. The method of claim 55, wherein said silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM).

57. The method of claim 48, wherein R² is tert-butyldimethylsilyl.

58. The method of claim 48, wherein R² is trimethylsilyl.

59. The method of claim 48, wherein X¹ is O.

60. The method of claim 48, wherein X² is O.

61. The method of claim 48, wherein X³ is O.

62. The method of claim 48, wherein each R³ and R⁴ is independently C₁-C₉ alkyl, C₁-C₉ alkenyl, or C₁-C₉ alkynyl, each of which is independently unsubstituted or substituted.

63. The method of claim 48, wherein R³ is hydrogen.

64. The method of claim 48, wherein R⁴ is hydrogen.

65. The method of claim 48, wherein said reagent has the structure:

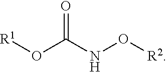

66. A composition comprising:
(A) a peptide comprising an N-terminal amino acid; and
(B) a reagent comprising a structure of Formula (II), or a salt, a solvate, or a derivative thereof: 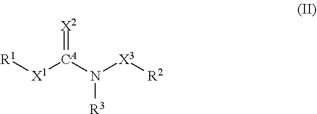
wherein:
R¹ is an electron donating group or an electron withdrawing group;
R² is a leaving group;
X¹ is O, S, SO, SR⁴, Se, SeO, SeR⁴, or NR⁴;
X² is O, S, SR⁴, SOR⁴, SO₂R⁴, Se, SeR⁴, SeOR⁴, SeO₂R⁴ or NR⁴; and
X³ is O, S, Se, or NR⁴;
each instance of R³ and R⁴ is independently selected from the group consisting of alkyl, alkenyl, or alkynyl, each of which is independently unsubstituted or substituted; or hydrogen,
wherein said reagent modifies said N-terminal amino acid of said peptide.

67. The composition of claim 66, wherein R¹ is an electron withdrawing group.

68. The composition of claim 66, wherein R¹ is an electron donating group.

69. The composition of claim 66, wherein R¹ is amino, alkoxy, aryl, or heteroaryl, each of which is independently unsubstituted or substituted; or hydroxy.

70. The composition of claim 66, wherein R¹ is substituted phenyl.

71. The composition of claim 70, wherein R¹ is nitrophenyl.

72. The composition of claim 66, wherein R² comprises an acetyl group, a benzoyl group, a benzyl group, a tosyl group, a triphenylmethane group, a methylthiomethyl ether group, a carbobenzyloxy group, a p-methoxybenzyl ether (PMB) group, a 9-fluorenylmethyloxycarbonyl (FMOC) group, a pivaloyl group, a tetrahydropyranyl (THP) group, a silyl group, a methyl ether, an ethoxy ethyl, or a sulfonamide group.

73. The composition of claim 66, wherein R² comprises a silyl group.

74. The composition of claim 73, wherein said silyl group comprises trimethylsilyl (TMS), triethylsilyl (TES), tert-butyldimethylsilyl (TBDMS), tert-Butyldiphenylsilyl (TBDPS), triisopropylsilyl (TIPS), or triisopropylsilyloxymethyl (TOM).

75. The composition of claim 66, wherein R² is tert-butyldimethylsilyl.

76. The composition of claim 66, wherein R² is trimethylsilyl.

77. The composition of claim 66, wherein X¹ is O.

78. The composition of claim 66, wherein X² is O.

79. The composition of claim 66, wherein X³ is O.

80. The composition of claim 66, wherein each R³ and R⁴ is independently C₁-C₉ alkyl, C₁-C₉ alkenyl, or C₁-C₉ alkynyl, each of which is independently unsubstituted or substituted.

81. The composition of claim 66, wherein R³ is hydrogen.

82. The composition of claim 66, wherein R⁴ is hydrogen.

83. The composition of claim 66, wherein said reagent has the structure:

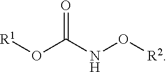

84. The composition of claim 66, further comprising an organic solvent.

85. The composition of claim 84, wherein said organic solvent is dimethylsulfoxide (DMSO).

86. The composition of claim 84, wherein said organic solvent is dimethylformamide (DMF).

87. The composition of claim 66, wherein R² is configured for cleavage by a base.

88. The composition of claim 87, wherein said base is a halide.

89. The composition of claim 88, wherein said halide is fluoride.

90. The composition of claim 66, wherein said reagent is configured to cleave said N-terminal amino acid from said peptide.

91. A method comprising:
(A) providing a polypeptide immobilized to a support, wherein said polypeptide comprises at least one labeled internal amino acid;
(B) detecting at least one signal or signal change from said polypeptide immobilized to said support to identify at least a portion of a sequence of said polypeptide; and
(C) subjecting said polypeptide to conditions sufficient to remove at least one amino acid from said polypeptide to form a cleaved polypeptide via a hydroxamic acid or a hydrazide intermediate.

92. The method of claim 29, wherein said removing said N-terminal amino acid from said biomolecule generates a 1,2,4-oxadiazinane-3,6-dione byproduct.

93. The method of claim 29, wherein said removing said N-terminal amino acid from said biomolecule generates a 5-substituted 1,2,4-oxadiazinane-3,6-dione byproduct.


SEQUENCE LISTING (13 sequences)

SEQ ID NO: 1 - protein, 6 aa, synthetic construct
YGFWVY

SEQ ID NO: 2 - protein, 6 aa, synthetic construct
WYFWYV

SEQ ID NO: 3 - protein, 6 aa, synthetic construct
QYFWVY

SEQ ID NO: 4 - protein, 6 aa, synthetic construct
EYFWVY

SEQ ID NO: 5 - protein, 6 aa, synthetic construct
DYFWVY

SEQ ID NO: 6 - protein, 6 aa, synthetic construct
HYFWVY

SEQ ID NO: 7 - protein, 6 aa, synthetic construct
CYFWVY

SEQ ID NO: 8 - protein, 6 aa, synthetic construct
SYFWVY

SEQ ID NO: 9 - protein, 4 aa, synthetic construct
KYFW

SEQ ID NO: 10 - protein, 7 aa, synthetic construct
PFFAYVY

SEQ ID NO: 11 - protein, 14 aa, synthetic construct
KDDYAGGGAAGKDA

SEQ ID NO: 12 - protein, 7 aa, synthetic construct
YFAVALV

SEQ ID NO: 13 - protein, 14 aa, synthetic construct
YSEVFWVADLSFAY